(19) United States
(12) Patent Application Publication
(10) Pub. No.: US 2023/0398153 A1
(43) Pub. Date: Dec. 14, 2023

(54) A METHOD FOR EFFICIENT MICROGLIA REPLACEMENT

(71) Applicant: The Board of Trustees of the Leland Stanford Junior University, Stanford, CA (US)

(72) Inventors: Marius Wernig, Palo Alto, CA (US); Yohei Shibuya, Redwood City, CA (US)

(21) Appl. No.: 18/026,789

(22) PCT Filed: Sep. 24, 2021
(86) PCT No.: PCT/US2021/052060

§ 371 Date: Mar. 16, 2023

Related U.S. Application Data
(60) Provisional application No. 63/083,657, filed on Sep. 25, 2020.

(51) Int. Cl. A61K 35/28; A61K 31/255; A61K 45/06; A61P 27/04

(52) U.S. Cl. CPC A61K 35/28; A61K 31/255; A61K 45/06; A61P 27/04

(57) ABSTRACT

Methods are provided for the efficient replacement of endogenous microglia with circulation-derived cells derived from the bone marrow in an individual, the method comprising hematopoietic stem cell transplantation, and upregulation of CDMC repopulation, wherein (i) endogenous hematopoietic stem cells are ablated, (ii) exogenous hematopoietic stem cells are introduced to the patient; and (iii) a glial cell conditioning agent is administered to enhance replacement of endogenous microglia with circulation-derived cells.

### CROSS REFERENCE TO RELATED APPLICATION

The present application is a 371 and claims the benefit of PCT Application No. PCT/US2021/052060, filed Sep. 24, 2021, which claims the benefit of U.S. Provisional Patent Application No. 63/083,657 filed Sep. 25, 2020, which applications are incorporated herein by reference in their entirety.

### INCORPORATION BY REFERENCE OF SEQUENCE LISTING

A Sequence Listing is provided herewith as a Sequence Listing text, S20-137_STAN-1752_SEQLIST_ST25, created on Mar. 16, 2023, and having a size of 45,274 bytes. The contents of the Sequence Listing text are incorporated herein by reference in their entirety.

### BACKGROUND

Microglia are the tissue resident macrophages of the central nervous system (CNS) and are important for normal CNS development and function by processes such as synaptic pruning and regulating neurogenesis. Recently, microglia have been further implicated in both neurodevelopmental and neurodegenerative diseases primarily driven by the discovery that mutations in microglial genes can cause severe brain pathology. Moreover, recent studies have identified disease-associated microglia (DAM), also termed cells with microglial neurodegenerative (MGnD) phenotype, that are a subset of microglia distinct from homeostatic microglia with a common transcriptional profile in various neurodegenerative and aging brains. Those studies highlight the importance of microglia in the initiation and progression of CNS disease.

Microglia dynamically survey the brain parenchyma utilizing their fine, elaborate processes with each individual cell assigned a specific brain volume leading to an evenly tiled distribution of these cells throughout the CNS. The mechanisms responsible for the remarkably robust microglial spacing are not well understood but the NF-κB pathway has recently been shown to be involved in establishing spacing following regeneration. Microglia originate from yolk sac hematopoiesis and populate the brain during early development. In the adult brain, microglia are long-lived and are not exchanged from circulating blood cells under physiological conditions. Upon experimental depletion, microglia are regenerated from cells within the brain most likely from the few remaining microglia. However, under certain experimental conditions, adult bone marrow-derived cells are able to enter the neonatal or adult brain. For instance, perinatal bone marrow transplantation (HSCT) of wild type (WT) cells into genetic knock-out mice lacking genes essential for microglia like PU.1 or Csf1r rescues lethality and yields high microglial chimerism. Another genetic model enabling an intracerebral drug injection-induced acute microglial cell death allows similarly efficient incorporation of WT circulation-derived myeloid cells (CDMCs) into the CNS. Both irradiation and chemotherapy-mediated HSCT in non-genetic mouse models leads to CDMC engraftment but reported degrees of chimerism vary greatly.

Several reports have established that CDMCs engrafted into the brain have a very similar but distinct gene expression profile from endogenous microglia. A recent comprehensive study elegantly demonstrated that various hematopoietic cells can engraft into the neonatal brain of microglia-deficient mice and transplanted cells closely resemble microglia; however the engrafted cells retain a signature of their origin, with yolk sac cells being closer to microglia than cells from definitive hematopoiesis.

### SUMMARY OF THE INVENTION

Methods are provided for the efficient replacement of endogenous microglia with circulation-derived cells from a bone marrow transplantation in an individual, the replacement method comprising hematopoietic stem cell transplantation (HSCT), and upregulation of CDMC repopulation, wherein (i) endogenous stem cells are ablated, (ii) exogenous stem cells are introduced to the patient; and following (ii), step (iii) a microglial cell conditioning agent is administered.

Microglial cell conditioning agents of interest include inhibitors of colony stimulating factor 1 (CSF1), signaling. In some embodiments the conditioning agent is a brain penetrant agent, e.g. small molecule drugs. In some embodiment the microglial cell conditioning agent is an inhibitor of human CSF1R.

The interval of time between infusion of HSC and administration of the microglial cell conditioning agent may be at least about 3 days, at least about 7 days, at least about 10 days, at least about 14 days, at least about 16 days, at least about 21 days, at least about 25 days, at least 28 days. The interval may be not more than about 48 days. In some embodiments the interval of time is from about 14 to about 21 days, or from about 14 to about 28 days.

The conditioning agent is administered daily, twice daily, every other day, every third day, etc. for a period of time sufficient to effect the desired reduction in numbers of brain resident microglial cells, for at least 1 day, 2 days, 3, 4, 5, 6, 7, 8 or more days. In some embodiments from 4-10 days is sufficient.

Following microglial replacement, the methods of the invention provide for high level chimerism, e.g. at least about 25% donor-derived brain-resident CDMC following the transplantation procedure, at least about 35%, at least about 50%, at least about 65%, at least about 75%, at least about 85%, at least about 90%, or more. The presence of donor-derived cells in the brain increases following the second conditioning step, and reaches a high level of chimerism after about 4, about 5, about 6, about 7, about 8 weeks. The level of chimerism can stay high for at least about 4 weeks, at least about 8 weeks, at least about 12 week, at least about 16 week, or longer.

In some embodiments the infused cells are whole bone marrow cells, or purified fractions thereof. In some embodiments the infused cells are purified HSC. HSC can be isolated from a donor, including the individual selected for treatment, or can be generated from, for example, pluripotent stem cells such as iPSC. Donor HSC may be genetically altered in order to introduce genes useful in the differentiated cell, e.g. expression of therapeutic proteins, repair of a genetic defect in an individual, selectable marker, etc., or genes useful in selection against undifferentiated ES cells. Cells may also be genetically modified to enhance survival, control proliferation, and the like. Genes introduced for expression are optionally linked to a promoter that is selectively active in microglial cells, including without limitation TMEM119, Sall1, Cx3Cr1, etc.

In some embodiments the individual treated with the methods disclosed herein suffers from a neurologic condition, including without limitation Alzheimer's disease; a brain cancer; a lysosomal storage disease; etc. In some embodiments the neurologic condition is a lysosomal storage disease, including without limitation Gaucher disease. In some embodiments the HSC are genetically modified to correct the enzymatic defect of the lysosomal storage disease, e.g. an inborn error of metabolism causing a neurologic condition. A specific example is the transplantation of cells that express functional galacto-cerebrosidase (GCase), encoded by the GBA1 gene for transplantation of HSC into an individual with Gaucher disease; either from a normal endogenous gene or from genetic correction of a defective gene. In some embodiments the individual is treated at around the time of disease onset. In some embodiments the individual is diagnosed genetically and treated prior to disease onset. The demonstration shown herein that microglia replacement can rectify CNS pathology is highly relevant for neural regeneration and therapy for neurological diseases.

### BRIEF DESCRIPTION OF THE FIGURES

FIG. 1. Microglia replacement after bone marrow transplantation is a slow, inefficient and variable process. (A) Schematic overview of bone marrow transplantation (HSCT). Recipient mice were received intraperitoneal (IP) injection of busulfan for 5 days at 25 mg/kg/day (i.e. 125 mg/kg total dose) and then transplanted with GFP-expressing bone marrow cells via intravenous (IV) injection. Donor chimerism in peripheral blood (PB) and brain myeloid cells was analyzed at the indicated time points. (B and C) Donor chimerism in PB leukocytes (CD45+ cells) and brain myeloid cells (CD45+CD11b+ cells) was analyzed by flow cytometry at the indicated time point. Results showed slow and variable incorporation of GFP+ cells in the brain. GFP chimerism in PB leukocytes (B) (Mean±SEM, n=5-20/time point, ***p<0.001, ns: not significant, ANOVA). GFP chimerism in brain myeloid cells (C) (Mean±SEM, n=5/time point, *p<0.05, **p<0.01, ns: not significant, ANOVA). (D) Flow cytometry analysis of GFP+bone marrow cells 14 days after direct intracerebral (IC) injection with (bottom) and without (top) PLX5622 pre-treatment of recipient mice. Increased survival of myeloid cells in microglia-depleted brains. Representative flow cytometry plots are shown. (E) Representative images of GFP+ and Iba-1+ cells and DAPI in the brain 14 days post IC injection. Microglial differentiation of BM cells requires absence of endogenous microglia. The right panels are enlarged images of the boxed areas in the left panels. Scale bars: 500 μm (left), 50 μm (right). (F) Quantification of transplanted GFP+bone marrow cells in coronal brain sections. Left: Percentage of ramified GFP+ cells. Right: Maximum migration distance of GFP+ cells from the injection site. Horizontal bars represent mean values (n=3/group, 3 brain slices/animal were quantified. **p<0.01, ***p<0.001, Student's t-test). (G) Schematic overview of HSCT with or without busulfan and/or PLX5622 pre-treatment. GFP-expressing bone marrow cells were transplanted by IV injection. (H) Microglia chimerism was analyzed by flow cytometry 14 days post IV injection. Representative flow cytometry plots are shown. PLX5622 pre-treatment did not result in substantial improvement of myeloid brain incorporation.

FIG. 2. A transient treatment with PLX5622 after HSCT results in efficient and stable donor myeloid chimerism in the brain. (A) Schematic overview of HSCT with PLX5622 treatment. Recipient mice were treated with PLX5622 or vehicle control (CTRL) for 10 days starting on D28. Donor chimerism was analyzed at the indicated time points. (B) Flow cytometry gating for analysis of myeloid chimerism in the brain. (C) Myeloid chimerism in the brain was analyzed by flow cytometry at the indicated time points. (Horizontal bars represent median values, n=5-6/time point, **p<0.01, ***p<0.001, Student's t-test). (D) Representative images of GFP+ and Iba-1+ cells in the cortex on D83. The vast majority of Iba-1+ cells were GFP+ in the PLX5622-treated group. Scale bars: 50 μm. (E) Compilation of a full sagittal section showing stained for GFP and DAPI on D83 illustrates homogeneous and brain-wide incorporation of CDMCs in the PLX5622-treated group. Scale bars: 1 mm. (F) Quantification of GFP+ and Iba-1+ cells in the indicated brain regions on D83. Ob: olfactory bulb, Cx: cortex, Hp: hippocampus, BS: brain stem (Mean±SEM, n=6/group, 3 brain sections/animal were quantified. ***p<0.001, Student's t-test Student's t-test). (G) Schematic overview of experiment to test earlier (D14) PLX5622 treatment. (H) PLX5622 pre-treatment starting on D14 is sufficient to generate high chimerism of CDMCs in the brain as determined by flow cytometry on D34. (Horizontal bars represent mean values. n=4/group, ***p<0.001, Student's t-test). (I) Representative images of GFP+ and TMEM119+ cells in the cortex on d83. Scale bars: 50 μm.

FIG. 3. CDMCs are morphologically and functionally similar but distinct from microglia. (A) Overlaid density plots of CDMCs (red) of HSCT+PLX5622 protocol (HSCT+P) at the indicated time points and myeloid cells of untransplanted mice (gray). (B) Representative images of Iba-1+ cells in the cortex of untransplanted mice (−), mice after HSCT+P (12 weeks after transplantation), and mice after HSCT+P (24 weeks after transplantation). Lower panels are enlarged images of the boxed areas in the upper panels. Scale bars: 50 μm (upper), 25 m (lower). (C) Quantitative, morphological analysis of Iba-1+ in the indicated samples. Left: total length of processes. Center: number of branch points. Right: cell density (Horizontal bars represent median values, n=3/group, 3 brain slices/animal were quantified, ***p<0.001, ns: not significant. ANOVA). (D) Immunofluorescence staining for Iba-1 and the activated microglia marker CD68 in untransplanted (−) and HSCT+P treated mice on D83. Representative images are shown. Scale bars: 50 μm. (E) Quantification of CD68+ cells in the indicated mice shows stronger staining in CDMCs than microglia. Left: fluorescence intensity of CD68. Right: percentage of CD68+ cells in Iba-1+ cells (Horizontal bars represent median values, n=3/group, 3 brain slices/animal, ***p<0.001, Student's t-test). (F) Schematic overview of in vitro phagocytosis assay of brain myeloid cells. Phagocytosis of pH-sensitive fluorescent beads was analyzed by flow cytometry. (G) Mean fluorescence intensity (MFI) of pH-sensitive fluorescent beads in the indicated samples 1 and 4 hours of incubation (Mean±SEM, n=8, *p<0.05, ***p<0.001, ns: not significant, ANOVA)

FIG. 4. Transcriptional signature of CDMCs after HSCT+P is closer to microglia than CDMCs after conventional HSCT. (A) PCA plot for primary microglia (n=4, green) and CDMCs after HSCT+P 12 weeks after transplantation (n=6, red) using 2,500 most variable genes. Ellipses demarcate 95% confidence interval for assigned clusters. (B) Volcano plot showing differentially expressed genes (DEGs) between microglia and CDMCs. DEGs (P s 0.05, Wald test; fold changes 2) up- or down-regulated in CDMCs are indicated by red or blue. (C) Heatmap of top 100 DEGs between microglia and CDMCs. (D) Expression of select, known microglia-specific markers (Butovsky et al., 2014) in microglia (n=4) and CDMCs (n=6). Data are Mean±SEM, *p<0.05, **p<0.01, ns: not significant, Student's t-test). (E) PCA plot comparing our dataset (empty circles) and a published dataset (filled circles) of various microglia-like cell populations (Bennett et al., 2018). Untransplanted cultured microglia (Cultured MG, n=3, light green), untransplanted intact microglia (WT MG, n=7, dark brown), Intracranial transplanted (ICT)-P5 microglia (n=4, light brown), ICT-cultured microglia (ICT cultured MG, n=4, dark green), ICT-adult microglia (ICT-adult MG, n=2, dark red), ICT-yolk sac cells (ICT-YS, n=3, orange), ICT-fetal brain cells FB (ICT-FB, n=5, pink), ICT-fetal liver cells (ICT-FL, n=7, red), ICT-BM cells (ICT-BM, n=3, green), ICT-peripheral blood (ICT-Blood, n=3, light green), microglia in this study (Microglia, n=4, light green with dot lines) and CDMCs in this study (CDMCs, n=6, light red with dot lines). For data plot, 2,500 most variable genes were used. Approximately unbiased P value using PVclust package; bootstrap, n=10,000.

FIG. 5. Efficiently engrafted CDMCs are primarily derived from expanded residual cells rather than from continued influx. (A) Surface expression of CSF1R was lower in CDMCs than in microglia based on flow cytometry analysis on D28 (Mean±SEM n=4/group, *p<0.05, Student's t-test). (B) Time-course analysis of CD45+CD11b+GFP− (endogenous microglia) and CD45+CD11b+GFP+ cells (CDMCs) after HSCT during and after PLX5622 treatment. Number of the indicated cell populations was analyzed by flow cytometry (n=3-4/time point). (C) Ki67+GFP+ cells in the brain were quantified using immunofluorescence microscopy during and after PLX5622 treatment. (D) Representative images of Iba-1+, GFP+, Ki67+ cells and DAPI on D41. Red arrow heads indicate Ki67+ cells. Scale bars: 10 um. (F) Schematic of the HSCT+P experimental protocol (E) Strategy to label the PB in a different color than brain-engrafted CDMCs. Cx3cr1::CreER; Rosa26::loxStoplox::tdTomato; Ubc::EGFP mice served as donors for HSCT into WT mice. Upon injection of tamoxifen (TAM), donor-derived CX3CR1+ cells expressed tdTomato. TAM was IP-injected on D48. Expression levels of tdTomato in myeloid cells of PB and the brain were analyzed on the indicated time points. (G) Due to cell turn over, peripheral CX3CR1+ blood cells were expected to lose tdTomato expression over time. Should the CDMCs be primarily derived from pre-existing brain resident CDMCs, they were expected to remain tdTomato+ over time. (H) Expression of tdTomato in GFP+ myeloid cells in PB and the brain (CDMCs) were analyzed by flow cytometry at the indicated time point. PB cells lost tdTomato expression by 35 days after TAM injection whereas brain CDMCs were stably tdTomato+ (Horizontal bars represent mean values. n=3/time point, ***p<0.001, ns: not significant, ANOVA). (I) Representative images of GFP+ and tdTomato+ cells in the cortex on D132, i.e. 84 days after tamoxifen injection. Scale bar: 50 m. (J) Schematic of HSCT with PLX5622 to study repopulation of CDMCs after acute depletion. (K) Expected fluorophore behavior among the different cell populations. (L) Expression of GFP and tdTomato in brain myeloid cells was analyzed by flow cytometry at the indicated time points. (n=3/time point).

FIG. 6. Hematopoietic stem cells have the highest capacity to become brain CDMCs after BMT (A) Schematic representation of the 8 different hematopoietic subpopulations isolated for this experiment along with key surface markers used for sorting. HSC: hematopoietic stem cell, CMP: common myeloid progenitor, GMP: granulocyte/macrophage progenitor, MEP: Megakaryocyte/erythrocyte progenitor, MDP/CDP: Macrophage-Dendritic Progenitor/Common Dendritic Progenitor. (B) Schematic of competitive transplantation using GFP-labeled hematopoietic populations (12.5 k cells) and non-labeled whole bone marrow (WBM) (200 k cells). (C) Percent GFP+ cells among the brain's CD45+CD11b+ cell population was analyzed by flow cytometry on D28. SP: spleen (Horizontal bars represent median values, n=4/population. *p<0.05, **p<0.01. p-values were calculated using a one sample t-test vs hypothetical value of 0). (D) Schematic of competitive transplantation using GFP-labeled hematopoietic populations (12.5 k cells) and Kusabira orange (KUO)-labeled competitor cells (200 k cells). (E) Percent GFP+ cells and KUO+ cells among the brain's CD45+CD11b+ cell population was analyzed by flow cytometry on D48 (Horizontal bars represent median values, n=4/population). (F) Overlaid density plots of WBM-CDMCs (red), HSC-CDMCs (blue) and microglia (gray) at the indicated time points. (G) Representative pictures of GFP+ and Iba-1+ cells in the cortex of WBM-transplanted mice (left), and HSC-transplanted mice (right). Scale bars: 50 μm. (H) Quantitative, morphological analysis of Iba-1+ in the indicated samples. Left: total length of processes. Right: number of branch points (Horizontal bars represent median values, n=3/group, 3 brain slices/animal were quantified, ***p<0.001, ns: not significant. ANOVA). (I) Immunofluorescence staining for Iba-1 and the activated microglia marker CD68 in untransplanted mice (microglia) and HSC-transplanted (HSC-CDMC) mice on D48. Representative images are shown. Scale bars: 50 μm. (J) Quantification of CD68+ cells in the indicated mice. Left: fluorescence intensity of CD68. Right: percentage of CD68+ cells in Iba-1+ cells (Horizontal bars represent median values, n=3/group, 3 brain slices/animal, ***p<0.001, Student's t-test). (K) Unsupervised hierarchical clustering of microglia, WBM-CDMCs, and HSC-CDMCs.

FIG. 7. The aged brain environment induces aging-related transcriptome and morphology in young CDMCs. (A) Schematic of experimental HSCT outline. (B) PCA plot for endogenous microglia and CDMCs in the brain of young and aged mice. (C and D) Correlation of changes in gene expression between aged microglia and young microglia (y-axis) and between CDMCs in aged- and young brain (x-axis). DEGs in CDMCs are shown. CDMCs were derived from young bone marrow cells (C) or aged bone marrow cells (D). (E) Venn diagram comparing DEGs in the aged brain of untransplanted- and transplanted mice. 5 genes were common in three groups and 14 genes were common in two out of three groups. (F)Heatmap comparing expression of 19 (5+14) overlapping genes in all 6 groups. (G) Normalized expression levels of 5 genes commonly enriched in the aged groups (A, YA, and AA) in all 6 groups. Data are mean±SEM. (H) Representative image of Iba-1+ cells in the brain of untransplanted (top two rows), young BM-transplanted (middle two rows) and aged BM-transplanted (bottom two rows) young and aged mice. Images were digitally processed and converted for quantification. (I) Quantification of Iba-1+ cell morphology in the cortex of indicated mice. Left columns: Total length of processes. Center columns: number of branch points. Right columns: Ratio of cell body to total cell area. (Horizontal bars represent median values, n=3/group, 3 brain sections/animal were quantified. **p<0.01, ***p<0.001, Student's t-test).

FIG. 8. Circulation-derived myeloid cells adopt a ramified microglia-like morphology upon brain engraftment. (A and B) Flow cytometry gating for analysis of donor chimerism in PB leukocytes (CD45+) (A) and brain myeloid cells (CD45+CD11b+)(B). (C) Representative images of circulation-derived myeloid cells (CDMCs)(Iba-1+GFP−) and host microglia (Iba-1+GFP−) cells and DAPI in the cortex of recipient mice at the indicated time points. Scale bars:50 μm. (D) Representative images of Iba-1+ cells in the cortex of mice treated with control or PLX5622 diet for 21 days. The right panels in each group are enlarged images of the boxed areas in the left panels. Scale bars: 50 m. (E) Iba-1+ cell density in the indicated brain regions was analyzed by microscopy (Mean±SEM, n=3/group, 6 brain slices/animal were quantified. ***p<0.001, Student's t-test).

FIG. 9. Transient treatment with PLX5622 after HSCT does not affect PB myeloid cell populations. (A) Flow cytometry gating for analysis of donor chimerism in PB myeloid cells. (B) Myeloid chimerism in PB was analyzed by flow cytometry at the indicated time point (Horizontal bars represent median values. n=5-6/time point, ns: not significant, Student's t-test). (C) The effect of PLX5622 on PB myeloid cells was analyzed by flow cytometry. Flow cytometry gating for analysis of the indicated cell populations on D38. (D) Quantification of number of the indicated cell populations by flow cytometry (Mean±SEM, n=6/group, ns: not significant, Student's t-test). (E) Representative images of GFP+ and Iba-1+ cells, and DAPI in the cortex of recipient mice at the indicated time points. Scale bars:100 m.

FIG. 10. Baseline and LPS-induced cytokine production in the brains of untransplanted and transplanted mice is comparable. (A) Schematic overview of measurement of cytokines in brain extracts from untransplanted and HSCT+P mice 12 weeks after transplantation. (B) Untransplanted and HSCT+P mice were IP injected with PBS or LPS and then brain homogenates were prepared from those mice 24 h later. Cytokines in brain homogenates were measured using a Luminex assay (Mean±SD, n=4-6/condition, *p<0.05, ***p<0.001, ns: not significant, ND: Not Detected, Student's t-test)

FIG. 11. CDMCs engrafted after HSCT+P are similar to yolk sac-derived microglia-like cells. (A) Gene ontology classes selected from DEGs between microglia and CDMCs. (B) Expression of top 100 DEGs between CDMCs and microglia identified in this study was analyzed in published datasets (Bruttger et al., 2015; Cronk et al., 2018; Lund et al., 2018). (C) Unsupervised hierarchical clustering of CDMCs, microglia (this study) and various microglia-like cells of different origins (Bennett et al., 2018). (Bennett et al., 2018). Untransplanted cultured microglia (Cultured MG, n=3, light green), untransplanted intact microglia (WT MG, n=7, dark brown), Intracranial transplanted (ICT)-P5 microglia (n=4, light brown), ICT-cultured microglia (ICT cultured MG, n=4, dark green), ICT-adult microglia (ICT-adult MG, n=2, dark red), ICT-yolk sac cells (ICT-YS, n=3, orange), ICT-fetal brain cells FB (ICT-FB, n=5, pink), ICT-fetal liver cells (ICT-FL, n=7, red), ICT-BM cells (ICT-BM, n=3, green), ICT-peripheral blood (ICT-Blood, n=3, light green), microglia in this study (Microglia, n=4, light green with dot lines) and CDMCs in this study (CDMCs, n=6, light red with dot lines).

FIG. 12. CDMCs appear to self-renew within the brain. (A and B) Time-course analysis of CD45+CD11b+GFP− and CD45+CD11b+GFP+ cells after HSCT with PLX5622 treatment. The CD45hiCd11b+ gate (pink) is conventionally used to detect perivascular macrophages (Mφ gate). The CD45int/loCD11b+ gate (blue) is widely used to detect microglia (MG gate) (A). Percent GFP+ cells in the brain myeloid cells was analyzed by flow cytometry. (Mean±SEM. n=3-4/time point). (C and D) Flow cytometry gating for analysis of tdTomato expression in PB and brain myeloid cells. Recipient mice were treated with PLX5622 once (C) and twice (D). (E) Representative pictures of tdTomato+, GFP+, and Iba1+ cells. tdTomato+ cells in the cortex on day 132. Yellow arrow heads indicate GFP+tdTomato− cell. White arrows indicate GFP− cells. Scale bars: 50 μm.

FIG. 13. Gating strategies of flow cytometry for isolation of various hematopoietic cells. (A-E) Flow cytometry gating for isolation of HSCs, CMPs, GMPs, and MEPs from bone marrow (A), MDP/CDP from bone marrow (B), BM monocytes (C), Spleen myeloid cells (D) and PB monocytes (E).

FIG. 14. Microglia and CDMCs in the aged brain have similar phenotypes. (A) GFP chimerism of myeloid cells in the brain was analyzed by flow cytometry (Horizontal bars represent mean values. n=4-5/group. ns: not significant. Student's t-test). (B) Representative images of GFP+ and Iba-1+ cells in the brain of transplanted mice. (C) Unsupervised hierarchical clustering of microglia and CDMCs in the brain of young and aged mice. (D) Volcano plot showing DEGs in myeloid cells between young and aged brains. Microglia (left), young bone marrow-derived CDMCs (middle) and aged bone marrow-derived CDMCs (right). (E) Gene ontology analysis of DEGs enriched in A (left), YA (middle), and AA (right). (F) Venn diagram comparing DEGs in the young brain of untransplanted- and transplanted mice. (G) Venn diagram comparing aged myeloid genes and DAM genes (Keren-Shaul et al., 2017). (H) Heatmap representing expression of 22 overlapping genes in all 6 groups.

FIG. 15. HSC-derived CDMCs have similar characteristics to WBM-derived CDMCs. (A) Representative pictures of GFP+ and Iba-1+ cells and DAPI of 4 selected groups. HSC-derived CDMCs are not only the most abundant but also the most microglial-like. Scale bars: 50 μm. (B) Flow cytometry gating for identification of sorted cell-derived CDMCs (GFP+CD45+CD11b+) and WBM-derived CDMCs (Kuo+CD45+CD11b+). (C) PCA plot for microglia (n=8), WBM-derived CDMC (WBM-CDMC, n=6), and HSC-derived CDMC (HSC-CDMC, n=3) using 2,500 most variable genes. Ellipses demarcate 95% confidence interval for assigned clusters.

FIG. 16. Microglia replacement mitigates Purkinje cells degeneration in a mouse model of Gaucher disease (A) Gene mutation and transgene in Psap_{High} and Psap_{low} mice. control (CTRL) and Gaucher disease (GD) mice. (B) Schematic of experimental BMT outline. (C) Representative pictures of Psap+ and Iba-1+ cells in the cerebellum of indicated mice. Scale bars: 100 μm (D) Quantitative analysis of fluorescence intensity of Psap in Iba-1compartment (left) and Iba-1+ cells (right) in the indicated samples. (*p<0.05, ***p<0.001, ns: not significant, ANOVA) (E) Brain extracts from the cerebellum of the indicated groups were analyzed by Western blot for Psap levels. Representative blot is shown. (F) Quantification of blots. Six mice per group were used for quantification (n=3, ###p<0.001 vs CTRL, *p<0.05, ns: not significant, ANOVA). (G) Representative pictures of Calbindin-positive Purkinje cells (arrow heads) in the cerebellum in the indicated samples. ML: molecular layer. GL: granular layer. Scale bars: 50 μm. (H) Quantitative analysis of Calbindin-positive Purkinje cells in the indicated samples (n=3/group, 3 brain slices/animal were quantified, ***p<0.001, ANOVA).

FIG. 17. BMT+P ameliorates astrogliosis in a mouse model of Gaucher disease (A) Representative 1-D plots of droplet digital (dd) PCR assays. ddPCR was performed specific for loci unique to the Y sex chromosome (top:KDM5D, middle: UTY, bottom: SRY) and correlated their signal with an internal reference specific for a bi-allelic autosomal locus. (B) A dilution series with known male and female mouse genomic DNA was prepared and % male in samples was calculated (Mean±SEM). (C) Donor male chimerism in myeloid cells (CD45+CD11b+) of PB (n=10) and brain (n=3) was analyzed by ddPCR in female Psap_{High} mice receiving male Psap_{High} bone marrow. (D to G) Representative pictures of GFAP positive astrogliosis and fluorescence intensity of GFAP in the indicated brain regions. Scale bars: 50 μm. (n=3/group, 3 brain slices/animal were quantified, ***p<0.01, ***p<0.001, ANOVA).

FIG. 18 Microglia replacement ameliorates disease progression in a mouse model of Gaucher disease. (A and B) Motor coordination in the indicated groups was evaluated by the elevated beam test. The beam used was 100 cm in length and 17 mm in width. Number of falling off the beam (A) and time to cross the beam (B) were measured. (C to F) Footprints of the indicated groups were evaluated for stride length of left forelimb (C), right forelimb (D), left hindlimb (E), and right hindlimb (F). (G) Locomotor behavior was assessed by the open field test in the indicated groups. Plot demonstrates area measure, a metric of the number of sharp turns instead of straight-line runs during a 20-minute open field trial. All behavior assays were analyzed statistically by ANOVA. Mean±SEM *p<0.05, **p<0.005, ***p<0.0005, ****p<0.0001. (H) Lifespan of Psap_{low} (green, n=10), Psap_{low}→Psap_{low} (black, n=9), Psap_{High}→Psap_{low} (red, n=7), and Psap_{High}→Psap_{High}, (blue, n=8). (***p<0.001, *p<0.05, ns: not significant, vs Psap_{low} mice, Log rank).

FIG. 19 BMT+P mitigates gait dysfunction in a mouse model of Gaucher disease (A) Examples of footprint assays in the indicated mouse groups. Forepaws were marked with red ink, hind paws with black ink. Stride and base lengths were measured. (B) Footprints of the indicated groups were evaluated for front base and hind base (ANOVA. Mean±SEM *p<0.05, **p<0.005, ***p<0.0005).

FIG. 20. Lentivirus infection of expanded HSC and stable transgene expression following transplantation and incorporation into the brain. (A) Flow cytometry gating for analysis of infection efficiency for expanded mouse HSCs in vitro. (B) HSCs derived from a constitutively GFP− expressing mouse were infected with a lentivirus encoding the dsRed gene and transplanted as described in the BMT/PLX protocol. 7 weeks after transplantation brains were harvested, dissociated, stained and analyzed by flow cytometry which revealed that myeloid cells (CD45/CD11b double positive cells) were GFP and dsRed fluorescent.

FIG. 21. Efficient microglia replacement in the spinal cord following our BMT+PLX protocol. (a) Flow cytometry for total spinal cord demonstrates 90% myeloid cells found to be GFP+ in PLX5622 treated group (b)Histology revealed that the majority of GFP+ cells had attained a microglia like morphology and stained for Iba-1 in lumbar spinal cord

FIG. 22. MRgFUS enhances CDMC delivery into the brain parenchyma after unilateral treatment without toxic preconditioning. Immunocytochemistry for GFP⁺/CD68⁺ CDMCs after unilateral treatment of WT mouse after busulfan-free BMT protocol. FUS treated cerebral hemisphere and non-FUS treated cerebral hemisphere.

### DETAILED DESCRIPTION OF THE EMBODIMENTS

It is an objective of the present invention to provide a new clinically applicable method of glial cell replacement that facilitates engraftment. Aspects of the present invention are based on the discovery that a depletion of the endogenous stem cell niche that facilitates efficient engraftment of hematopoietic stem cells (HSCs) is accomplished by combining the use of an agent that targets the endogenous stem cells, e.g. anti-CD117 antibody, with an agent that enhances the killing of endogenous stem cells by blocking the interaction of CD47 and SIRPα. In particular, the present invention combines this improved selective ablation of endogenous stem cells, in combination with the administration to the recipient of exogenous stem cells, resulting in efficient, long-term engraftment and tolerance.

To facilitate an understanding of the invention, a number of terms are defined below.

Before the present active agents and methods are described, it is to be understood that this invention is not limited to the particular methodology, products, apparatus and factors described, as such methods, apparatus and formulations may, of course, vary. It is also to be understood that the terminology used herein is for the purpose of describing particular embodiments only, and is not intended to limit the scope of the present invention which will be limited only by appended claims.

It must be noted that as used herein and in the appended claims, the singular forms “a,” “and,” and “the” include plural referents unless the context clearly dictates otherwise. Thus, for example, reference to “a drug candidate” refers to one or mixtures of such candidates, and reference to “the method” includes reference to equivalent steps and methods known to those skilled in the art, and so forth.

Unless defined otherwise, all technical and scientific terms used herein have the same meaning as commonly understood by one of ordinary skill in the art to which this invention belongs. All publications mentioned herein are incorporated herein by reference for the purpose of describing and disclosing devices, formulations and methodologies which are described in the publication and which might be used in connection with the presently described invention.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

In the following description, numerous specific details are set forth to provide a more thorough understanding of the present invention. However, it will be apparent to one of skill in the art that the present invention may be practiced without one or more of these specific details. In other instances, well-known features and procedures well known to those skilled in the art have not been described in order to avoid obscuring the invention.

Generally, conventional methods of protein synthesis, recombinant cell culture and protein isolation, and recombinant DNA techniques within the skill of the art are employed in the present invention. Such techniques are explained fully in the literature, see, e.g., Maniatis, Fritsch & Sambrook, Molecular Cloning: A Laboratory Manual (1982); Sambrook, Russell and Sambrook, Molecular Cloning: A Laboratory Manual (2001); Harlow, Lane and Harlow, Using Antibodies: A Laboratory Manual: Portable Protocol No. I, Cold Spring Harbor Laboratory (1998); and Harlow and Lane, Antibodies: A Laboratory Manual, Cold Spring Harbor Laboratory; (1988).

Microglia and Circulation Derived Myeloid Cells (CDMC).

Microglia are the resident immune cells of the brain, and have a myeloid phenotype. They represent 10-20% of all glial cells. The cells may be characterized as one or more of being CD45 positive, CD11b positive, and IBa-1 positive. In contrast to neurons, microglia can proliferate, particularly during infection and injury and in the presence of endogenously produced toxic proteins. Both perivascular and parenchymal microglial cells and macrophages derive from myeloid progenitors. As shown herein, hematopoietic stem cells in the bone marrow can give rise to circulating cells that infiltrate the CNS and differentiate into microglial cells.

Ionized calcium-binding adaptor molecule 1 (Iba1) is a microglial and macrophage-specific calcium-binding protein that is involved with the membrane ruffling and phagocytosis in activated microglia, with a molecular weight of 17,000, and can provide a useful marker for distinguishing microglia and CDMC.

The term CDMC, or brain-resident CDMC may be used herein to refer to cells of a myeloid phenotype, such as microglial cells, that are derived from progenitor cells present in the circulation; and particularly that are derived from exogenously derived hematopoietic stem and progenitor cells, i.e. transplanted cells. Exogenously derived cells may be traced, for example, by expression of a marker of interest that distinguishes from native cell, e.g. expression of a therapeutic gene, presence of a cell surface isotype markers, expression of a fluorescent or bioluminescent marker, and the like.

Microglia are phagocytotic cells, and their ability to phagocytose toxic proteins, pathogens, tumor cells, etc. provides a therapeutic approach for many diseases. Toxic proteins are produced in a variety of brain diseases, such as Alzheimer's disease (b-amyloid), amyotrophic lateral sclerosis (superoxide dismutase 1), and Parkinson's disease (α-synuclein). Microglia are recruited in such conditions, but endogenous microglia are not necessarily efficient at phagocytosis and removal of these toxic proteins from the extracellular environment. These deficiencies may be overcome by the recruitment and infiltration of CDMC. For example, in the case of Alzheimer's disease, increasing the infiltration of blood derived microglial cells enhances the ability to eliminate or prevent the formation of β-amyloid deposits.

Hematopoietic stem-cell transplantation (HSCT) is the transplantation of multipotent hematopoietic stem cells, usually derived from bone marrow, peripheral blood, or umbilical cord blood. It may be autologous, allogeneic, syngeneic or xenogeneic.

The term “recipient” or “host” as used herein refers to a subject receiving transplanted or grafted tissue. These terms may refer to, for example, a subject receiving an administration of donor bone marrow, or other sources of HSC.

The term “donor” as used herein refers to a subject from whom tissue is obtained to be transplanted or grafted into a recipient or host. For example, a donor may be a subject from whom bone marrow, mobilized peripheral blood stem cells, etc. to be administered to a recipient or host is derived.

The term “chimerism” as used herein refers to a state in which one or more cells from a donor are present and functioning in a recipient or host. Recipient tissue exhibiting “chimerism” may contain donor cells only (complete chimerism), or it may contain both donor and host cells (mixed chimerism). “Chimerism” as used herein may refer to either transient or stable chimerism. The data presented herein demonstrate that the methods of the invention provide for high level chimerism, e.g. at least about 25% donor-derived brain-resident CDMC following the transplantation procedure, at least about 35%, at least about 50%, at least about 65%, at least about 75%, at least about 85%, at least about 90%, or more. The presence of donor-derived cells in the brain increases following the second conditioning step, and reaches a high level of chimerism after about 4, about 5, about 6, about 7, about 8 weeks. The level of chimerism stay high for at least about 4 weeks, at least about 8 weeks, at least about 12 week, at least about 16 week, or longer.

Hematopoietic Stem Cells.

The term stem cell is used herein to refer to a mammalian cell that has the ability both to self-renew, and to generate differentiated progeny (see Morrison et al. (1997) Cell 88:287-298). Generally, stem cells also have one or more of the following properties: an ability to undergo asynchronous, or symmetric replication, that is where the two daughter cells after division can have different phenotypes; extensive self-renewal capacity; capacity for existence in a mitotically quiescent form; and clonal regeneration of all the tissue in which they exist, for example the ability of hematopoietic stem cells to reconstitute all hematopoietic lineages. The cells of interest are typically mammalian, where the term refers to any animal classified as a mammal, including humans, domestic and farm animals, and zoo, laboratory, sports, or pet animals, such as dogs, horses, cats, cows, mice, rats, rabbits, etc. Preferably, the mammal is human.

For engraftment purposes, a composition comprising stem cells, including, without limitation, hematopoietic stem cells, is administered to a patient. Such methods are well known in the art. The stem cells are optionally, although not necessarily, purified. Abundant reports explore various methods for purification of stem cells and subsequent engraftment, including flow cytometry; an isolex system (Klein et al. (2001) Bone Marrow Transplant. 28(11):1023-9; Prince et al. (2002) Cytotherapy 4(2):137-45); immunomagnetic separation (Prince et al. (2002) Cytotherapy 4(2):147-55; Handgretinger et al. (2002) Bone Marrow Transplant. 29(9):731-6; Chou et al. (2005) Breast Cancer. 12(3):178-88); and the like. Each of these references is herein specifically incorporated by reference, particularly with respect to procedures, cell compositions and doses for hematopoietic stem cell transplantation.

Hematopoietic stem cells can be obtained by harvesting from bone marrow or from peripheral blood. Bone marrow is generally aspirated from the posterior iliac crests while the donor is under either regional or general anesthesia. Additional bone marrow can be obtained from the anterior iliac crest. A dose of 5×10⁷, 10⁸, 2×10⁸ marrow mononuclear cells per kilogram is generally considered desirable to establish engraftment in autologous and allogeneic marrow transplants, respectively. Bone marrow can be primed with granulocyte colony-stimulating factor (G-CSF; filgrastim [Neupogen]) to increase the stem cell count.

In some embodiments the donor cells are autologous, including without limitation genetically corrected autologous cells. In some embodiments the donor cells are mobilized peripheral blood cells; in other embodiments the donor cells are bone marrow cells. In some embodiments the donor cells are enriched for expression of CD34, e.g. by art recognized methods such as the cliniMACS® system, by flow cytometry, etc. Cell populations single enriched for CD34 may be from about 50% up to about 90% CD34⁺ cells. Alternatively cell populations may be tandemly selected for expression of CD34 and CD90, which cell populations may be highly purified, e.g. at least about 85% CD34⁺CD90⁺ cells, at least about 90% CD34⁺CD90⁺ cells, at least about 95% CD34⁺CD90⁺ cells and may be up to about 99% CD34⁺CD90⁺ cells or more. Alternatively unmanipulated bone marrow or mobilized peripheral blood populations are used.

Mobilization of stem cells from the bone marrow into peripheral blood by cytokines such as G-CSF or GM-CSF has led to the widespread adoption of peripheral blood progenitor cell collection by apheresis for hematopoietic stem cell transplantation. The dose of G-CSF used for mobilization is 10 μg/kg/day. In autologous donors who are heavily pretreated, however, doses of up to 40 μg/kg/day can be given. Mozobil may be used in conjunction with G-CSF to mobilize hematopoietic stem cells to peripheral blood for collection.

Current guidelines indicate that the minimum dose required for engraftment is 1-2×10⁶ CD34⁺ cells/kg body weight for autologous and allogeneic transplants. Higher doses can result in better engraftment, but doses in the range of greater than 8×10⁶ have been associated with increased risk of extensive GVHD.

The cells which are employed may be fresh, frozen, or have been subject to prior culture. They may be fetal, neonate, adult, etc. Hematopoietic stem cells may be obtained from fetal liver, bone marrow, blood, particularly G-CSF or GM-CSF mobilized peripheral blood, or any other conventional source. Cells may also be differentiated in vitro from pluripotent cells, e.g. iPSC cells. Cells for engraftment are optionally isolated from other cells, where the manner in which the stem cells are separated from other cells of the hematopoietic or other lineage is not critical to this invention. If desired, a substantially homogeneous population of stem or progenitor cells may be obtained by selective isolation of cells free of markers associated with differentiated cells, while displaying epitopic characteristics associated with the stem cells.

Autologous transplantation can be used, for example, where the autologous cells are genetically modified to provide additional therapeutic benefits, or where there is a deficiency in endogenous glial cells that is to be rectified.

Allogeneic transplantation refers to the use of stem cells from a donor source other than the subject. The source of donated stem cells (the donor) may be genetically related or unrelated to the recipient. The degree of HLA match between the donor and the recipient is an important factor in these transplants; well-matched transplants decrease the risk of graft rejection and graft versus host disease (GVHD). ABO-mismatched donors are acceptable donors for bone marrow transplantation, but they may have ABO-mismatch-related complications. Immunocompetent donor T cells may be removed using a variety of methods to reduce or eliminate the possibility that graft versus host disease (GVHD) will develop.

Where the donor is allogeneic to the recipient, the HLA type of the donor and recipient may be tested for a match. Traditionally, the loci critical for matching are HLA-A, HLA-B, and HLA-DR. HLA-C and HLA-DQ are also now considered when determining the appropriateness of a donor. A completely matched sibling donor is generally considered the ideal donor. For unrelated donors, a complete match or a single mismatch is considered acceptable for most transplantation, although in certain circumstances, a greater mismatch is tolerated. Preferably matching is both serologic and molecular. Where the donor is umbilical cord blood the degree of tolerable HLA disparity is much greater, and a match of 3-4 out of the 6 HLA-A, HLA-B and HLA-DRB1 antigens is sufficient for transplantation. Immunocompetent donor T cells may be removed using a variety of methods to reduce or eliminate the possibility that graft versus host disease (GVHD) will develop.

Cells may be genetically altered in order to introduce genes useful in the differentiated cell, e.g. repair of a genetic defect in an individual, selectable marker, etc., or genes useful in selection against undifferentiated ES cells. Cells may also be genetically modified to enhance survival, control proliferation, and the like. Cells may be genetically altering by transfection or transduction with a suitable vector, homologous recombination, or other appropriate technique, so that they express a gene of interest. Genes of interest include, for example, wild-type protein versions of proteins that are deficient in different lysosomal storage disorders, e.g., beta-glucosidase in Gauchers Disease, cytokines, e.g. anti-inflammatory cytokines or other immunosuppressive molecules; agents effective in the treatment of brain cancer, such as glioblastoma, e.g. enzymes that activate prodrugs, secreted factors that can slow or interrupt cancer stem cell self-renewal or induce cancer stem cells to differentiate immune checkpoint inhibitors, targeted antibodies or nanobodies e.g. specific for EGFRvIII, or expression of a chimeric antigen receptor such that the microgial cells act as CAR macrophages with targeted killing of brain cancer cells; secretion of chaperone proteins for alleviation of Alzheimer's disease. In such embodiments, a selectable marker can be introduced, to provide for greater purity of the desired differentiating cell. Cells may be genetically altered using vector containing supernatants over an 8-16 h period, and then exchanged into growth medium for 1-2 days. Genetically altered cells are selected using a drug selection agent such as puromycin, G418, or blasticidin, and then recultured.

The cells of this invention can also be genetically altered in order to enhance their ability to be involved in tissue regeneration, or to deliver a therapeutic gene to a site of administration. A vector is designed using the known encoding sequence for the desired gene, operatively linked to a promoter that is constitutive, pan-specific, specifically active in a differentiated cell type, etc. Suitable inducible promoters are activated in a desired target cell type, either the transfected cell, or progeny thereof. By transcriptional activation, it is intended that transcription will be increased above basal levels in the target cell by at least about 100 fold, more usually by at least about 1000 fold. Various promoters are known that are induced in different cell types.

Many vectors useful for transferring exogenous genes into target mammalian cells are available. The vectors may be episomal, e.g. plasmids, virus derived vectors such cytomegalovirus, adenovirus, etc., or may be integrated into the target cell genome, through homologous recombination or random integration, e.g. retrovirus derived vectors such MMLV, HIV-1, ALV, etc. For modification of stem cells, lentiviral vectors are preferred. Lentiviral vectors such as those based on HIV or FIV gag sequences can be used to transfect non-dividing cells, such as the resting phase of human stem cells. Combinations of retroviruses and an appropriate packaging line may also find use, where the capsid proteins will be functional for infecting the target cells. Usually, the cells and virus will be incubated for at least about 24 hours in the culture medium. The cells are then allowed to grow in the culture medium for short intervals in some applications, e.g. 24-73 hours, or for at least two weeks, and may be allowed to grow for five weeks or more, before analysis. Commonly used retroviral vectors are “defective”, i.e. unable to produce viral proteins required for productive infection. Replication of the vector requires growth in the packaging cell line. The vectors may include genes that must later be removed, e.g. using a recombinase system such as Cre/Lox, or the cells that express them destroyed, e.g. by including genes that allow selective toxicity such as herpesvirus TK, bcl-xs, etc.

A “patient” for the purposes of the present invention includes both humans and other animals, particularly mammals, including pet and laboratory animals, e.g. mice, rats, rabbits, etc. Thus the methods are applicable to both human therapy and veterinary applications. In one embodiment the patient is a mammal, preferably a primate. In other embodiments the patient is human.

Additional Terms.

The terms “treatment”, “treating”, “treat” and the like are used herein to generally refer to obtaining a desired pharmacologic and/or physiologic effect. The effect can be prophylactic in terms of completely or partially preventing a disease or symptom(s) thereof and/or may be therapeutic in terms of a partial or complete stabilization or cure for a disease and/or adverse effect attributable to the disease. The term “treatment” encompasses any treatment of a disease in a mammal, particularly a human, and includes: (a) preventing the disease and/or symptom(s) from occurring in a subject who may be predisposed to the disease or symptom but has not yet been diagnosed as having it; (b) inhibiting the disease and/or symptom(s), i.e., arresting their development; or (c) relieving the disease symptom(s), i.e., causing regression of the disease and/or symptom(s). Those in need of treatment include those already inflicted (e.g., those with cancer, those with an infection, etc.) as well as those in which prevention is desired (e.g., those with increased susceptibility to cancer, those with an increased likelihood of infection, those suspected of having cancer, those suspected of harboring an infection, etc.).

### Methods of Treatment

The initial step of the glial cell replacement method provides for HSCT. Various methods known in the art can be used for this purpose. As is known in the art, a recipient is first subjected to a “conditioning” regimen, which depletes native HSC from the body to allow for an open “niche” in which the exogenous stem cells can grow. The preparative or conditioning regimen is a critical element in hematopoietic stem cell transplantation (HSCT). The purpose of the preparative regimen is to provide immunosuppression sufficient to prevent rejection of the transplanted graft. These goals have traditionally been achieved by delivering maximally tolerated doses of multiple chemotherapeutic agents with nonoverlapping toxicities (with or without radiation). In some embodiments, a conditioning regimen utilizing agents that can cross the blood brain barrier and clear microgial niches is preferred. In other embodiments, a less toxic, for example antibody-based regimen is preferred.

Nonmyeloablative preparative regimens include targeted therapy by administration of an antibody that specifically depletes HSC. Many such therapies utilize antibodies specific for CD117 (c-kit), including the methods disclosed, and herein specifically incorporated by reference, in U.S. Pat. Nos. 10,072,091; 10,406,179; US Patent Application US 2018-0214524 A1; the methods of using AMG-191 disclosed in Blood (2019) 134 (Supplement_1): 800; drug-conjugated CD117 antibodies with amanitin (MGTA-117), mertansine, etc.

In embodiments utilizing nonmyeloablative preparative regimens in the absence of busulfan, including targeted therapy by administration of an antibody that specifically depletes HSC, it may be desirable to include an MRI-guided focused ultra-sound regimen to increase permeability of the BBB to circulating cells. This regimen can be combined with a microglial conditioning agent, such as brain penetrant inhibitors of CSF1R. For example, BBB disruption with focused ultrasound using repeat exposures of the target region in the brain over multiple minutes can be used. The nature of the ultrasound exposure has an influence on the size and type of molecules that will pass through the BBB into brain parenchyma. See, for example, Chopra et al. ACS Chem Neurosci. 2010; 1(5):391-8; Shen et al. 2017; 26(7):1235-46; Burgess et al. PLoS One. 2011; 6(11):e27877; Leinenga and Gotz, Sci Transl Med. 2015; 7(278):278ra33, each herein specifically incorporated by reference.

Myeloablative regimens can be classified as radiation-containing or non-radiation-containing regimens, therapies that were developed by escalating the dose of radiation or of a particular drug to the maximally tolerated dose. Drugs with nonoverlapping toxicities have been used in an effort to avoid synergistic injury to a particular organ. Total-body irradiation and one or both of cyclophosphamide and busulfan are commonly used myeloablative therapies.

Total-body irradiation regimens typically fractionate the radiation and administer the total dose over several days (a strategy termed fractionated total-body irradiation [FTBI]), which helps to decrease toxicity and increase tolerability. Partial lung shielding is included in an effort to reduce the potential for irreversible lung injury. The maximally tolerated dose of total-body irradiation is approximately 1500 cGy. Higher doses produce excessive nonhematologic toxicity, primarily to the lungs, but also to other organs, including the heart. Commonly used radiation-containing preparative regimens are as follows:
FTBI at 1200 cGy and cyclophosphamide at 120 mg/kg
FTBI at 1320 cGy and etoposide at 60 mg/kg
FTBI at 1320 cGy, etoposide at 60 mg/kg, and cyclophosphamide at 60 mg/kg
FTBI at 1200 cGy and melphalan at 200 mg/kg
FTBI at 1200 cGy, etoposide at 60 mg/kg, and cyclophosphamide (for autologous) at 100 mg/kg

Non-radiation-containing preparative regimens have been developed in which total-body irradiation is replaced with additional chemotherapeutic agents. Commonly used non-radiation-containing preparative regimens are as follows: Busulfan at 16 mg/kg and cyclophosphamide at 120 mg/kg; Busulfan at 16 mg/m² and etoposide at 60 mg/m²; cyclophosphamide at 6-7.2 g/m², carmustine at 300-500 mg/m², and etoposide at 600-2400 mg/m²; cyclophosphamide at 7.2 g/m², carmustine at 600 mg/m², etoposide at 1200 mg/m², and cisplatin at 150 mg/m²; carmustine at 300 mg/m², etoposide at 400-800 mg/m², cytarabine at 800-1600 mg/m², and melphalan at 140 mg/m².

With nonmyeloablative regimens, doses of chemotherapeutic drugs and radiation that are substantially lower than those of myeloablative regimens. These regimens are immunosuppressive but not myeloablative. Reduced-intensity regimens can range in intensity from myeloablative to nonmyeloablative, and involve drugs such as fludarabine, melphalan, antithymocyte globulin, and busulfan. Such regimens also reduce acute and chronic toxicity compared with myeloablative regimens, although the incidence of GVHD is comparable with that of myeloablative regimens.

The infusion of the HSC, e.g. bone marrow, peripheral blood progenitor cells (PBPCs), in vitro modified cells, etc. is a relatively simple process that is performed at the bedside. The cell product is generally used fresh and is infused through a central vein over a period of several hours. Autologous products are almost always cryopreserved; they are thawed at the bedside and infused rapidly over a period of several minutes. The hematopoietic stem cells engraft within the bone marrow cavity by homing like mechanisms. Vascular cell adhesion molecule-1, heparan sulphate, and stromal cell-derived factor-1 and its receptor (CXCR4) appear to play roles in this process. The dose of HSC is at least about 10⁵ CD34⁺ cells/kg body weight, at least about 0.5×10⁶, at least about 10⁶, and up to about 2.5×10⁶, 5×10⁶, 7.5×10⁶, 10⁷ CD34⁺ cells/kg body weight.

A microglial cell conditioning regimen is applied following the HSCT procedure. Conditioning agents of interest are brain penetrant inhibitors of colony stimulating factor (CSF). In some embodiments the microglial conditioning agent is an inhibitor of CSF1 receptor (CSF1R). CSF1R-mediated signaling is crucial for the differentiation and survival of the mononuclear phagocyte system and macrophages in particular. CSF1R belongs to the type III protein tyrosine kinase receptor family, and binding of CSF1 or the more recently identified ligand, IL-34, induces homodimerization of the receptor and subsequent activation of receptor signaling. Targeting CSF1R signaling reduces the population of endogenous CDMC.

A number of small molecule CSF1R inhibitors are known in the art and have been tested in human clinical trials. For example, and without limitation, such agents include Pexidartinib (PLX3397, PLX108-01) (Plexxikon); ARRY-382 (Array BioPharma); BLZ945 (Novartis); PLX7486 (Plexxikon); JNJ-40346527 (Johnson& Johnson); PLX5622 (Plexxikon).

The interval of time between infusion of HSC and administration of the microglial cell conditioning agent may be at least about 3 days, at least about 7 days, at least about 10 days, at least about 14 days, at least about 16 days, at least about 21 days, at least about 25 days, at least 28 days. The interval may be not more than about 48 days. In some embodiments the interval of time is from about 14 to about 21 days, or from about 14 to about 28 days.

The conditioning agent is administered daily, twice daily, every other day, every third day, etc. for a period of time sufficient to effect the desired reduction in numbers of brain resident microglial cells, for at least 1 day, 2 days, 3, 4, 5, 6, 7, 8 or more days. In some embodiments from 4-10 days is sufficient.

For ablation, the microglial cell conditioning agents are formulated in a pharmaceutical composition. The exact dose will depend on the purpose of the treatment, and will be ascertainable by one skilled in the art using known techniques (e.g., Ansel et al., Pharmaceutical Dosage Forms and Drug Delivery; Lieberman, Pharmaceutical Dosage Forms (vols. 1-3, 1992), Dekker, ISBN 0824770846, 082476918X, 0824712692, 0824716981; Lloyd, The Art, Science and Technology of Pharmaceutical Compounding (1999); and Pickar, Dosage Calculations (1999)). As is known in the art, adjustments for patient condition, systemic versus localized delivery, as well as the age, body weight, general health, sex, diet, time of administration, drug interaction and the severity of the condition may be necessary, and will be ascertainable with routine experimentation by those skilled in the art. The microglial cell conditioning agent is provided in a dose that depletes endogenous microglial cells by at least 10-fold, at least 100-fold, at least 1000-fold, at least 100,000-fold or more. The effective dose will depend on the individual and the specific agent, but will generally be at least about 1 μg/kg body weight, 5 μg/kg body weight, at least about 10 μg/kg, at least about 25 μg/kg, at least about 50 μg/kg, at least about 100 μg/kg, at least about 250 μg/kg, at least about 500 μg/kg, at least about 1 mg/kg, and up to about 2.5 mg/kg, up to about 5 mg/kg, up to about 7.5 mg/kg, up to about 10 mg/kg, up to about 15 mg/kg, up to about 25 mg/kg, up to about 50 mg/kg, up to about 100 mg/kg.

The administration of the agents can be done in a variety of ways as discussed above, including, but not limited to, orally, subcutaneously, intravenously, intranasally, transdermally, intraperitoneally, intramuscularly, intrapulmonary, vaginally, rectally, or intraocularly.

In one embodiment, the pharmaceutical compositions are in a water soluble form, such as being present as pharmaceutically acceptable salts, which is meant to include both acid and base addition salts. “Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. “Pharmaceutically acceptable base addition salts” include those derived from inorganic bases such as sodium, potassium, lithium, ammonium, calcium, magnesium, iron, zinc, copper, manganese, aluminum salts and the like. Particularly useful are the ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine.

The pharmaceutical compositions may also include one or more of the following: carrier proteins such as serum albumin; buffers; fillers such as microcrystalline cellulose, lactose, corn and other starches; binding agents; sweeteners and other flavoring agents; coloring agents; and polyethylene glycol.

The pharmaceutical compositions can be administered in a variety of unit dosage forms depending upon the method of administration. For example, unit dosage forms suitable for oral administration include, but are not limited to, powder, tablets, pills, capsules and lozenges. It is recognized that compositions of the invention when administered orally, should be protected from digestion. This is typically accomplished either by complexing the molecules with a composition to render them resistant to acidic and enzymatic hydrolysis, or by packaging the molecules in an appropriately resistant carrier, such as a liposome or a protection barrier. Means of protecting agents from digestion are well known in the art.

The compositions for administration will commonly comprise an antibody or other ablative agent dissolved in a pharmaceutically acceptable carrier, preferably an aqueous carrier. A variety of aqueous carriers can be used, e.g., buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, e.g., sodium acetate, sodium chloride, potassium chloride, calcium chloride, sodium lactate and the like. The concentration of active agent in these formulations can vary widely, and will be selected primarily based on fluid volumes, viscosities, body weight and the like in accordance with the particular mode of administration selected and the patient's needs (e.g., Remington's Pharmaceutical Science (15th ed., 1980) and Goodman & Gillman, The Pharmacological Basis of Therapeutics (Hardman et al., eds., 1996)).

The compositions containing conditioning agents, e.g. CSF-1R inhibitors, antibodies, etc. can be administered for therapeutic treatment. Compositions are administered to a patient in an amount sufficient to substantially ablate targeted endogenous stem cells, as described above. An amount adequate to accomplish this is defined as a “therapeutically effective dose.” Single or multiple administrations of the compositions may be administered depending on the dosage and frequency as required and tolerated by the patient. The particular dose required for a treatment will depend upon the medical condition and history of the mammal, as well as other factors such as age, weight, gender, administration route, efficiency, etc.

### Conditions for Treatment

The indications for glial cell replacement vary according to disease categories and are influenced by factors such as cytogenetic abnormalities, response to prior therapy, patient age and performance status, disease status (remission vs relapse), disease-specific prognostic factors, availability of a suitable graft source, time of referral, and time to transplant.

Embodiments of the invention include transplantation into a patient suffering from a neurologic disorder, where exogenous stem cells of a normal phenotype are transplanted into the patient, for example for the treatment of lysosomal storage diseases.

The lysosomal storage diseases (LSDs) comprise a heterogeneous group of almost 50 disorders that are caused by genetic defects in a lysosomal acid hydrolase, receptor, activator protein, membrane protein, or transporter, causing lysosomal accumulation of substrates that are specific to each disorder. The accumulation is progressive, ultimately causing deterioration of cellular and tissue function. Many disorders affect the central nervous system (CNS) and most patients have a decreased lifespan and significant morbidity. The LSDs are often categorized according to the type of substrate stored (i.e., mucopolysaccharidoses, oligosaccharidoses, sphingolipidoses, gangliosidoses, etc.). Gaucher disease is of particular interest.

For treatment of neurologic diseases, including without limitation Gaucher disease, allogeneic or autologous HSC can be transplanted to correct the patient enzyme deficiency. Autologous HSC may be genetically modified ex vivo to correct the deficiency. Allogeneic HSC without the enzyme deficiency can be used. Depending on the level of deficiency,

Most lysosomal proteins are the products of housekeeping genes expressed throughout the body, but storage occurs only in those cells with an available substrate (e.g., GM₂ ganglioside is present predominantly in the CNS and deficiency of hexosaminidase A, which acts on the GM₂ ganglioside and can be measured in the blood, causes Tay Sachs disease, a CNS condition). In all cases, the diagnosis can be established by specific enzyme assays and by mutational analysis.

Although the signs and symptoms vary from disease to disease in this group, symptoms occur in each case because of an enzyme deficiency that inhibits the ability of the lysosomes present in each of the body's cells to perform their normal function. The lysosomes function as the primary digestive units within cells. Their function is to break down complex components into simpler ones. Each cell has hundreds of lysosomes that degrade complex cellular components such as proteins (substrates) into simpler components. When this process does not take place, the substrate begins to accumulate in the cells. That is why these diseases are called “storage diseases”. The symptoms of lysosomal storage disorders are generally progressive over a period of time.

Batten Disease: Batten disease is the juvenile form of a group of progressive neurological disorders known as neuronal ceroid lipofuscinoses (NCL). It is characterized by the accumulation of a fatty substance (lipopigment) in the brain, as well as in tissue that does not contain nerve cells. Batten disease is marked by rapidly progressive vision failure (optic atrophy) and neurological disturbances, which may begin before eight years of age. Occurring mostly in families of Northern European Scandinavian ancestry, the disorder affects the brain and may cause deterioration of both intellect and neurological functions.

Gaucher disease is the most common type of lysosomal storage disorder. It is a sphingolipidosis, resulting from glucocerebrosidase deficiency, causing deposition of glucocerebroside and related compounds. Symptoms and signs vary by type but are most commonly hepatosplenomegaly or central nervous system changes. Diagnosis is by DNA analysis and/or enzyme analysis of white blood cells. Treatment is enzyme replacement with glucocerebrosidase.

There are 3 types of Gaucher disease, which vary in epidemiology, enzyme activity, and manifestations. ERT is effective for type 1 disease affecting primarily non-CNS organs. Importantly, however, ERT is ineffective for type 2 and 3 disease, which are characterized by dominant CNS involvement and which find particular benefit from the methods disclosed herein.

Type II (acute neuronopathic) has onset occurs during infancy, with progressive neurologic deterioration. Type III Gaucher disease (subacute neuronopathic) falls between types I and II in incidence, enzyme activity, and clinical severity. Onset occurs at any time during childhood.

Diagnosis of Gaucher disease is by DNA analysis and/or enzyme analysis of white blood cells. Carriers are detected, and types are distinguished by mutation analysis. Although biopsy is unnecessary, Gaucher cells—lipid-laden tissue macrophages in the liver, spleen, lymph nodes, bone marrow, or brain that have a wrinkled tissue-paper appearance—are diagnostic.

Treatment with the methods disclosed herein may be combined with enzyme replacement with IV glucocerebrosidase for peripheral (i.e. non-CNS) manifestations of disease. The enzyme is modified for efficient delivery to lysosomes. Patients receiving enzyme replacement require routine hemoglobin and platelet monitoring, routine assessment of spleen and liver volume by CT or MRI, and routine assessment of bone disease by skeletal survey, dual-energy x-ray absorptiometry scanning, or MRI. Miglustat (100 mg orally 3 times a day), a glucosylceramide synthase inhibitor, reduces glucocerebroside concentration (the substrate for glucocerebrosidase) and is an alternative for patients unable to receive enzyme replacement. Eliglustat (84 mg orally once a day or 2 times a day), another glucosylceramide synthase inhibitor, also reduces glucocerebroside concentration.

GM2-Gangliosidosis Type I (Tay Sachs Disease): Two main forms of Tay Sachs disease exist: the classic or infantile form and the late-onset form. In individuals with infantile Tay Sachs disease, symptoms typically first appear between three and five months of age. These may include feeding problems, general weakness (lethargy), and an exaggerated startle reflex in response to sudden loud noises. Motor delays and mental deterioration are progressive. In individuals with the late-onset form, symptoms may become apparent anytime from adolescence through the mid-30s. The infantile form often progresses rapidly, resulting in significant mental and physical deterioration. A characteristic symptom of Tay Sachs disease, which occurs in 90 percent of cases, is the development of cherry red spots in the backs of the eyes. Symptoms of late-onset Tay Sachs disease vary widely from case to case. This disorder progresses much more slowly than the infantile form.

GM2-Gangliosidosis Type II (Sandhoff Disease): The first symptoms of Sandhoff disease typically begin between the ages of three and six months. The disease is clinically indistinguishable from GM2-Gangliosidosis Type I.

In each case, lysosomal storage diseases are caused by an inborn error of metabolism that results in the absence or deficiency of an enzyme, leading to the inappropriate storage of material in various cells of the body. Most lysosomal storage disorders are inherited in an autosomal recessive manner.

Prenatal diagnosis is possible for all lysosomal storage disorders. Early detection of lysosomal storage diseases, whether before birth or as soon as possible afterward, is important because when therapies are available, either for the disease itself or for associated symptoms, they may significantly limit the long-term course and impact of the disease.

In some embodiments, the microglial cell replacement is used to provide therapeutic proteins and cell activity for the treatment of cancer. The terms “cancer,” “neoplasm,” and “tumor” are used interchangeably herein to refer to cells which exhibit autonomous, unregulated growth, such that they exhibit an aberrant growth phenotype characterized by a significant loss of control over cell proliferation. Cells of interest for detection, analysis, or treatment in the present application include precancerous (e.g., benign), malignant, pre-metastatic, metastatic, and non-metastatic cells. Cancers of virtually every tissue are known. The phrase “cancer burden” refers to the quantum of cancer cells or cancer volume in a subject. Reducing cancer burden accordingly refers to reducing the number of cancer cells or the cancer volume in a subject. The term “cancer cell” as used herein refers to any cell that is a cancer cell or is derived from a cancer cell e.g. clone of a cancer cell.

The “pathology” of cancer includes all phenomena that compromise the well-being of the patient. This includes, without limitation, abnormal or uncontrollable cell growth, metastasis, interference with the normal functioning of neighboring cells, release of cytokines or other secretory products at abnormal levels, suppression or aggravation of inflammatory or immunological response, neoplasia, premalignancy, malignancy, invasion of surrounding or distant tissues or organs, such as lymph nodes, etc.

As used herein, the terms “cancer recurrence” and “tumor recurrence,” and grammatical variants thereof, refer to further growth of neoplastic or cancerous cells after diagnosis of cancer. Particularly, recurrence may occur when further cancerous cell growth occurs in the cancerous tissue. “Tumor spread,” similarly, occurs when the cells of a tumor disseminate into local or distant tissues and organs; therefore tumor spread encompasses tumor metastasis. “Tumor invasion” occurs when the tumor growth spread out locally to compromise the function of involved tissues by compression, destruction, or prevention of normal organ function.

As used herein, the term “metastasis” refers to the growth of a cancerous tumor in an organ or body part, which is not directly connected to the organ of the original cancerous tumor. Metastasis will be understood to include micrometastasis, which is the presence of an undetectable amount of cancerous cells in an organ or body part which is not directly connected to the organ of the original cancerous tumor. Metastasis can also be defined as several steps of a process, such as the departure of cancer cells from an original tumor site, and migration and/or invasion of cancer cells to other parts of the body.

Brain cancers. Primary brain tumors are a diverse group of diseases. Brain tumors are classified according to histology, but tumor location and extent of spread are also important factors that affect treatment and prognosis. Immunohistochemical analysis, cytogenetic and molecular genetic findings, and measures of proliferative activity are increasingly used in tumor diagnosis and classification.

Tumors are classically categorized as infratentorial, supratentorial, parasellar, or spinal. Immunohistochemical analysis, cytogenetic and molecular genetic findings, and measures of proliferative activity are increasingly used in tumor diagnosis and classification and will likely affect classification and nomenclature in the future.

Infratentorial (posterior fossa) tumors include the following: Cerebellar astrocytomas (most commonly pilocytic, but also fibrillary and less frequently, high grade); Medulloblastomas (including classic, desmoplastic/nodular, extensive nodularity, anaplastic, or large cell variants); Ependymomas (papillary, clear cell, tanycytic, or anaplastic); Brain stem gliomas (typically diffuse intrinsic pontine gliomas and focal, tectal, and exophytic cervicomedullary gliomas are most frequently pilocytic astrocytomas); Atypical teratoid/rhabdoid tumors; Choroid plexus tumors (papillomas and carcinomas); Rosette-forming glioneuronal tumors of the fourth ventricle.

Supratentorial tumors include the following: Low-grade cerebral hemispheric astrocytomas (grade I [pilocytic] astrocytomas or grade II [diffuse] astrocytomas); High-grade or malignant astrocytomas (anaplastic astrocytomas and glioblastoma [grade III or grade IV]); Mixed gliomas (low- or high-grade); Oligodendrogliomas (low- or high-grade); Cerebral neuroblastomas and pineoblastomas; Atypical teratoid/rhabdoid tumors; Ependymomas (anaplastic or RELA fusion-positive); Meningiomas (grades I, II, and III); Choroid plexus tumors (papillomas and carcinomas); Tumors of the pineal region (pineocytomas, pineoblastomas, pineal parenchymal tumors of intermediate differentiation, and papillary tumors of the pineal region), and germ cell tumors; Neuronal and mixed neuronal glial tumors (gangliogliomas, desmoplastic infantile astrocytoma/gangliogliomas, dysembryoplastic neuroepithelial tumors, and papillary glioneuronal tumors); other low-grade gliomas (including subependymal giant cell tumors and pleomorphic xanthoastrocytoma); and metastasis (rare) from extraneural malignancies.

Parasellar tumors include the following: Craniopharyngiomas; Diencephalic astrocytomas (central tumors involving the chiasm, hypothalamus, and/or thalamus) that are generally low-grade (including astrocytomas, grade I [pilocytic] or grade II [diffuse]); Germ cell tumors (germinomas or nongerminomatous).

Spinal cord tumors include the following: Low-grade cerebral hemispheric astrocytomas (grade I [pilocytic] astrocytomas or grade∥[diffuse] astrocytomas); High-grade or malignant astrocytomas (anaplastic astrocytomas and glioblastoma [grade III or grade IV]); Gangliogliomas; Ependymomas (often myxopapillary).

### Experimental

### Example 1

### Highly Efficient, Non-Genetic Microglia Replacement Reveals Ageing-Associated Microglia Signature is Induced by Microenvironment

Bone marrow transplantation (HSCT) allows engraftment of circulation-derived myeloid cells (CDMCs) into the brain in genetically unmodified mice but yields variable chimerism. Here, we show that a combination of HSCT followed by pharmacological inhibition of the CSF1R pathway leads to near complete replacement of microglia with CDMCs throughout the entire brain. Our results indicate that the high chimerism stems from enhanced regenerative capacity of brain-resident CDMCs following CSF1R inhibition, rather than from continued influx from the circulation. Unexpectedly, purified hematopoietic stem cells had the highest microgliogenic potential upon transplantation. Young CDMCs incorporated into the aged brain readily acquired aged properties demonstrating that the brain environment imposes a dominant aging phenotype on brain myeloid cells. Our results suggest that the widely observed disease-associated microglia may represent an adaptive response to the pathological environment, rather than a cell-intrinsic mechanism of aging or disease progression. These methods provide a platform for novel microglia-replacement based therapies.

Here, we sought to better characterize the engraftment of CDMCs into the brain in order to improve the efficiency of microglia replacement in non-genetic mouse models for investigative and potential therapeutic use.

### Results

Microglia Replacement Following HSCT is Slow, Inefficient and Highly Variable.

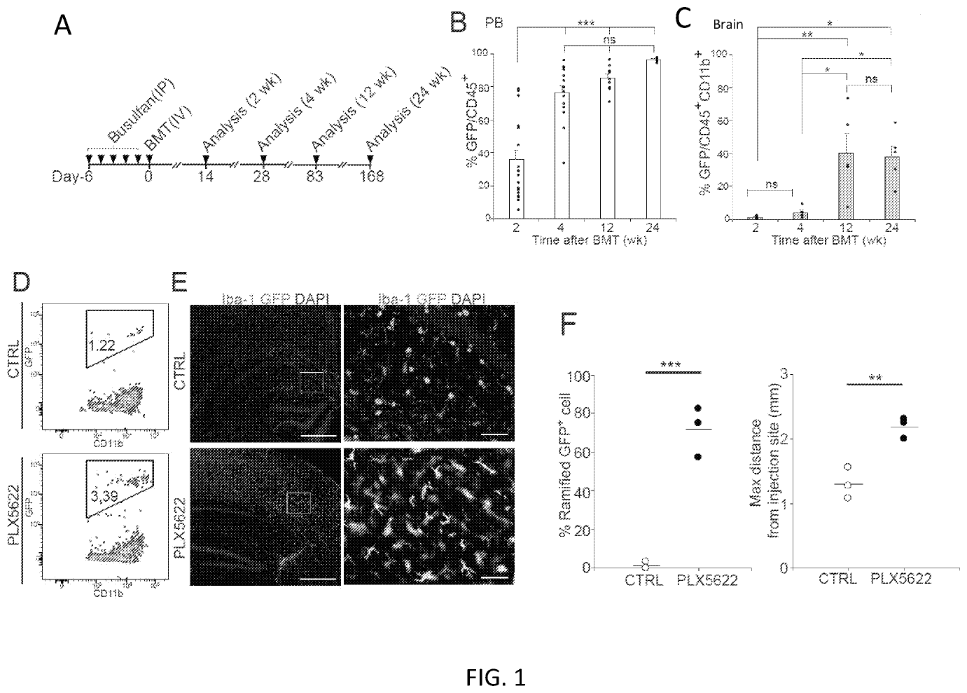

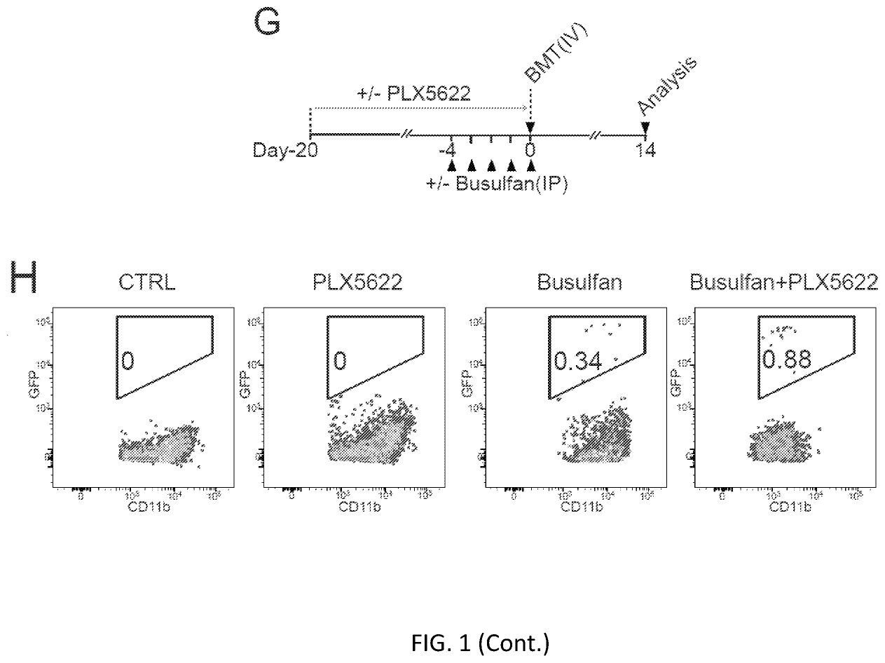

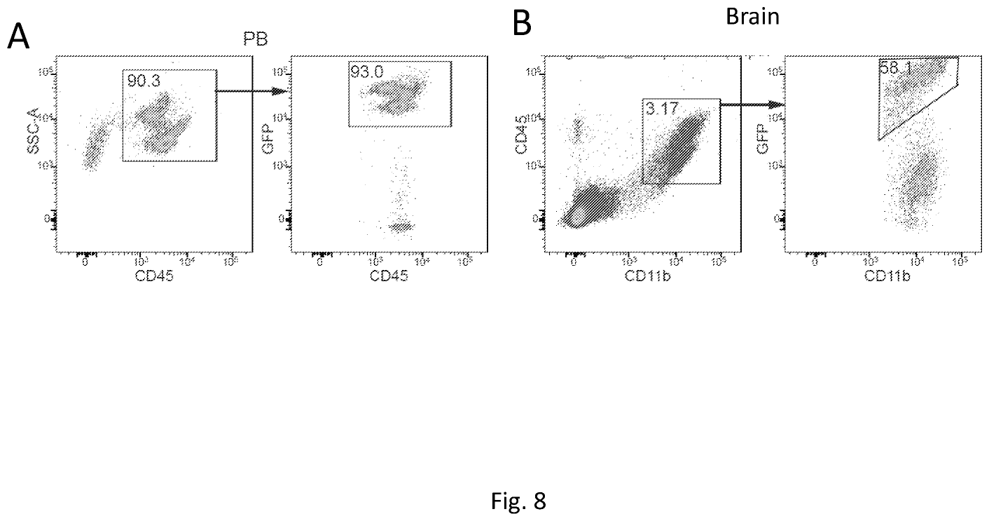

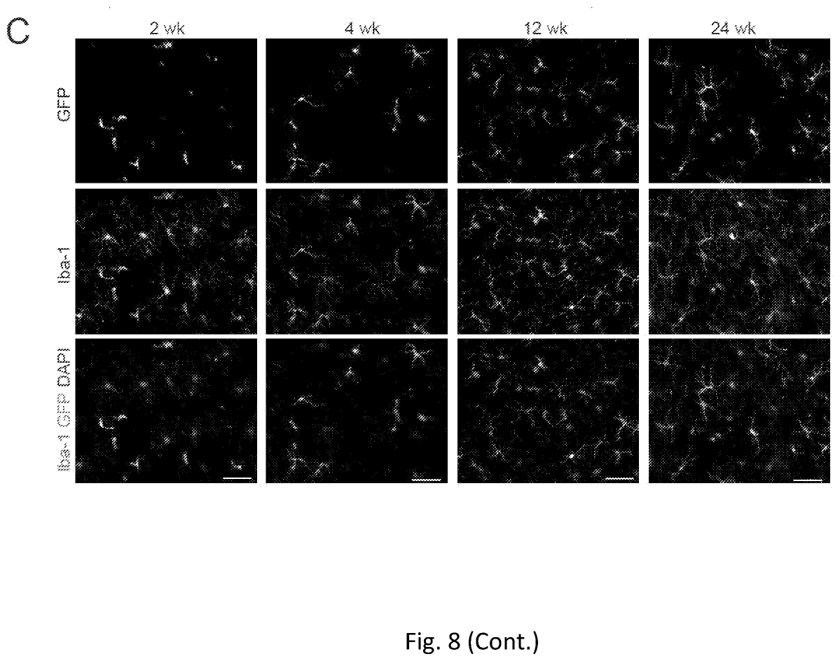

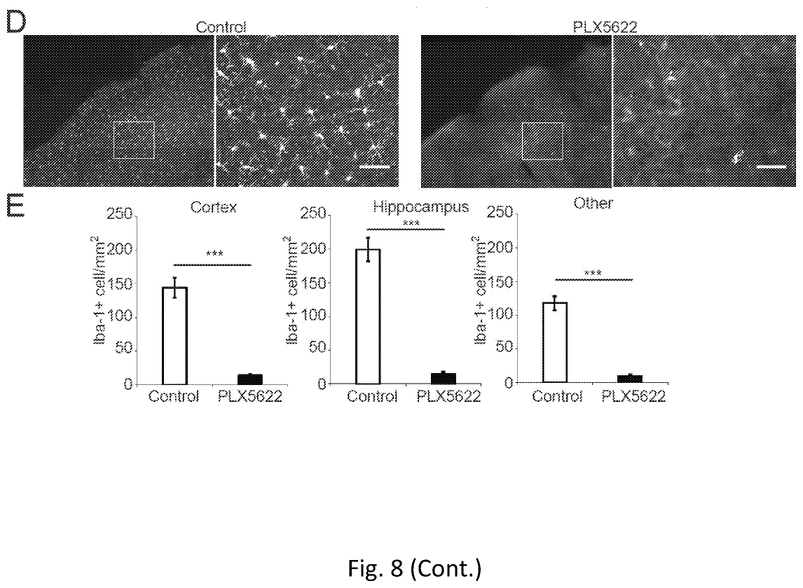

The reported efficiencies of microglia replacement following HSCT in mice vary greatly in the literature. In order to improve CDMC engraftment into the brain we sought to better characterize the process. We injected the DNA-alkylating chemotherapeutic agent busulfan every day for 5 consecutive days to WT recipient mice followed by intravenous (IV) injection of freshly isolated bone marrow cells from a human ubiquitin C promoter-EGFP transgenic mice (FIG. 1A). Blood chimerism was tested by venipuncture followed by flow cytometry. Brain chimerism was determined by sacrificing the mice, dissociating one hemisphere for flow cytometry analysis and fixing the other hemisphere for immunofluorescence analysis (FIG. 8A-C).

To evaluate chimerism in all myeloid cells of the brain, both CD45^{hi}CD11b⁺ (macrophage gate) and CD45^{int/lo}CD11b⁺ cells (microglia gate) were analyzed (FIG. 8B). Surprisingly, the engraftment of GFP⁺ cells into the brain was much slower than GFP chimerism in the peripheral blood; while the blood contained on average about 40% GFP⁺ cells as early as 2 weeks after transplantation, it took 12 weeks for a notable GFP chimerism among the myeloid cells of the brain to develop (FIG. 1B, C). As expected, these GFP⁺ cells in the brain adopted a microglia-like morphology (FIG. 8C). Of note, the degree of chimerism was highly variable between mice ranging from 7.09-73.4%. Unlike in the blood, the degree of chimerism plateaued in the brain and did not significantly increase further even 6 months after transplantation. We could also confirm previous reports that an about 25% higher dose of busulfan is needed for brain engraftment than for replacement of the bone marrow. Despite this maximally tolerable dose and extended incorporation times the degree of microglia replacement remained low and highly variable compared to high and consistent chimerism in the peripheral blood.

The Microglia Niches Need to be Vacant to Allow Bone Marrow Cells to Engraft.

The effects of chemotherapy on microglia can be complex. There is a reduction of endogenous microglia by brain-permeable cytostatic treatments (like busulfan) but not a brain-impermeable cytostatic drug even though the majority of microglia in the normal mouse brain are not cycling. To directly test whether microglia depletion facilitated CDMC engraftment we injected GFP-labeled bone marrow cells directly into the brain of WT mice with or without a 21-day pre-treatment of the CSF1R inhibitor PLX5622. Pharmacological CSF1R inhibition leads to an efficient elimination of endogenous microglia throughout the brain (FIG. 8D, E). Strikingly, while bone marrow cells injected into the control brain remained unchanged, the cells injected into PLX5622-treated mice adopted microglia-like morphologies, expressed the microglial marker Iba-1, were more numerous, and dispersed wider into the brain parenchyma (FIG. 1D-F). Thus, the elimination of endogenous microglia was critically required for bone marrow cells to engraft in the brain and differentiate into microglia-like cells.

Microglia Depletion Before HSCT is Insufficient to Enhance Microglia Replacement.

Given the importance to empty microglial niches for microglia replacement we next tested whether a PLX5622 treatment before HSCT enhanced incorporation of CDMCs into the brain, but found only a marginal increase in the fraction of GFP⁺ cells among brain myeloid cells in the busulfan+PLX5622 group (FIG. 1G, H).

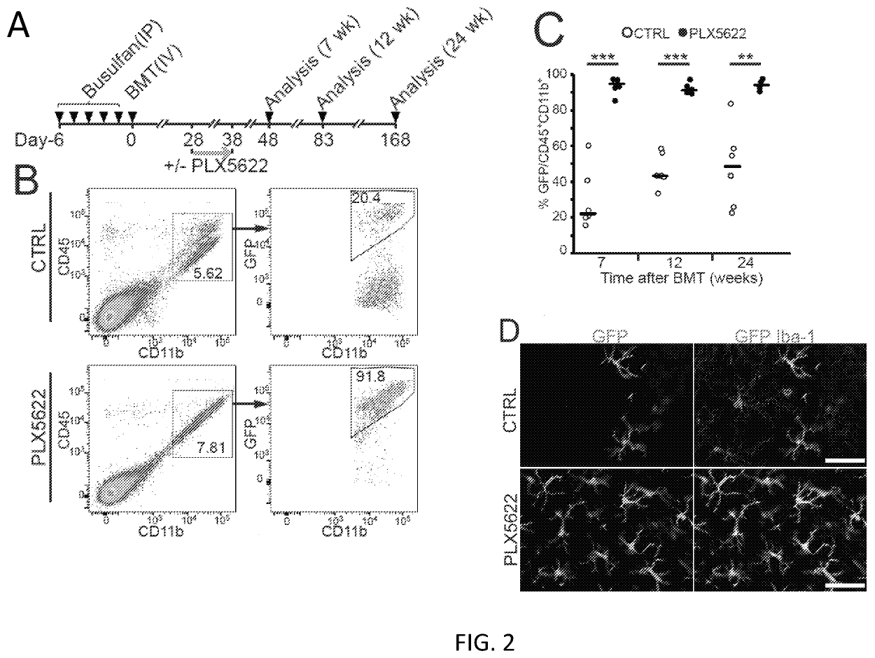

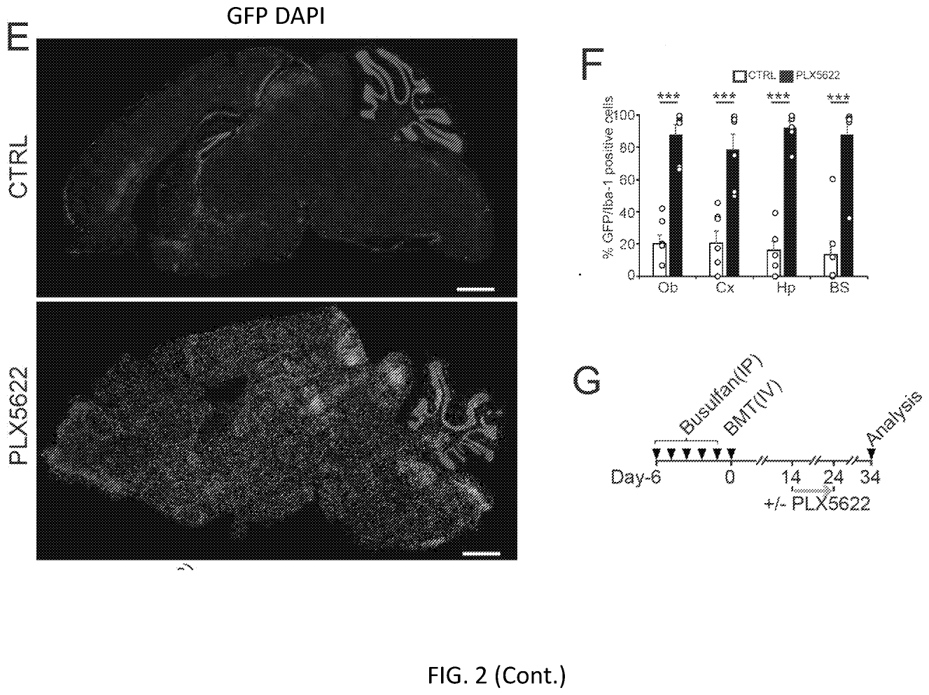

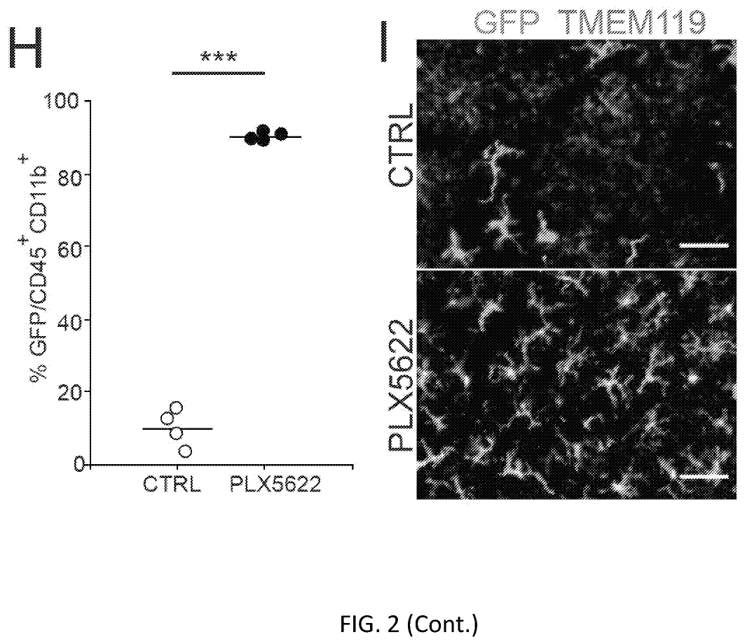

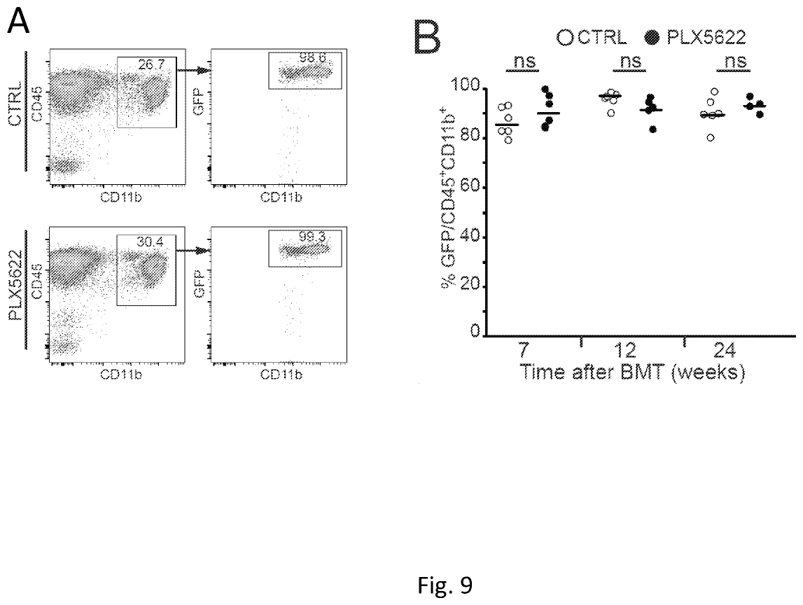

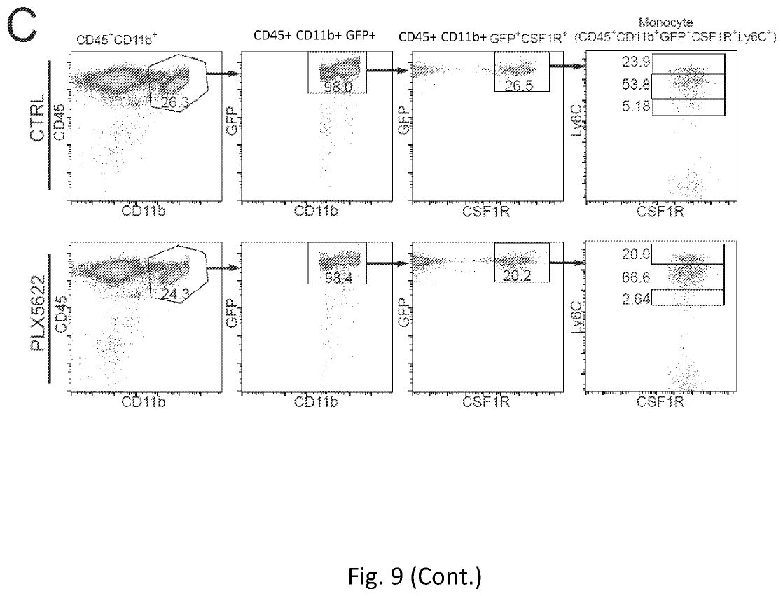

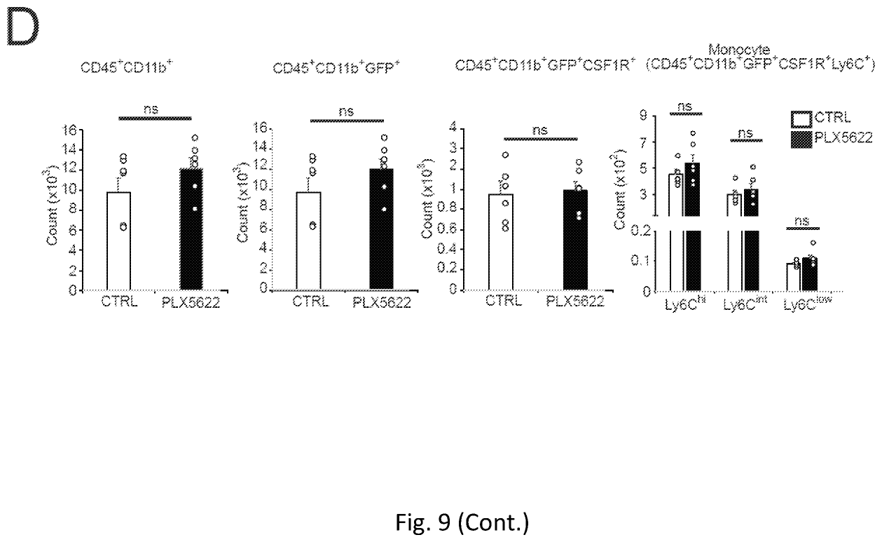

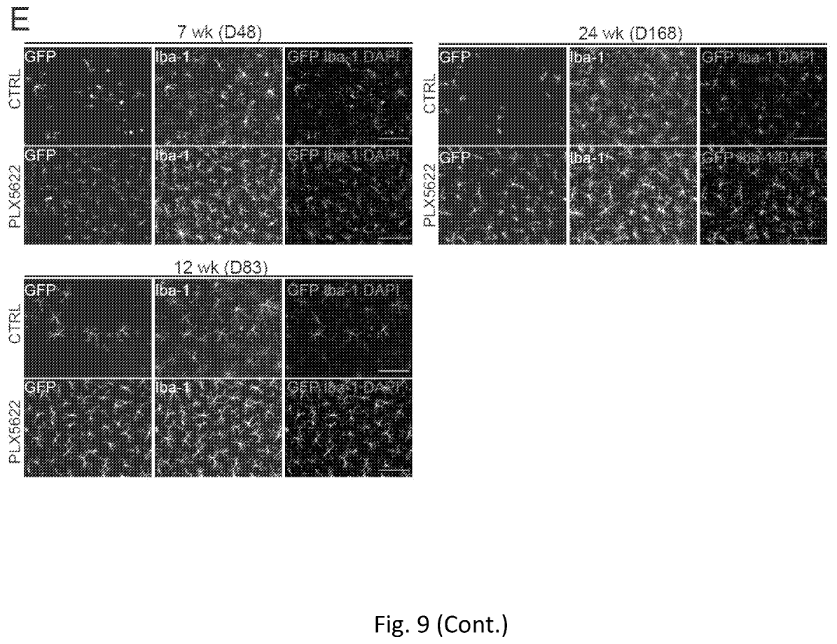

Microglia depletion after HSCT results in near complete replacement of microglia with bone marrow-derived cells throughout the entire brain. We next tested whether CSF1R inhibition after HSCT enhanced microglia replacement. We therefore performed conventional HSCT with GFP-labeled fresh bone marrow cells. After 28 days, we treated the experimental group with PLX5622 for 10 days and the control group received no drug (FIG. 2A). After another 10 days (7 weeks post transplant) we analyzed the brains and consistently found over 90% of all brain myeloid cells to be GFP⁺ by flow cytometry (FIG. 2B, C). Histological analysis revealed that the vast majority of GFP⁺ cells had adopted a microglia-like morphology and stained for Iba-1 in multiple brain regions (FIG. 2D, F). The transplanted CDMCs had homogeneously populated the entire brain (FIG. 2E). Importantly, this high chimerism was stable and CDMCs remained incorporated for at least 6 months after transplantation (FIG. 2C, 9E). Despite the pronounced effects of CSF1R inhibition on the brain, the composition of peripheral blood cell populations was largely unaffected (FIG. 9A-D).

We next tested the PLX5622 treatment sooner after HSCT and found that treatment between days 14 and 24 after HSCT resulted in comparably robust and efficient engraftment of CDMCs into the brain (FIG. 2G, H). Notably, PLX5622 treatment following HSCT induced expression of the microglia-specific marker TMEM119 in CDMCs (FIG. 21). TMEM119 protein is not expressed in peripheral macrophages and CDMCs after conventional HSCT without PLX5622 treatment, suggesting that repopulated CDMCs after PLX5622 treatment acquire a more microglia-like phenotype (FIG. 21).

CDMCs Incorporated into the Brain are Slightly More Activated than Resting Microglia.

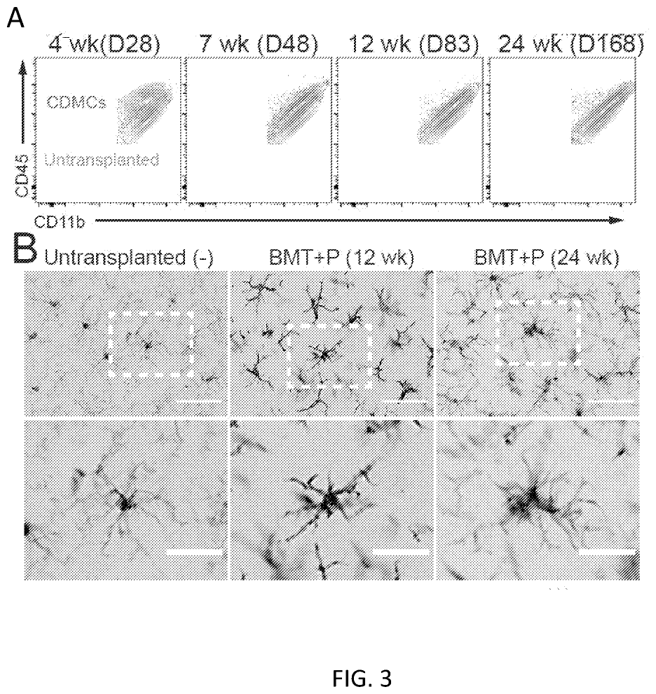

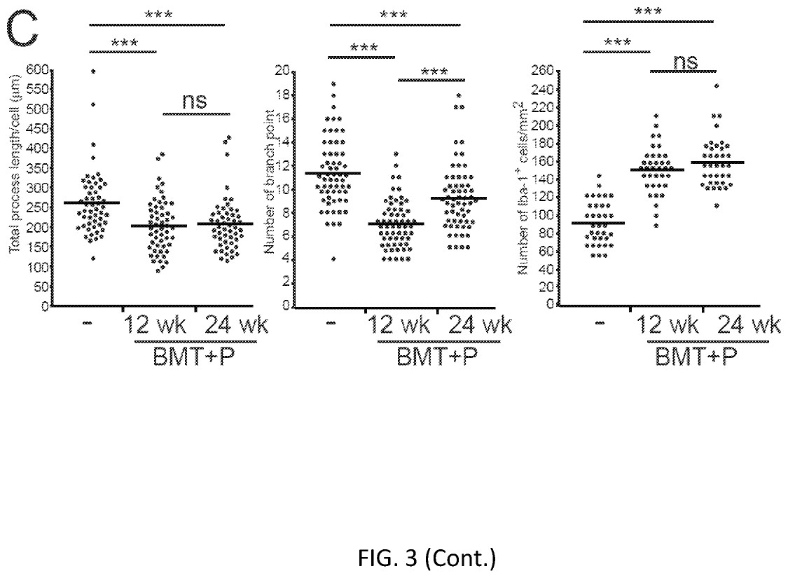

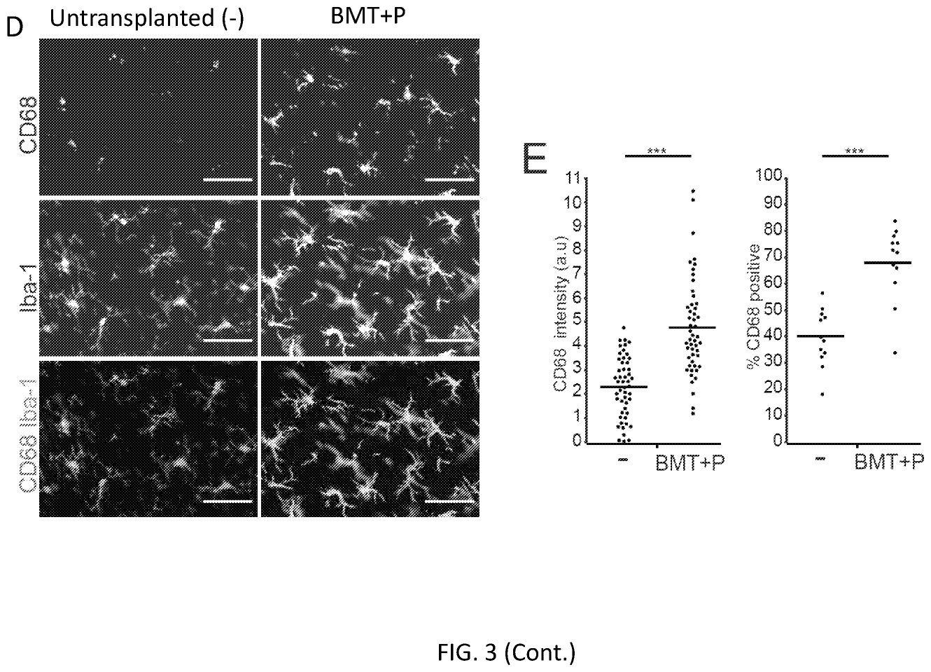

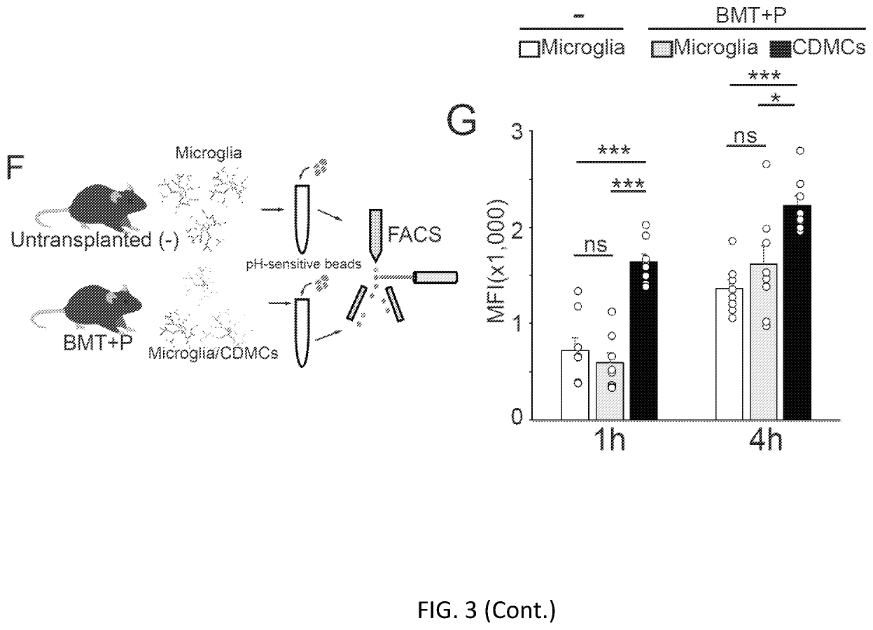

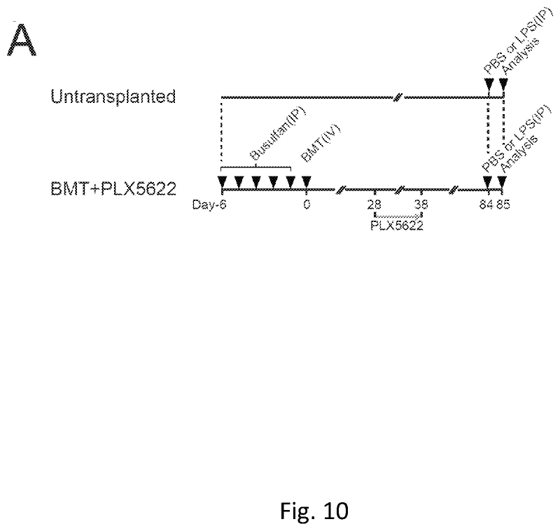

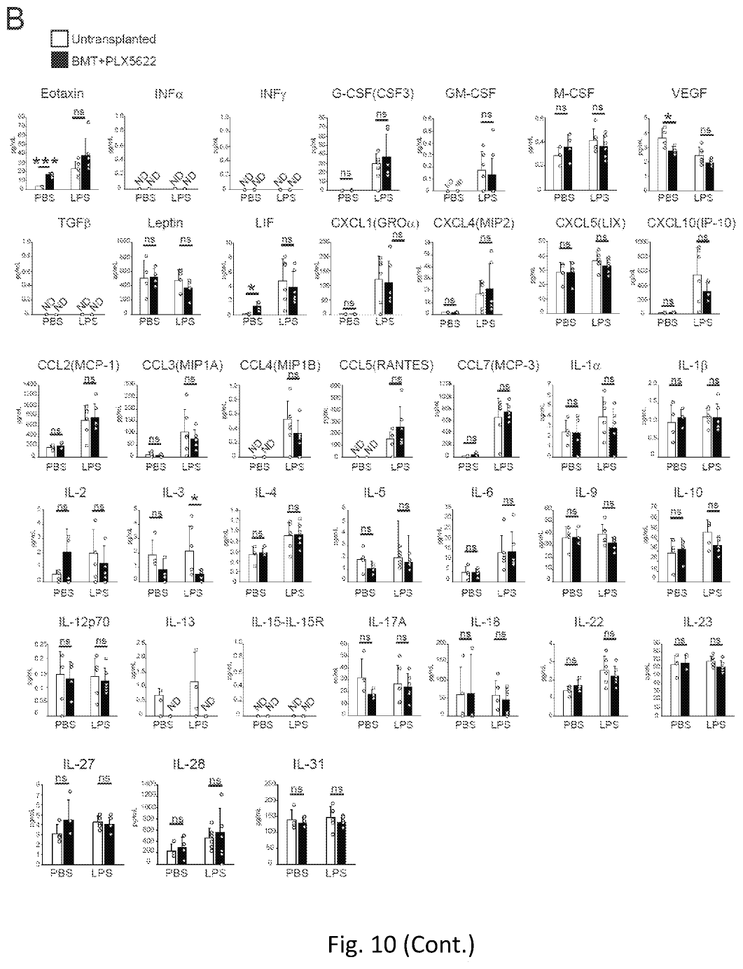

The expression of TMEM119 in CDMCs prompted us to better characterize the CDMCs incorporated into the brain following our high-efficiency HSCT and PLX5622 treatment and recovery (HSCT+P) method. First, we noticed that CDMCs had slightly higher levels of CD45 and slightly lower levels of CD11b than microglia by flow cytometry analysis (FIG. 3A). Remarkably, CDMCs following PLX5622 recovery (from 7 weeks post transplantation and onward) were more similar to microglia than following conventional HSCT (4 weeks post transplantation before PLX5622 treatment) based on these 2 markers and became even more similar after extended incorporation times (FIG. 3A). While CDMCs after HSCT+P rapidly adopted a microglial morphology, their processes were thicker, shorter, and less branched (FIG. 3B-C), which are typical characteristics of activated microglia. The number of branched processes increased over time but was still significantly lower than microglia 24 weeks after transplantation. Another notable difference was that the density of CDMCs was stably higher than endogenous microglia (FIG. 3C). In line with their slightly more activated morphology, CDMCs after HSCT+P expressed higher levels of CD68 (FIG. 3D, E) and showed an increased phagocytosis activity based on an in vitro bead phagocytosis assay (FIG. 3F, G). To further characterize CDMC function, we systemically injected PBS or LPS into mice with or without HSCT+P and measured a panel of cytokines in the brain by a multiplex bead-based assay (FIG. 10A). Without LPS stimulation, 5 out of 38 cytokines (Eotaxin, VEGF, LIF, IL3, IL13) examined were differentially produced in the brain of CDMC-containing and intact mice; however, the relative response of all cytokines in the brain to LPS stimulation was similar between the two groups (FIG. 10B).

Engrafted CDMCs after Repopulation are More Similar to Microglia than CDMCs Incorporated after Conventional HSCT.

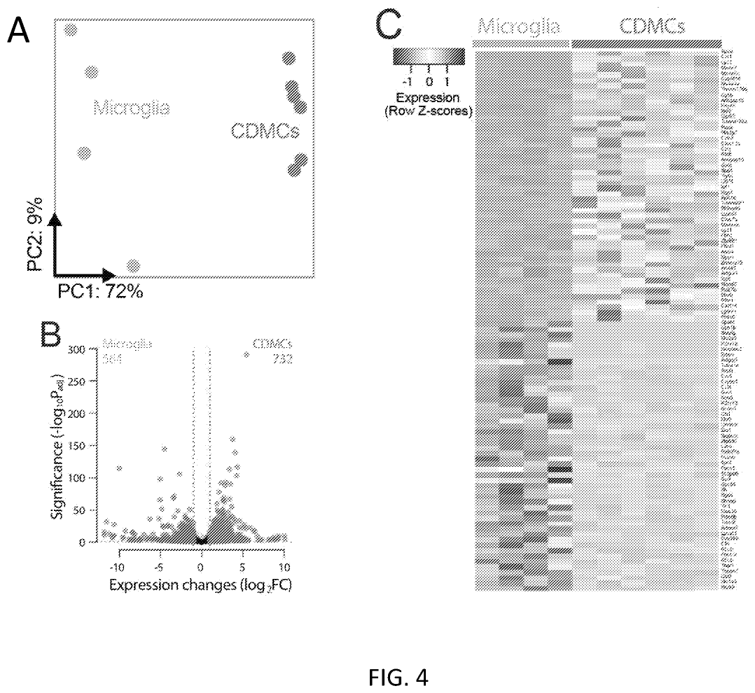

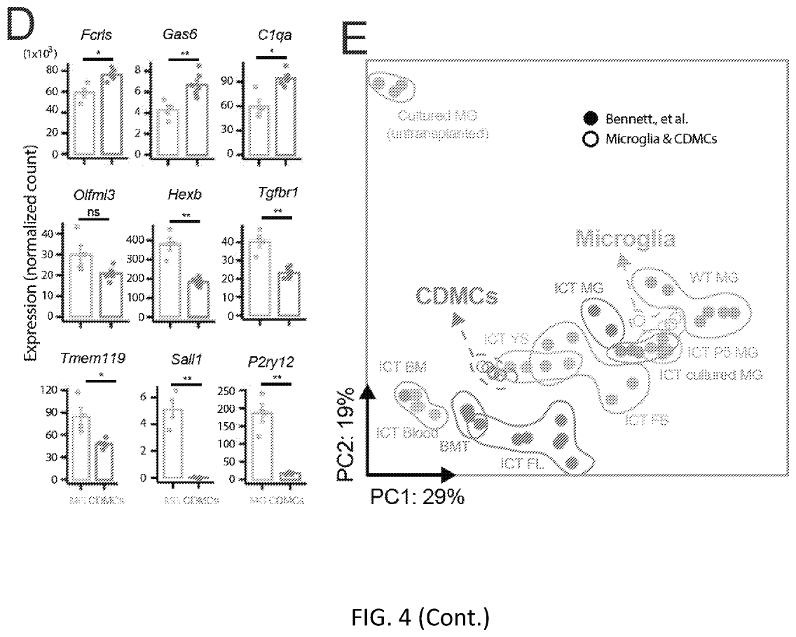

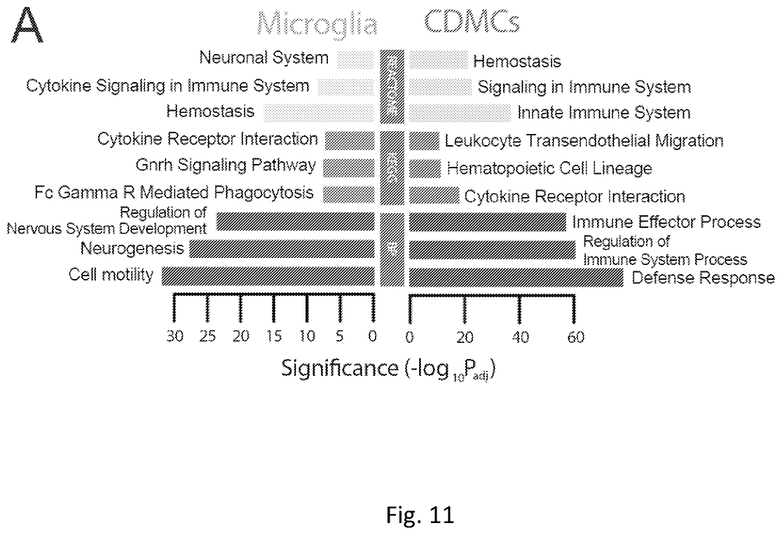

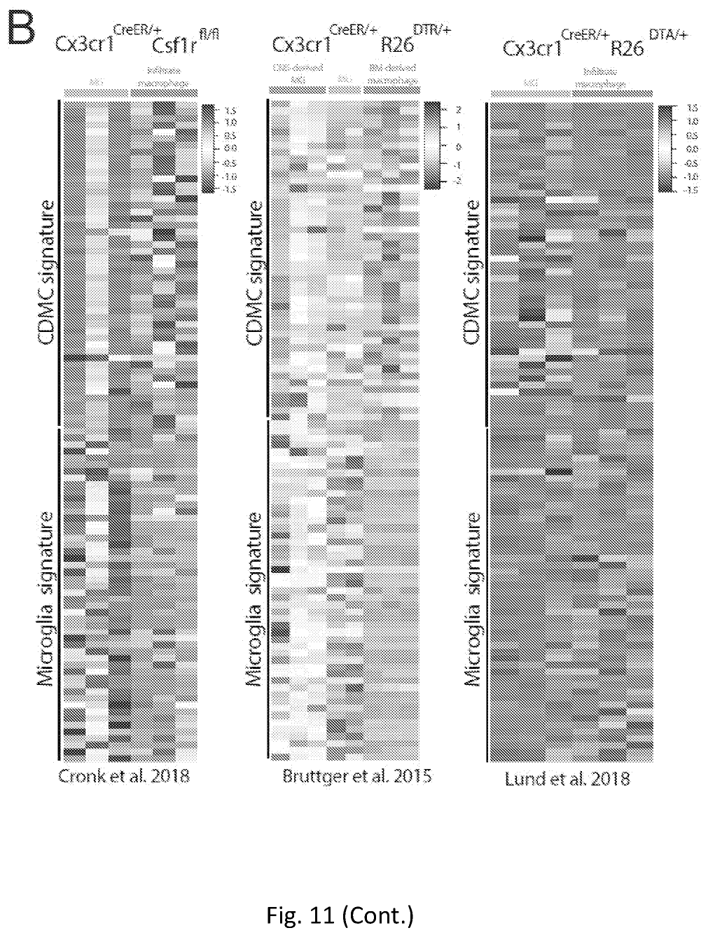

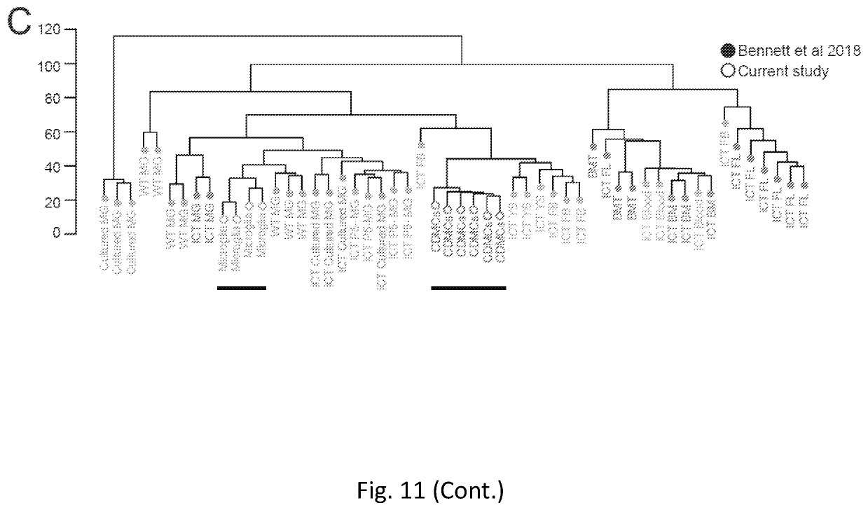

We next examined the genome-wide expression profile of CDMCs replacing virtually all microglia following our HSCT+P protocol by RNA-sequencing (RNA-seq). As expected, the CDMC samples isolated from the brain of mice that had received HSCT+P (12 weeks post transplant) and microglia samples isolated from the brain of age-matched untransplanted mice clustered as 2 distinct groups in the principal component analysis (PCA) (FIG. 4A). There were 1296 genes differentially expressed between microglia (564) and CDMCs (732) (FIG. 4B; Table 1). Gene Ontology analysis showed that CDMC-enriched GO terms were mainly associated with immune response whereas microglia-enriched GO terms were associated with neuronal processes in addition to immune response (FIG. 11A). The top 50 microglia-enriched genes contained previously identified microglia-specific genes such as P2ry12, Ctsl, Tgfa, Sall1 and Sall3 (Butovsky et al., 2014; Cronk et al., 2018) and there was a good overlap between the top 50 microglia and CDMC-enriched genes in our dataset and three other microglia replacement strategies using genetic means to deplete microglia (FIG. 4C; 11B, Table 1). As expected, some critical microglial genes showed similar or higher expression in our CDMCs (Fcrls, Gas6, C1qa), others were expressed but to lower levels (Olfml3, Hexb, Tgfbr1, and Tmem119), and some lowly to not expressed (Sall1, P2ry12) (FIG. 4D, (FIG. 2I).

To more comprehensively characterize our CDMCs, we compared our dataset to published gene expression profiles of microglia-like cells derived from different hematopoietic cell populations. A previous study showed that multiple hematopoietic cell populations acquire microglia-like signatures in the brain following systemic or intracranial injection into microglia-deficient Csf1r−/− mice but only those of yolk-sac origin completely attained microglia identity. Remarkably, our microglia samples (green open circles) clustered very closely to the published microglia samples in the PCA plot (FIG. 4E) and using unbiased hierarchical clustering (FIG. 11C). More intriguingly, as shown in the PCA plot in FIG. 4E, the CDMCs in our HSCT+P model (red open circles) were modestly distinct from bone marrow-derived cells either directly injected into the brain (ICT-BM and ICT-Blood, green filled circles) or systemically injected to the mice (HSCT, blue filled circles). While the difference was only modest, the location of the cluster of our CDMCs was on the axis between the conventionally engrafted bone marrow cells and endogenous microglia and overlapping with the clusters of yolk-sac-derived and fetal brain-derived microglia-like cells (FIG. 4E, 11C). Our findings suggest that our CDMCs are distinct from conventionally repopulated CDMCs and somewhat more similar to endogenous microglia, most closely resembling yolk sac-derived cells. These results are compatible with our previous observation that protein levels of CD45, CD11b, and TMEM119 more similar to microglia than conventional bone marrow cells incorporated into the brain.

The high CDMC brain chimerism is best explained by proliferative advantage of few CDMCs during the PLX5622 withdrawal period rather than continued influx from the circulation. We next wanted to know the origin of the homogeneously engrafted CDMCs after the PLX5622 treatment. Given that the donor chimerism in the blood before and after PLX5622 treatment was close to 100% (FIG. 1B), we wanted to investigate whether the newly repopulated GFP⁺ cells resulted from an expansion of previously engrafted brain CDMCs or whether they were continuously recruited from the circulation.

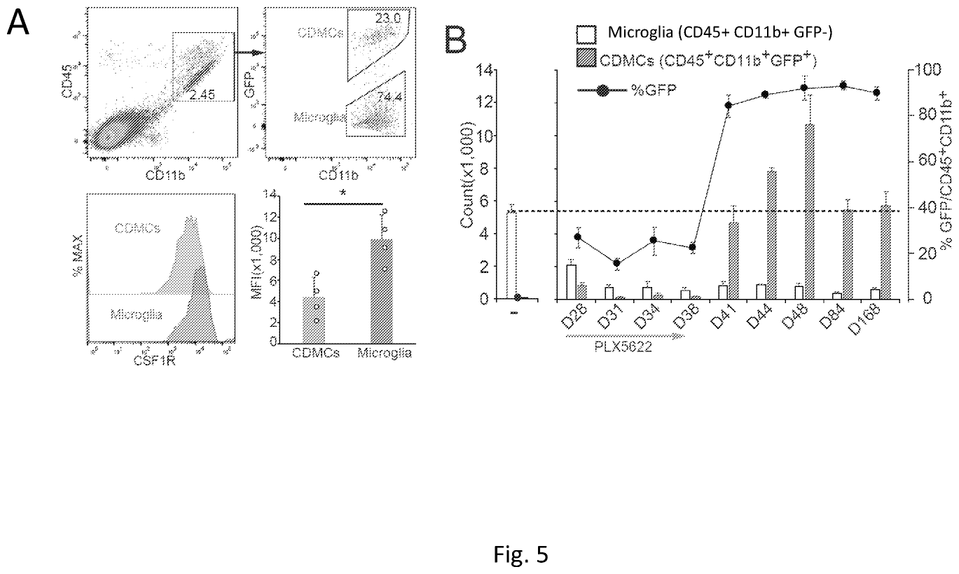

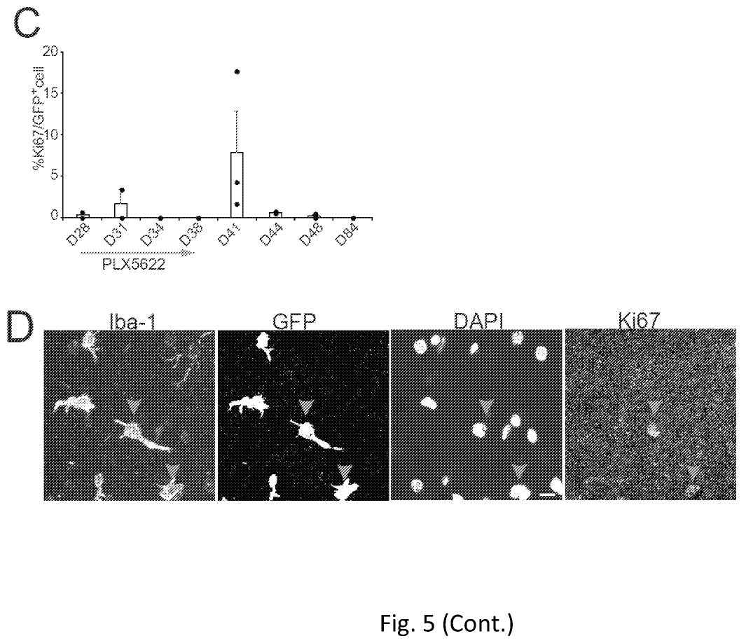

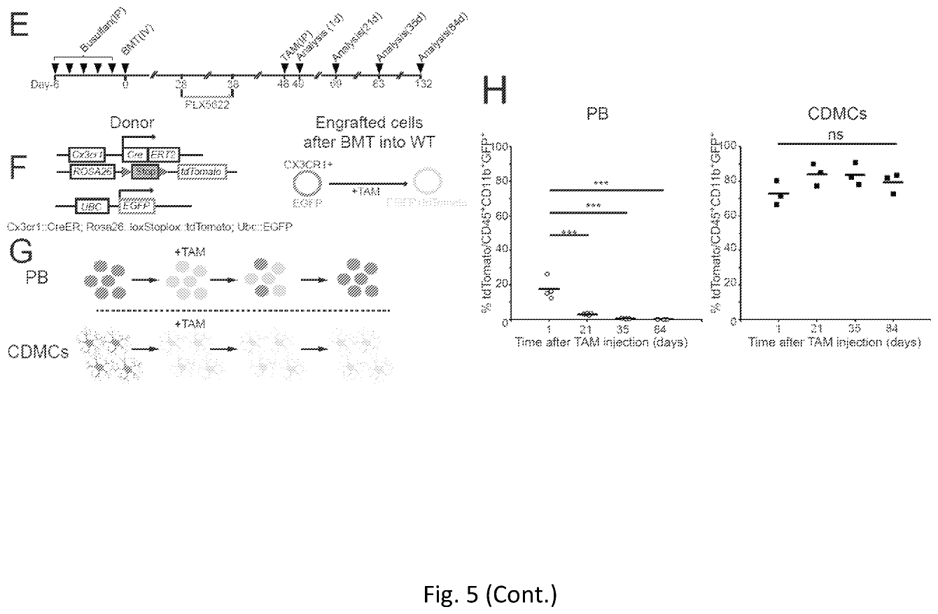

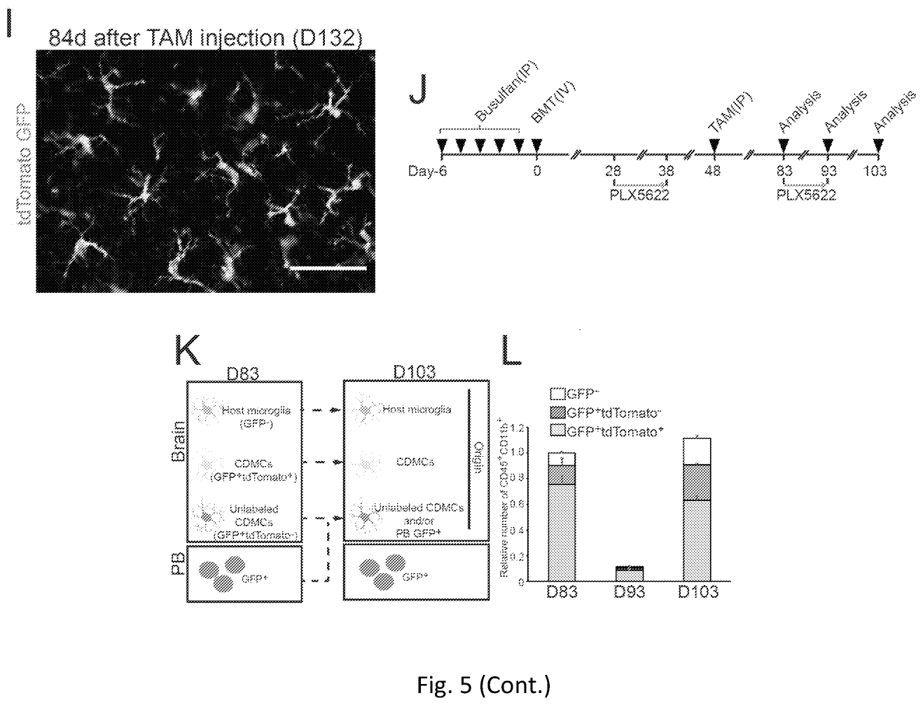

First, we hypothesized that CDMCs might be less sensitive to CSF1R inhibition which would lead to a selective elimination of microglia and growth advantage of CDMCs. Indeed, FACS analysis showed that CDMCs expressed lower levels of CSF1R (FIG. 5A). However, when we quantified CDMCs (GFP+) and microglia (GFP−) during PLX5622 treatment by flow cytometry, both cell populations were diminished to similar degrees (blue arrow in FIG. 5B). It was only after removal of CSF1R inhibition when CDMCs were primarily detected (FIG. 5B, after day 41).

We next quantified the fraction of proliferative CDMCs during and after PLX5622 treatment by staining brain sections with Ki67 and Iba-1. The GFP+ CDMCs were essentially negative for Ki67 at all time points analyzed except on D41 the first time point after PLX5622 withdrawal (FIG. 5B, C). Thus, at least in part the CDMC overrepresentation can be explained by proliferation of brain-resident CDMCs that survived the PLX5622 treatment.

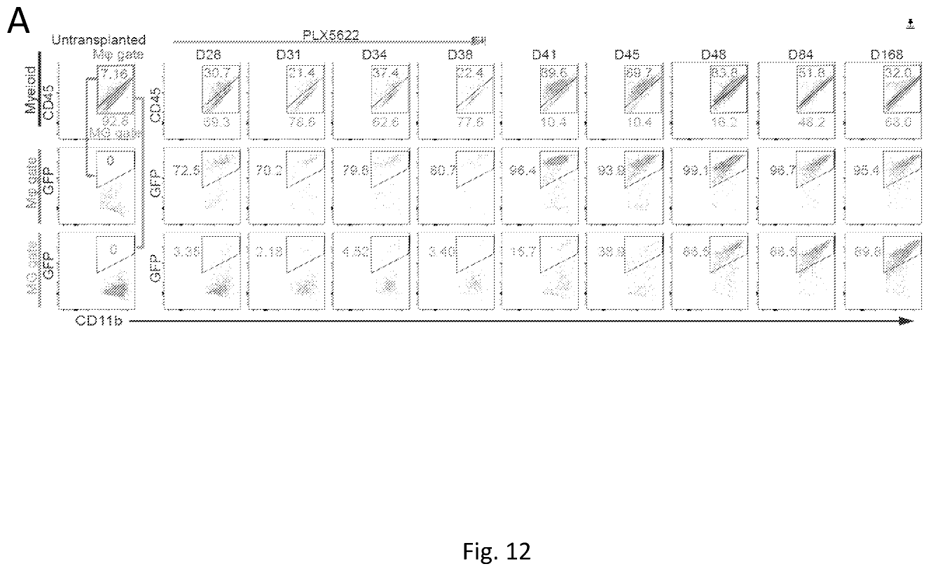

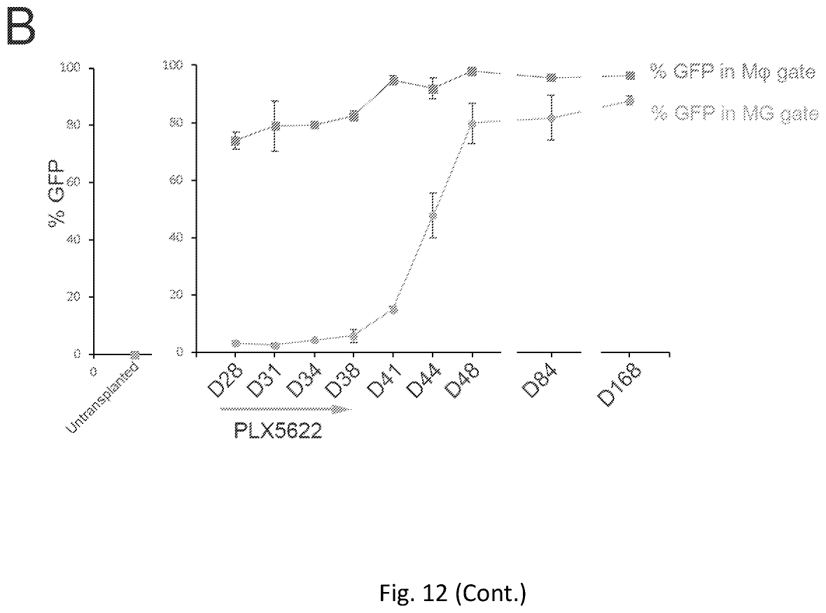

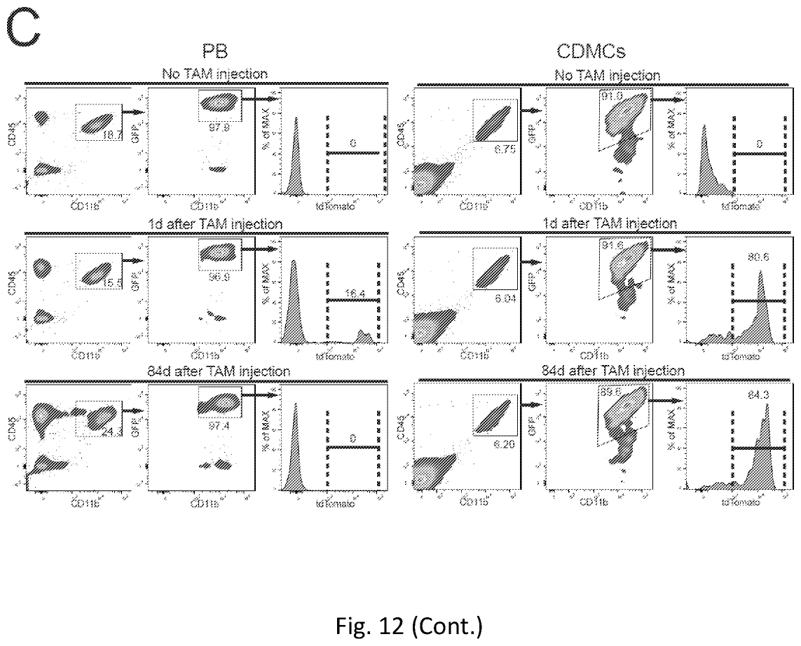

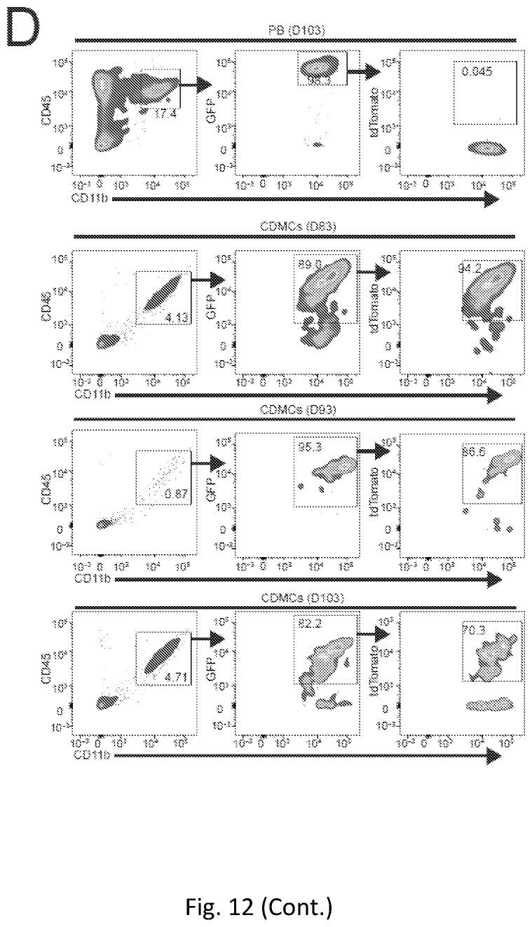

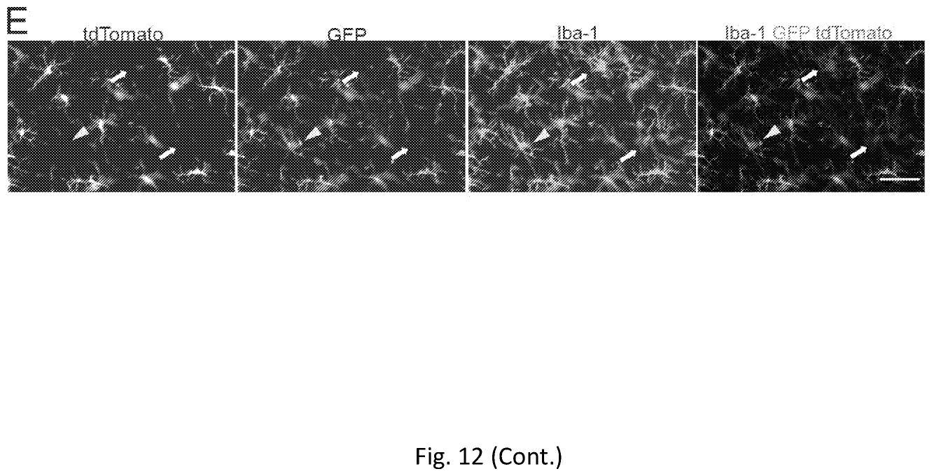

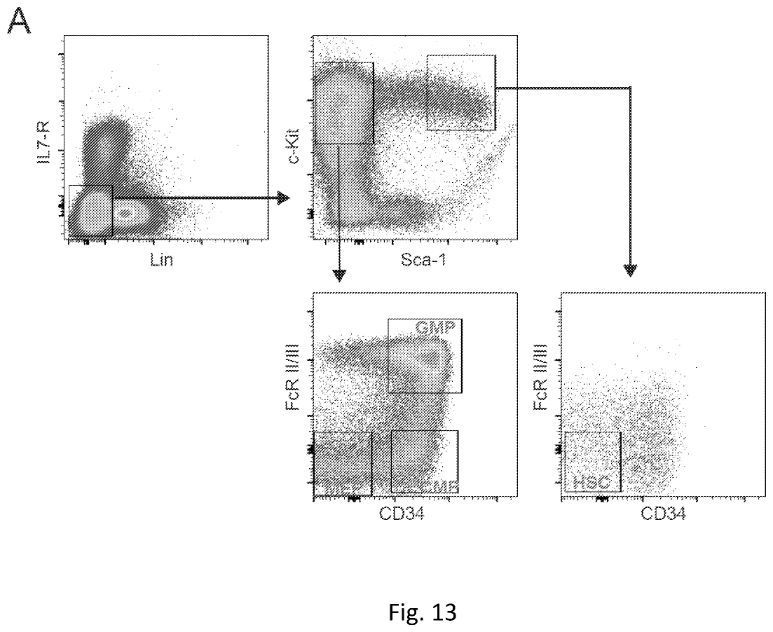

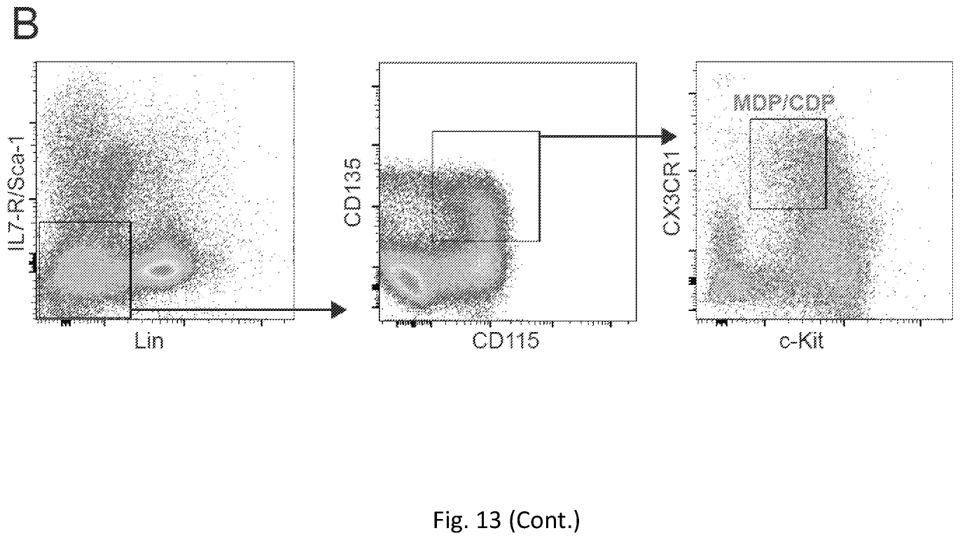

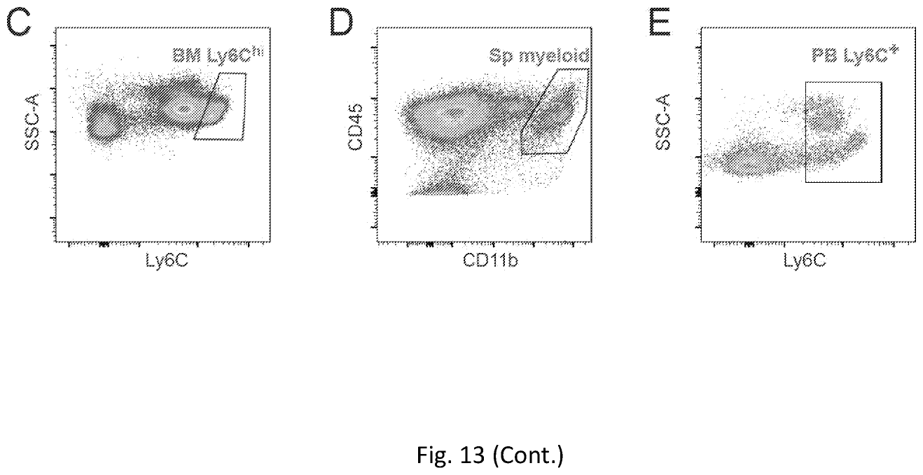

Careful flow cytometric analysis of CD45 and CD11b expression showed that CDMCs primarily populated the CD45^{hi}CD11b⁺ “macrophage” gate before and during PLX5622 treatment but gradually migrated into the CD45^{lo/int}CD11b⁺ “microglia” gate immediately after PLX5622 withdrawal (FIG. 12A, B). This result further indicates that the CDMCs generated after PLX5622 removal are derived from brain-resident cells rather than infiltrating macrophages.

To more directly investigate a potential contribution of newly populated GFP+ cells from the circulation following PLX5622 treatment, we explored ways to label the circulating cells with a different fluorescent color than the CDMCs. To this end, we used Cx3cr1::CreER; Rosa26::loxStoplox::tdTomato; Ubc::EGFP mice as donors for HSCT (FIG. 5E, F). In these mice, all cells are constitutively GFP+ and all Cx3cr1+ myeloid cells in the blood and brain will become tdTomato+ upon tamoxifen exposure (FIG. 5F). Because microglia and CDMCs in the brain are long lived, they will remain tdTomato+ for a long time. Peripheral blood cells, however, would be expected to be replaced by tdTomato-, GFP⁺ cells over time because of the rapid cell turn over of monocytes (FIG. 5G). Indeed, about 80% of CDMCs were stably tdTomato+ at least up to 84 days after tamoxifen, whereas the percentage of tdTomato+ cells rapidly decreased in the blood and was not detectable anymore by day 35 (FIG. 5H, I, 12C), establishing a mouse model that contained exclusively GFP+dtTomato− blood cells and primarily GFP+tdTomato+CDMCs in the brain.

We next reconstituted microglia and the bone marrow of WT mice with cells from these triple transgenic mice using our HSCT+P method and injected tamoxifen on day 48, a time point of complete brain microglia replacement. We then waited for 35 days to allow cell turnover in the blood before the second round of 10-day PLX5622 treatment (FIG. 5J). At this time point, most CDMCs in the brain will be GFP+tdTomato+ (only few CDMCs will be GFP+tdTomato− due to inefficiency of tamoxifen) and all peripheral blood cells GFP+tdTomato− (FIG. 5K). We sacrificed mice before PLX5622 treatment (d83), directly after the 10-day treatment period (d93), and 10 days after the drug withdrawal (d103). Flow cytometry analysis showed that as observed before (FIG. 5B) both microglia (CD45+CD11b+GFP−) and CDMCs (CD45+CD11b+GFP+tdTomato+/−) were depleted by CSF1R inhibition (FIG. 12D, 5L). Importantly, after the drug withdrawal and complete regeneration of CDMCs, the largest fraction of CDMCs were GFP+tdTomato+ (FIG. 5L, 12E), clearly demonstrating that at least the majority of CDMCs found after the drug recovery period are in fact derived from pre-existing, brain-resident CDMCs.

Hematopoietic Stem Cells (HSCs) are the Most Efficient Cells Engrafting into the Brain.

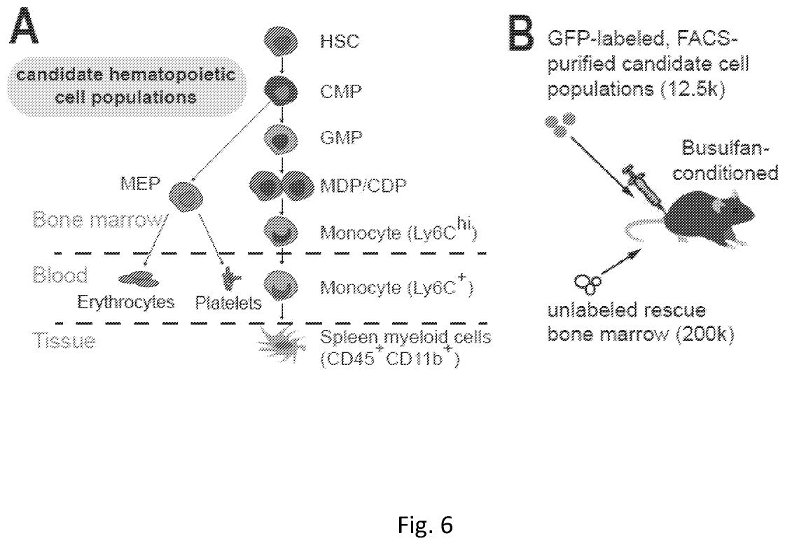

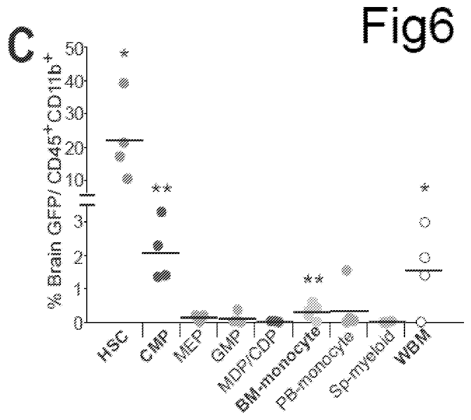

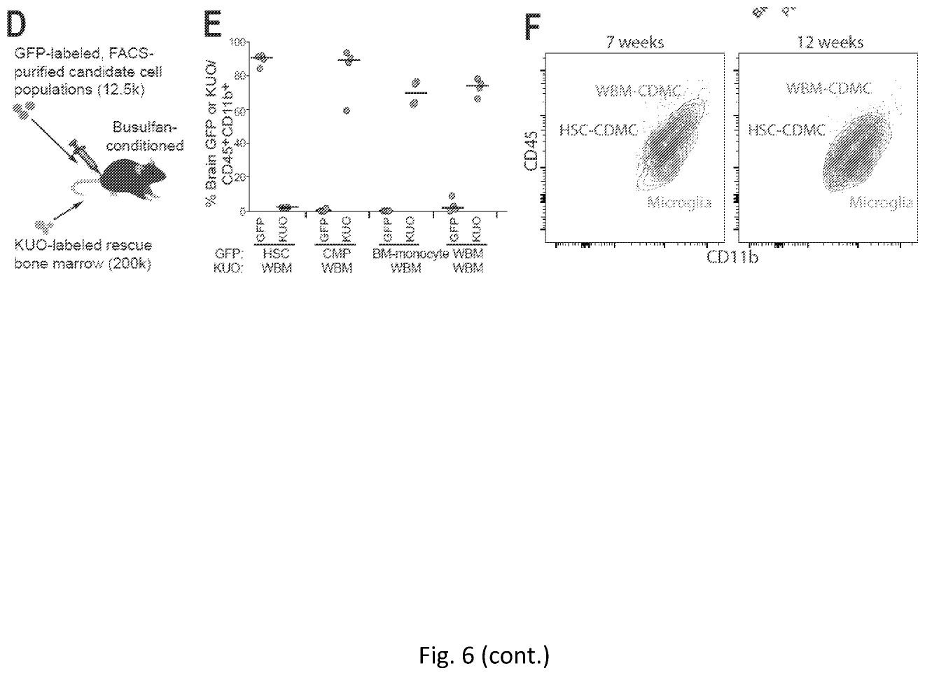

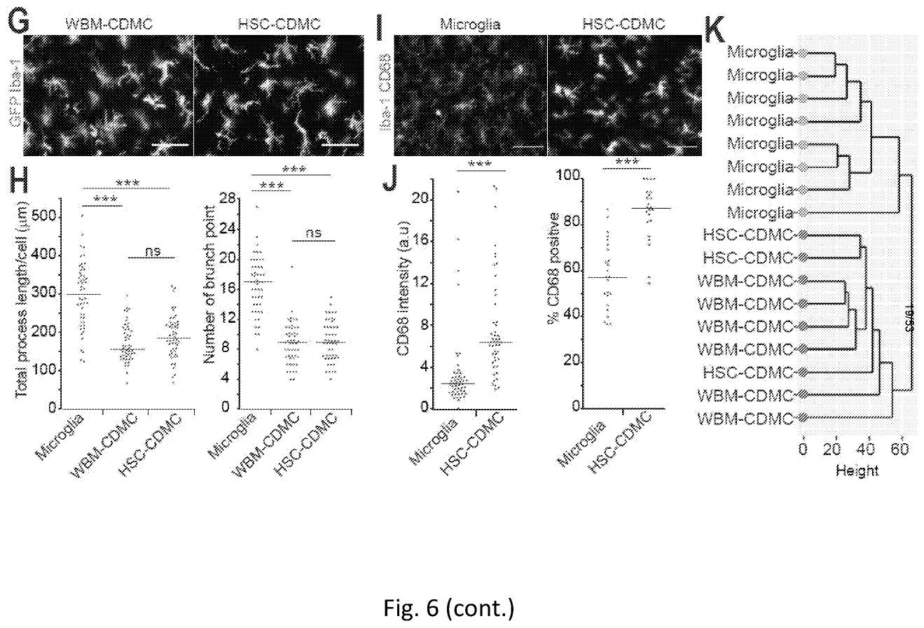

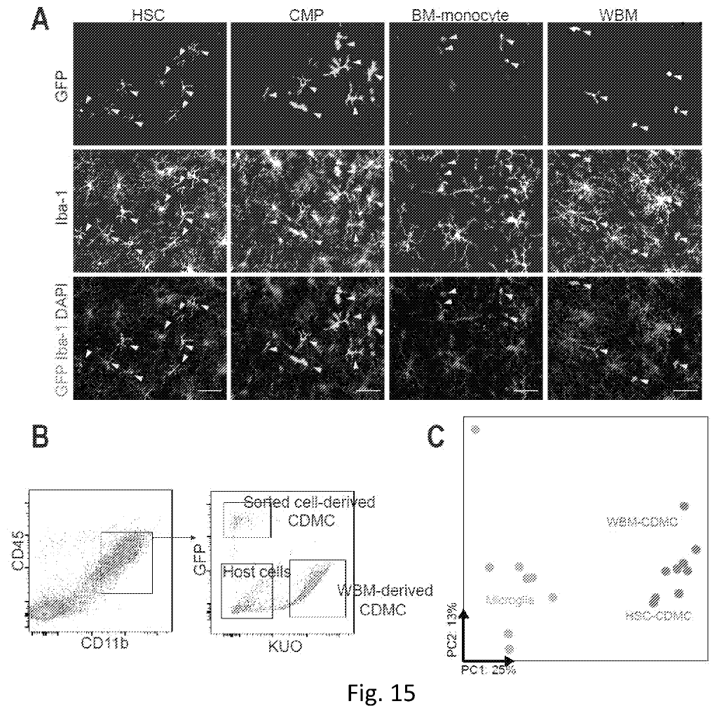

Hematopoietic stem cells (HSCs) are equivalent to whole bone marrow cells in their ability to engraft as myeloid cells into the brain. Whole bone marrow (WBM) is heterogeneous and ill-defined. We therefore sought to evaluate which specific hematopoietic cell population had the highest capacity of generating CDMC engraftment. To this end, we purified and injected 8 distinct hematopoietic cell types and WBM as positive control into busulfan-conditioned mice (FIGS. 6A and B). Unlabeled helper WBM cells were injected a day later to rescue from lethal busulfan conditioning (FIG. 6B). Four weeks later, we detected statistically significant GFP myeloid chimerism in the brain following transplantation of HSCs, common myeloid progenitors (CMPs), bone marrow monocytes, and WBM (FIG. 6C). Grafted HSCs resulted in by far the highest myeloid chimerism in the brain ranging from 10-40%. Moreover, HSC-derived CDMCs displayed already microglia-like ramified morphology unlike all other populations (FIG. 15A).

We next sought to determine whether any of these 4 significant populations could give rise to high chimerism following PLX5622 treatment as seen with WBM. We therefore repeated the experiment with the 4 populations, this time using Kusabira orange (KUO)-labeled helper WBM (33) (FIG. 6D). Ten days after PLX5622 withdrawal, the HSC-derived CDMCs had almost completely outcompeted the helper WBM, unlike the other 3 populations (FIG. 6E). The morphology of incorporated HSC-derived CDMCs are most similar to WBM cells (FIG. 15A). These results show that the brain incorporation activity is highly enriched in HSCs.

Remarkably, HSC-derived CDMCs and WBM-derived CDMCs had similar characteristics. The relative expression of CD45 and CD11b over time as measured by FACS was almost exactly the same in the two populations (FIG. 6F). Both populations had a similar morphometric profile (FIGS. 6G and H). Just like WBM-CDMCs, HSC-derived CDMCs expressed higher levels of CD68 than microglia (FIGS. 6 I and J). Finally, RNA-seq analysis showed that HSC- and WBM-CDMCs clustered together in unsupervised hierarchical clustering and the PCA, respectively (FIG. 6K and FIG. 15C). These results demonstrate that microglia can be efficiently replaced after transplantation with purified HSCs and HSC-derived CDMCs are morphologically and transcriptionally comparable to WBM-derived CDMCs.

The Aged Brain Environment Efficiently Makes Newly Populated CDMCs “Old”.

We next sought for a potential application of our efficient microglia replacement strategy to answer important biological questions. During aging, microglia undergo phenotypic and functional changes that are likely contributing to the age-related decline in brain function. However, it is not completely understood whether these changes are resulting from intrinsic microglial aging or caused by extrinsic factors.

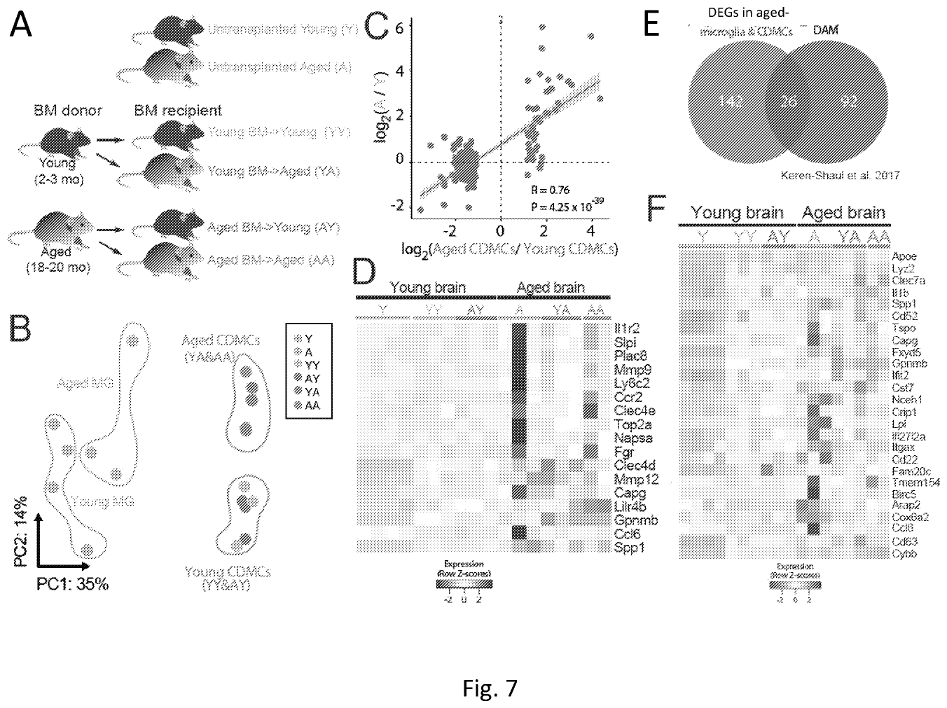

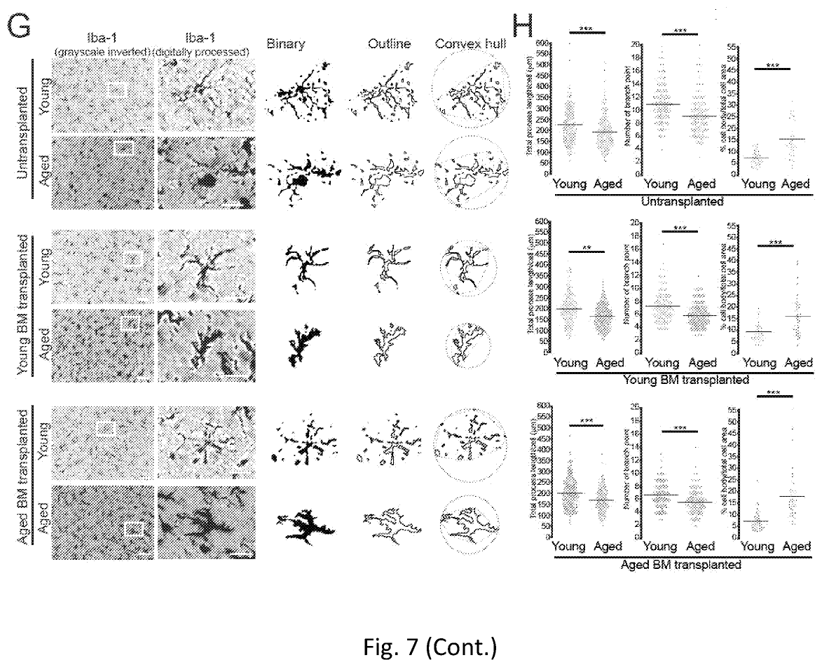

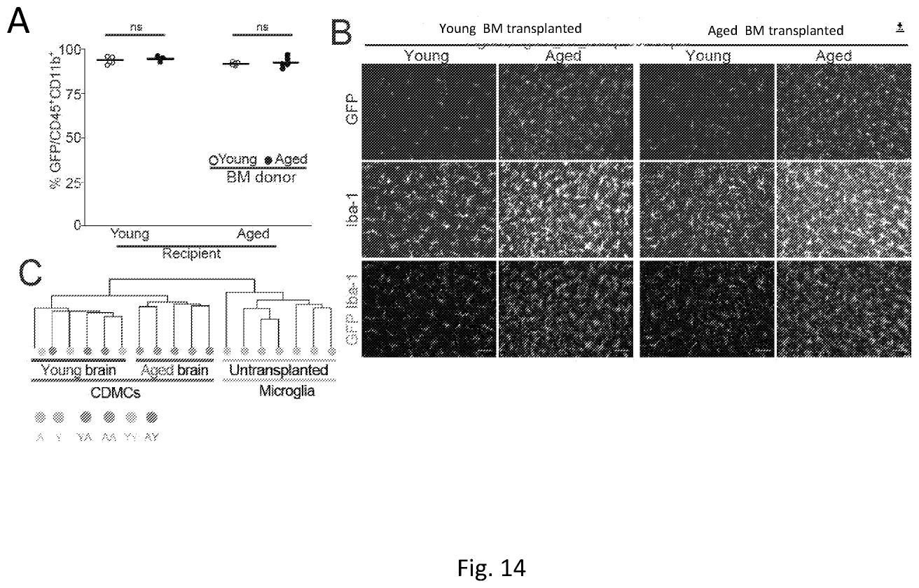

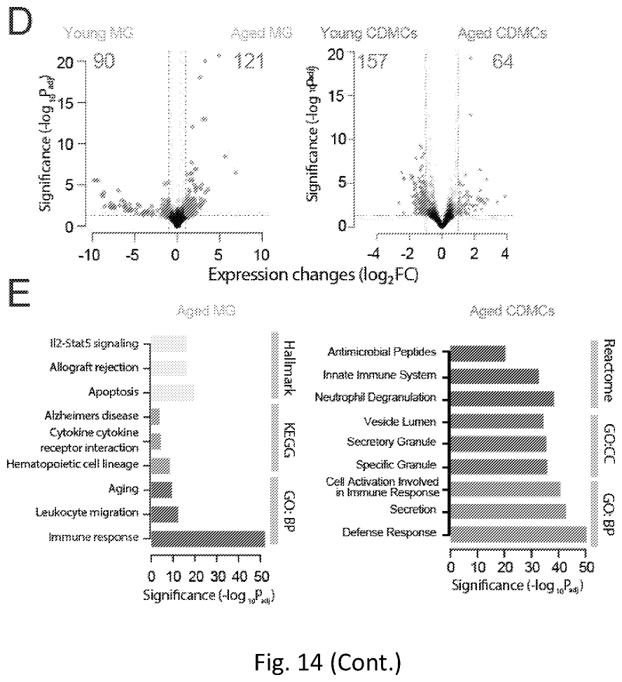

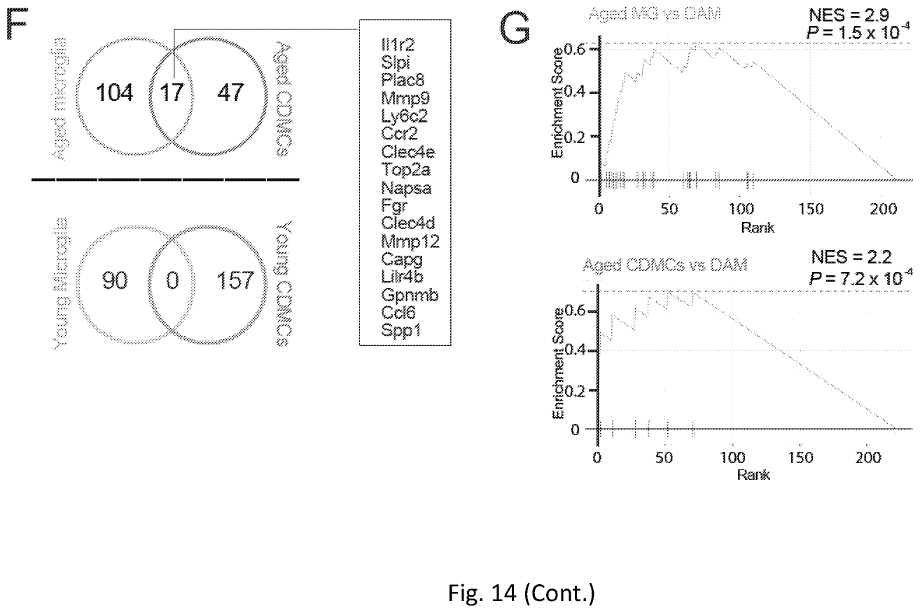

To determine the effect of the brain environment on myeloid cells, we aimed to characterize CDMCs engrafted into the brains of young (2-3 mo) and aged (18-20 mo) recipient mice. Bone marrow was isolated from young (2-3 mo) and aged (18-20 mo) Ubc::EGFP mice and then transplanted each into young and aged WT mice using our HSCT+P protocol (FIG. 7A). The CDMC replacement of all age groups was consistently high and as efficient as with young donor and recipient mice that we had used thus far (FIG. 14A, B). We also obtained untransplanted young (Y) and aged (A) mice as control. We next FACS-isolated CDMCs from the transplanted groups (YY, YA, AY, and AA) 12 weeks after transplantation and microglia from the untransplanted groups (Y and A) at the same time point and performed RNA-seq. As expected, microglia from the untransplanted groups and CDMCs from the transplanted groups clustered separately in the PCA plot and unsupervised hierarchical clustering (FIG. 7B, 14C). Strikingly, among the CDMC samples, the CDMCs in the young brain (YY and AY) and the CDMCs in the aged brain (YA and AA) clustered together regardless of donor age. This finding clearly shows that the host brain environment rather than donor cell age determines the expression profile of engrafted brain myeloid cells.

Given that principal component 2 explained much of the aging-related variation in both microglia and engrafted CDMCs (FIG. 7B), we hypothesized that aged brain environment had similar effects on brain myeloid cells. To test this possibility, we re-grouped the CDMC samples in the young brain (YY and AY) as young CDMCs and in the aged brain (YA and AA) as aged CDMCs and then sought to further characterize CDMCs and microglia in the young and aged brains. GO term analysis of DEGs showed that terms associated with immune response were enriched in both aged microglia and aged CDMCs (FIG. 14D, E). We also found that differentially expressed genes between young and aged CDMCs (FIG. 14D) and expression of these genes in young and aged microglia were positively co-related (FIG. 7C). Next we sought to determine “myeloid aging signature genes” by comparing enriched genes in the aged brain groups.

We identified 17 genes overlapping between aged microglia and CDMCs, whereas no gene overlapped between young microglia and CDMCs (FIG. 14F). We confirmed that the identified 17 genes were highly expressed in the myeloid cells in the aged brain (FIG. 7D). Of note, 3 of the identified 17 genes (Gpnmb, Ccl6, and Spp1) were prominent representatives of the degeneration-associated microglia (DAM) signature genes. This result prompted us to further explore expression of DAM signature genes in our datasets. First, we determined which DAM genes were enriched in our aged groups. We found 26 DAM genes in the total of 208 genes enriched in aged groups (FIG. 7E). Most of these 26 DAM genes were highly expressed in the aged groups with the exception of 6 genes (Apoe, Lyz2, Clec7a, II1b, Cd63 and Cybb) that were only mildly induced in aged microglia and high in all CDMC samples (FIG. 14F). We also performed gene set enrichment analysis (GSEA) and found a correlation between the aged microglia enriched genes and DAM genes (NES=2.9,FDR<0.001) as well as the aged CDMC enriched genes and DAM genes (NES=2.2, FDR<0.001) (FIG. 14G). Morphological analysis of transplanted CDMCs from the 6 different groups recapitulated published findings that aged microglia had less branched and shorter processes and increased cell body to cell area ratio than young microglia (Mosher and Wyss-Coray, 2014) (FIG. 7G, H). In perfect congruence with our RNA-seq results, we further found that both young and aged CDMCs engrafted into the aged brain (YA or AA) had much enhanced morphological aging features than CDMCs engrafted in the young brain (YY and YA). These data demonstrate that, irrespective of donor age, CDMCs rapidly acquire an aged microglia-like morphology and gene expression profile upon the engraftment in the aged brain which resembles a DAM-like expression profile.

In this study, we have developed a highly efficient (>90%) microglia replacement method in mice that does not require any genetic modification of donor or recipient animals. The protocol is robust as it works in all mice tested from various age groups up to 18 months. Microglia are replaced by myeloid cells from the circulation which are stably integrated (up to at least 6 months). We believe this method provides a valuable resource to study engrafted CDMCs and microglia in various contexts and a development platform for cell transplantation-based therapies for the CNS.

Microglia replacement strategies are particularly useful in combination with genetic engineering of donor cells. In mouse models, CDMCs are recruited to the CNS under pathological conditions such as brain or spinal cord injury, stroke, and certain neurodegenerative diseases. CDMCs can be induced in transgenic mouse models of microglia ablation or HSCT mouse models. Recent studies in these models have revealed that CDMCs and microglia have distinct transcriptional signatures in the healthy and young brain. However, it is challenging to study CDMCs in a disease-associated context because 1) genetic models require time- and labor-consuming mouse breeding, and 2) CDMC engraftment after conventional HSCT is a slow and variable process. Given the robust, quick and consistent engraftment of CDMCs in non-transgenic mice, our optimized method can overcome these problems and become a novel platform to study CDMCs in various conditions.

Our method for microglia replacement by HSCT+P has relevant translational implications. It does not require genetic engineering of the recipient and is therefore is applicable to human. The blood brain barrier (BBB) poses a major barrier to treating brain diseases for small molecules, biologics, and cell-based therapies. Since CDMCs have the ability to cross the BBB and engraft throughout the brain, the optimized HSCT method described provides an efficient way to deliver therapeutic agents to the entire brain. Moreover, newly populated CDMCs are long-lived, which enables a sustained therapeutic effect following a one-time treatment in contrast to repetitive drug administrations. Therefore, microglia replacement therapy provides a powerful approach for treating neurological disorders that have global CNS pathology.

In fact, HSCT has been performed for long time to treat lysosomal storage diseases (LSDs) with CNS pathology, aiming to repopulate the recipient myeloid compartment, including microglia, with cells expressing the hydrolase that is defective in the recipient. However, efficacy of HSCT in LSDs depends on the delay between the onset of the disease and the transplantation procedure; HSCT is not effective in a later stage of LSDs with severe CNS pathology. This is likely due to irreversible damage at progressed disease stages but also because microglia replacement after conventional HSCT is inefficient and slow. We showed that >90% brain chimerism could be achieved as early as 34 days after transplantation. Thus, our HSCT+P method can elicit beneficial effects in a mouse model of LSD with severe CNS phenotype.

We also showed that HSCs had the highest capacity to incorporate as CDMCs upon transplantation. This has important practical implications because HSCs are well defined, can be efficiently genetically modified and it was recently found that they can be readily expanded in vitro. This enables the introduction of genetically modified CDMCs into the brain, e.g. to accomplish expression of therapeutic genes. HSCT can have severe side effects including immune depression and chemotherapy-associated toxicity. Development of a safer microglia replacement strategy avoids toxic pre-conditioning, e.g. using recently developed antibody-based preconditioning for HSCT.

We showed that CDMCs and microglia had distinct identities, which is consistent with previous studies. However, we found that CDMCs following HSCT+P were more slightly more similar to microglia than following conventional HSCT based on genome-wide expression and protein levels of some select markers. A similar result was observed in microglia-like cells derived from various hematopoietic cells when these cells were directly injected to the microglia-deficient brain of Csf1r−/− mice. Thus, circulating cells are able to acquire a more microglia-like identity upon exposure to microglia-depleted brain environment achieved by CSF1R inhibition either pharmacologically or genetically. However, opening up microglia niches in the brain does not appear to be sufficient for robust microglia replacement with CDMCs. For instance, following acute microglia depletion using CSF1R inhibitors, the brain is quickly repopulated by new microglia derived from the brain-resident cells but not from circulating cells.

Our data also showed that HSCT pre-conditioning with PLX5622, even in combination with busulfan pre-treatment, did not result in robust engraftment of CDMCs. On the contrary, several transgenic mouse models for microglia ablation allow robust CDMC engraftment in the brain. These results suggest that there appears to be additional mechanisms that recruit CDMCs to the microglia-depleted brain. To overcome the BBB may be one critical component as BBB breakdown allows circulating cells to infiltrate the brain. On the other hand, there is evidence that BBB disruption may not be required for CDMC engraftment into the brain.

During acute or chronic inflammatory reactions, circulating monocytes can infiltrate the brain and differentiate into macrophages. These cells are likely different than CDMCs incorporated after HSCT. We found that HSCs rather than circulating monocytes became incorporated CDMCs most efficiently. This observation is compatible with a previous study that showed that an HSC-enriched but not HSC-depleted cell population migrates to the brain for engraftment shortly after systemic injection and HSCs can give rise to microglia-like cells after direct brain injection. We therefore speculate that HSCs or immediate derivatives are the progenitors of brain CDMCs that enter the brain and then extensively proliferate to replenish the vacant microglia niches. In line with this notion is a recent report that showed clonal expansion of repopulating CDMCs.

We made the remarkable discovery that CDMCs engrafted in the aged brain adopted aged microglia-like features including the upregulation of several DAM signature genes. This result clearly demonstrates that the aged brain environment can induce an aging-like phenotype in myeloid cells. Thus, the aging-related, DAM-like program is likely a reaction to the aged or diseased brain. This notion is supporting the recently proposed model that the DAM phenotype may be a protective immune response against CNS pathology. Our microglia replacement protocol combined with genetic engineering provides an opportunity to enhance such beneficial reactions and combat neurodegenerative disorders.

### Example 2

### Therapeutic Activity of CDMCs in a Mouse Model of Gaucher Disease (GD)

To demonstrate that microglia replacement has therapeutic activity in the brain we chose a second-generation mouse model of Gaucher disease (GD) that shows prominent CNS involvement and better imitates the human disease course than earlier models. The model contains three different kinds of genetic elements: (i) a homozygous Gba1^{D409H} mutation found in patients, a homozygous knock-out of Prosaposin (Psap), a lysosomal co-factor of Gba1-encoded galacto-cerebrosidase, and a Psap transgene, which elevates overall Psap levels to below 50%. Both GBA1 and PSAP mutations cause GD in humans.

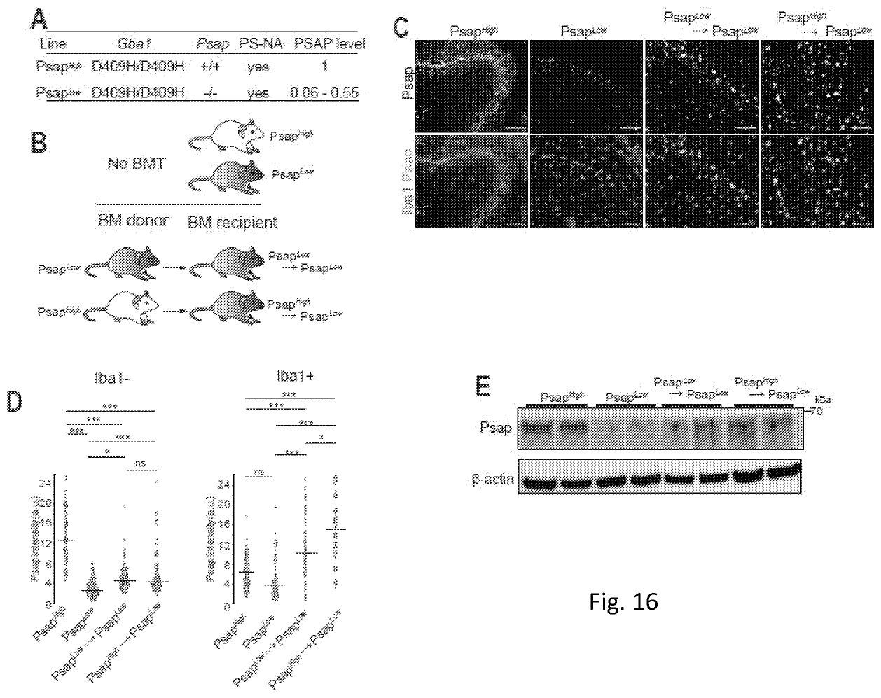

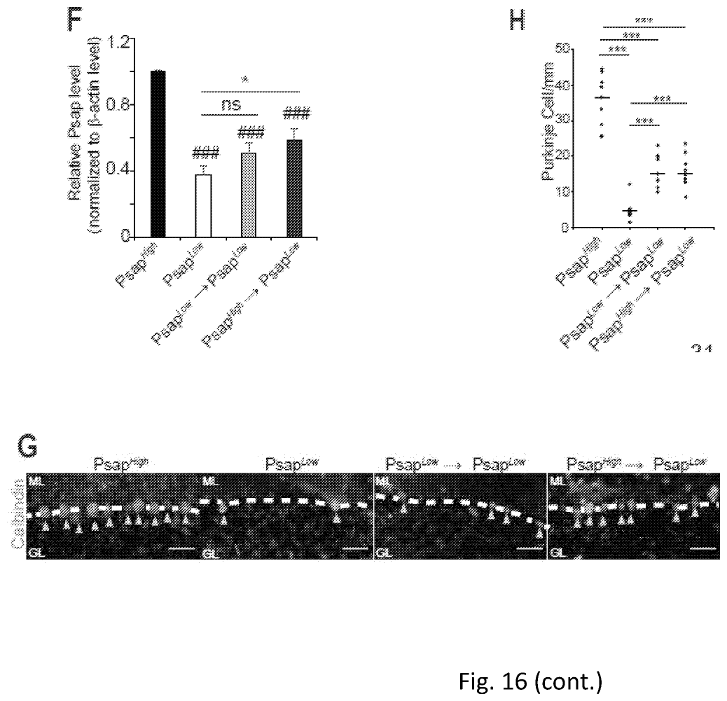

To allow syngeneic transplantation we used littermates as donor mice. Littermate donors available were Gba1^{D409/D409H}; Psap-transgene; Psap^{-/-} (Psap^{Low}) or Gba1^{D409/D409H}; Psap-transgene; Psap^{+/+} (Psap^{High}) (FIG. 16A). Transplanted animals were compared to unmanipulated Psap^{Low} and Psap^{High} mice (FIG. 16B). We did not include a conventional BMT as control because brain chimerism is highly variable in this procedure and therefore results would be inconclusive. To test disease prevention we evaluated microglia replacement at the young adolescent stage of ˜8-9 weeks, at the time of disease symptom initiation. After confirming successful peripheral blood and brain chimerism in this mouse strain using a Y-chromosome-specific droplet digital PCR, we first analyzed levels of Psap in the 4 different conditions at age 15 weeks, i.e. 6 weeks after microglia replacement. Psap immunofluorescence analysis showed that Psap protein expression was greatly reduced in GD mice and increased in both transplant groups (FIG. 16C). However, we also observed that most of the Psap signal in Psap^{Low} and Psap^{High} mice was located in Iba-1 negative compartments whereas in the transplant groups Psap was mostly overlapping with Iba1. Assuming that primarily the microglia-secreted Psap can be therapeutic, we quantified Psap expression inside (Iba-1+) and outside (Iba-1−) microglia separately. These results showed that secreted Psap was indeed elevated in both treatment groups (FIG. 16D, left). Psap staining within Iba-1⁺ cells was elevated in both transplant groups, demonstrating that CDMCs express higher Psap levels from the Psap transgene than microglia (FIG. 16C, right). As expected, Psap^{High} CDMCs expressed higher Psap levels than Psap^{Low} CDMCs. Western blotting and quantification confirmed overall increase of Psap protein levels in the two transplant groups (FIGS. 16E and F). Thus, although CDMCs express higher levels of the Psap transgene than microglia in the GD model, they secrete a smaller fraction of Psap than microglia.

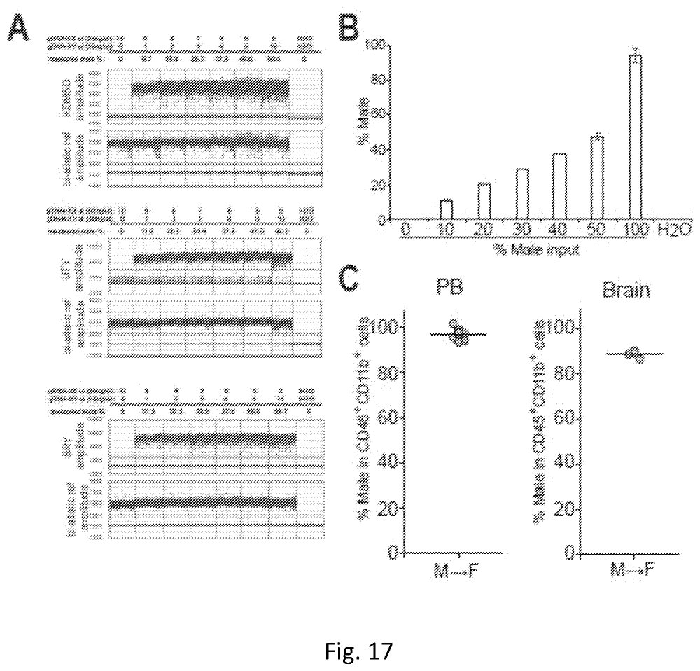

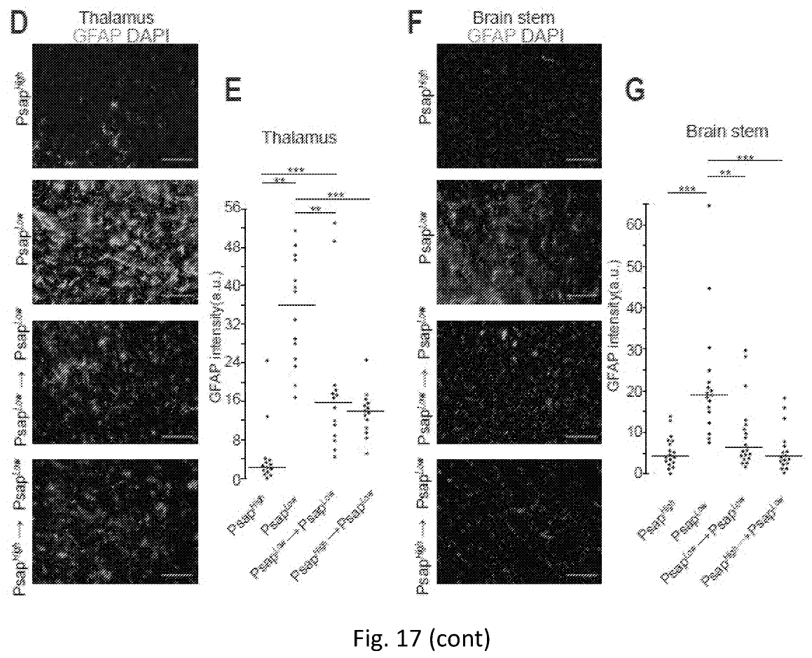

We then evaluated whether the elevated Psap levels observed in the brain has therapeutic effects. One prominent feature of this model is progressive Purkinje cell degeneration and astrogliosis in thalamus and brain stem. Immunofluorescence microscopy and quantification at week 15 showed that more Calbindin-positive Purkinje cells survived in the transplanted animals (FIGS. 16G and H) and astrogliosis was markedly reduced (FIG. 17D-G).

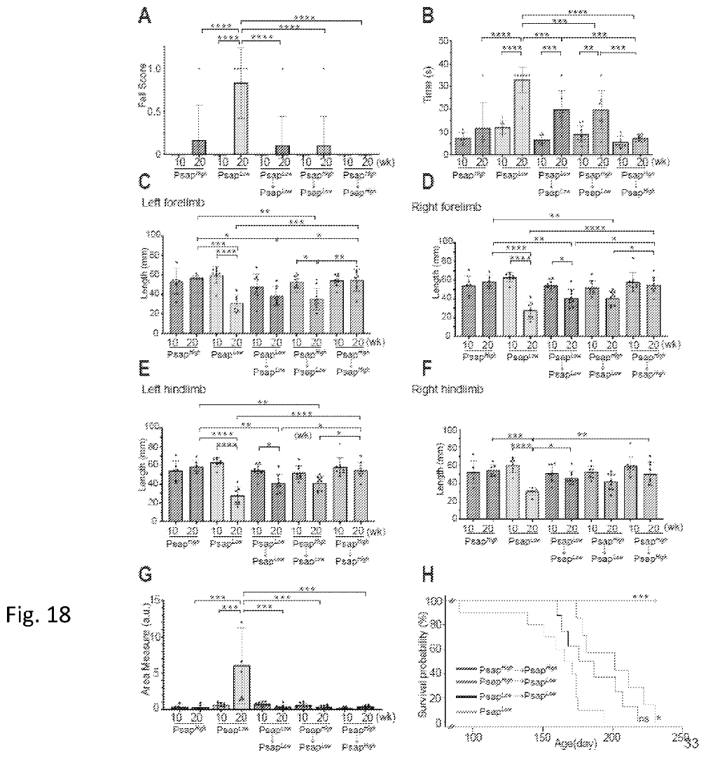

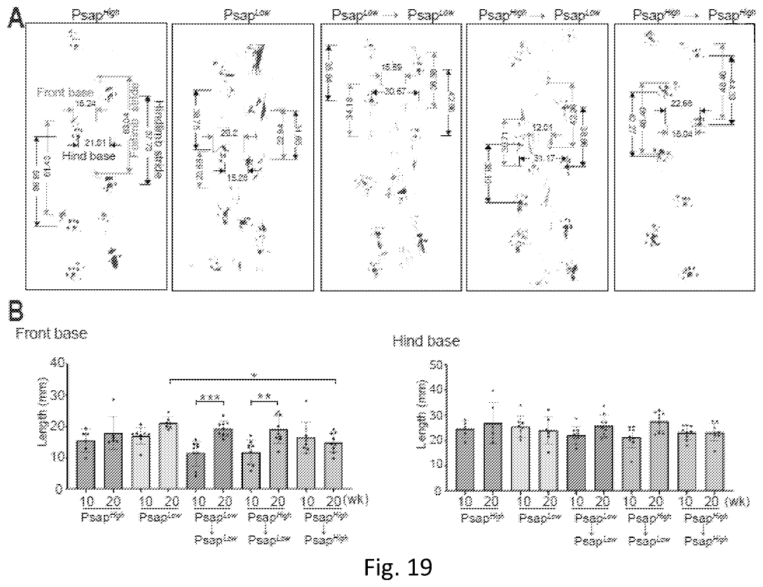

Next, we performed behavioral assessments to assess cerebellar function, i.e. balance, gait, and motor coordination. Reassuringly, both transplant groups showed improved performance in the beam walk test compared to GD mice (FIGS. 18A and B). The fall score at 20 weeks was comparable between treated GD mice and untreated Psap^{High} mice; the time spent on the beam was significantly reduced in the treated mice but did not reach control levels. Gait coordination was assessed by footprint analysis (FIG. 18A). Again, both treatment groups showed a significant improvement of the ataxic gait that progresses in GD mice (FIG. 18B, FIG. 16C-F). Finally, we examined general exploratory and locomotor activity in these mice by the open field test. GD mice showed a significant increase in Area Measure (a metric of the number of sharp turns instead of straight-line runs) over time whereas no such increase was observed in treated mice (FIG. 18G). In line with the behavioral improvements, the overall survival rate was improved in both treatment groups; GD mice transplanted with Psap^{High} cells survived significantly longer than untransplanted GD mice by 19.3%, GD mice transplanted with Psap^{Low} donor cells trended to survive longer by 7.4% (FIG. 16H). Importantly, the BMT+P procedure itself did not affect any of these behavioral parameters as Psap^{High}→Psap^{High} transplanted animals did not show any abnormalities (FIG. 16). In summary, these results demonstrate that our high-efficiency microglia replacement BMT+P protocol increases Psap levels in the brain which leads to a therapeutic benefit in GD mice including a significant increase of survival.

Gaucher disease is the most common lysosome storage disease with an incidence of 1/40,000 live births, caused by decreased function of the lysosomal enzyme galacto-cerebrosidase (GCase), encoded by the GBA1 gene. Prosaposin is a microglial secreted and endo-lysosomal precursor protein that is cleaved into saposin C which functions as a critical co-factor for GCase activity. Hence, mutations in PSAP also cause GD. ERT is highly effective for type 1 disease affecting primarily non-CNS organs. Importantly, however, ERT is ineffective for type 2 and 3 disease, which are characterized by dominant CNS involvement. Moreover, as ERT increases life expectancy, CNS involvement in type 1 patients becomes more prominent. Thus, there is an urgent clinical need to treat CNS pathology in GD.

A novel and highly efficient microglia cell replacement that provides therapeutic benefit for a pre-clinical model of GD is provided herein. We utilized a new generation mouse model of neuronopathic GD that exhibits pronounced CNS pathology including Purkinje cell degeneration, astrogliosis, progressive cerebellar dysfunction, and premature death. Just like in human disease, peripheral enzyme replacement therapy has no therapeutic effects and does not improve survival in a related Psap/Gba1 model. Thus, together with challenging efforts to generate brain-permeable enzymes the cell therapy disclosed herein is the only current approach to affect CNS pathology. Of note, we demonstrate that microglia replacement was able to reduce CNS pathology even when performed at around the time of disease onset, a timing more clinically realistic than prospective treatment. However, as newborn screening programs for lysosomal storage diseases become more prevalent, earlier treatment may become feasible with likely larger therapeutic effects. The finding that microglia replacement can rectify CNS pathology is highly relevant for neural regeneration and provides a blueprint for cell-based brain therapies that could be applied to a large variety of neurological diseases. Microglia are at the epicenter of regulating the pathogenesis of many diseases including neurodegeneration, traumatic injury, auto-immune disease, vascular brain insults, inflammatory disease, and brain tumors.

Another insight from this study is that primary HSCs can be used as donor cells for high efficiency microglia replacement. Using HSCs as donor cells has some practical advantages as they are well defined, can be expanded in culture several hundred-fold and genetically manipulated by efficient lentiviral gene transfer allowing the ex vivo genetic engineering of donor cells to bestow transplanted cells with additional therapeutic properties. HSC-like cells or other hematopoietic cells with equivalent microglia repopulating properties are generatable from induced pluripotent stem (iPS) cells which can be derived from patients in an autologous fashion and are amenable to an even wider spectrum of genetic engineering including therapeutic gene targeting by homologous recombination. The combination of microglia replacement with genetic donor cell engineering represents a versatile approach to interfere with brain pathology.

One experimental arm, the transplantation of Psap^{Low} donor cells to Psap^{Low} mice was therapeutic because the transplanted CDMCs expressed higher levels of the Psap transgene than endogenous microglia. These results demonstrate the intriguing possibility that under certain conditions even transplantation with unaltered mutant cells can increase therapeutic gene expression in case the gene of interest happens to be expressed at higher levels in CDMCs than microglia. This scenario is applicable e.g. for known hypomorphic mutations whose increased expression is expected to be therapeutic. For this purpose it is can be beneficial that CDMCs are transcriptionally distinct from microglia, independent of their method of integration.

In summary, this paper demonstrates microglial cells as effective cellular target for cell-based therapies for regenerative efforts to restore brain function. In combination with genetic engineering and the ability to transdifferentiate microglia into neural cells microglia replacement allows the efficient delivery of therapeutic agents into the brain and may even regenerate lost brain cells.

### Example 3

### Genetic Manipulation of Cultured and Expanded HSCs and Sustained Transgene Expression in HSC-Derived CDMCs

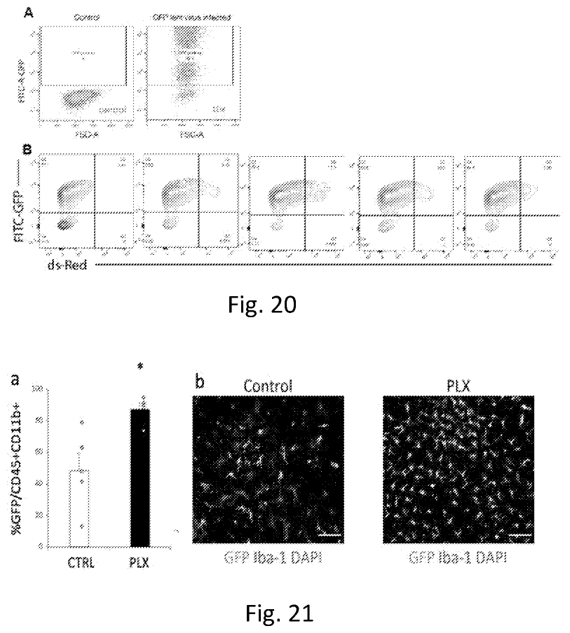

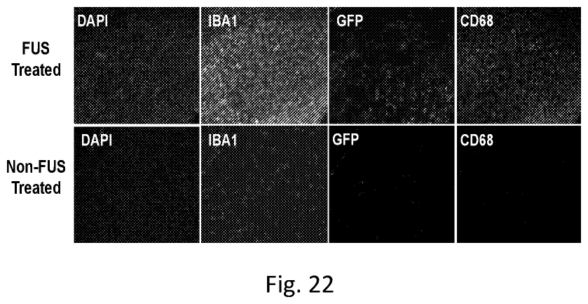

Given that HSCs are the most potent hematopoietic cell population that gives rise to CDMCs equivalent to WBM cells, we tested whether HSCs could be genetically manipulated to eventually express therapeutic genes. To that end, we cultured and expanded HSCs. We then infected the cells in vitro with a lentivirus encoding the enhanced green fluorescent protein (EGFP) and found stable and high expression levels in vitro (FIG. 20A). To assess whether additional transgenes can be delivered to HSCs and whether such exogenously expressed transgenes would be stably expressed even after transplantation and incorporation into the CNS as CDMCs, we transplanted expanded HSCs infected with lentiviruses encoding a gene for the red fluorescent protein dsRed and performed the BMT/PLX protocol as outlined above. Indeed, robust dsRed fluorescence could be detected 7 weeks after transplantation (FIG. 20B)

### Example 4

### Efficient Microglia Replacement in the Spinal Cord

Many CNS diseases affect primarily the spinal cord. To evaluate whether our microglia replacement protocol is truly CNS-wide we characterized the spinal cords following a replacement as described above. Indeed, when using GFP-expressing donor cells we were able to identify a high and robust chimerism of GFP-positive, transplanted cell among the myeloid cells of the spinal cord (FIG. 21),

Microglia Replacement without Toxic Preconditioning.

Busulfan or irradiation are mostly used in the clinic to condition the bone marrow, in order to allow hematopoietic stem cell engraftment. Both regimens also pre-condition the brain, as they allow low level and variable chimerism to establish within the myeloid compartment of the brain. We established that such brain conditioning is essential for microglia replacement since hematopoietic engraftment mediated by Anti-c-kit antibodies conjugated to Saposin leads to exchange of the peripheral blood but not in the CNS. We therefore determined whether it is sufficient to transiently disrupt the blood brain barrier (BBB) to allow cell entry and replacement of endogenous microglia with CDMCs.

MRI Guided Focused Ultrasound (MRgFUS) safely and non-invasively opens the BBB in a transient manner, see for example Chopra et al. (2010) ACS Chem Neurosci. 1(5):391-8. The technique utilizes a microbubble suspension prior to FUS exposure. The varying pressure field of the FUS generates controlled oscillation of the bubbles which interact with endothelial cells, compromising the integrity of the tight junctions. This disruption enhances delivery of chemotherapeutic agents, monoclonal antibodies, neurotrophic factors, and stem cells. It has been used in many animal models, such as brain tumors and Alzheimer's disease, to deliver various substrates including antibodies and stem cells. We have performed experiments which show that circulating cells can migrate into the brain following MR-g FUS. We first replaced the hematopoietic system using non-toxic antibody preconditioning with GFP+ cells. Once confirmed that GFP chimerism was over 90% in the peripheral blood, we applied MRgFUS in those mice. Indeed, the treated hemisphere showed substantial ingression of GFP+ cells into the brain parenchyma whereas in the control, untreated hemisphere of the same animal literally no GFP+cells were detectable. Many of the invading GFP+ cells were immune reactive with the microglial markers Iba1 and CD68.

### Experimental Model and Subject Details

Mouse. All animal procedures were approved by the administrative panel on laboratory animal care at Stanford University. All mice were age and sex matched (except for breeding pairs) and group-housed (up to 5 mice per cage) on a 12 h/12 h light/dark cycle with water and standard chow ad libitum. For PLX5622 treatment, mice were provided PLX5622-incorporated diet for the indicated time period in the text. All mice used in this study appeared healthy. Unless otherwise noted, we used female mice at age of 8-12 weeks. Number of animals used in each experiment are described in the figures or figure legends. All mice were immunocompetent and on a C57BL/6 genetic background.

C57BL/6 (WT, 2-4 months old), Gfp-C57BL/6-Tg(UBC-GFP)30Scha/J (Ubc::EGFP, 2-4 months old), B6.129P2(Cg)-Cx3cr1^{tm2.1(cre/ERT2)Litt}/WganJ (Cx3cr1::CreER), and B6.Cg-Gt(ROSA)26Sor^{tm14(CAG-tdTomato)Hze}/J mice (Rosa26::lox::Stop::lox::tdTomato) were obtained from the Jackson Laboratory. Aged WT mice (18-20 months old) were obtained from the National Institute on Aging rodent colony. Aged Ubc::EGFP mice (18-20 months old) were prepared in house. Cx3cr1::CreER; Rosa26::lox::Stop::lox::tdTomato; Ubc::EGFP mice (all transgenes were hemizygous) were obtained by crossing the Cx3cr1::CreER, Rosa26::lox::Stop::lox::tdTomato, and Ubc::EGFP mouse lines in house as follows. First, we crossed the Cx3cr1::CreER line to Rosa26::lox::Stop::lox::tdTomato line to obtain Cx3cr1::CreER (+/−); Rosa26::lox::Stop::lox::tdTomato (+/−) mice. We next crossed these F1 generation mice to Ubc::EGFP (+/+) to obtain Cx3cr1::CreER (+/−); Rosa26::lox::Stop::lox::tdTomato (+/−); Ubc::EGFP (+/−) mice. KuO mice were on a C56BL/6 background and created as described previously (Hamanaka et al., 2013). Genotyping was performed by standard PCR using tail tip samples.

We utilized the previously established PS-NA/Gba1 mouse model of Gaucher's (Sun et al., 2005). This line contains a D409H/D409H point mutation in Gba1 that leads to <5% normal glucocerebrosidase activity. In addition, they contain the prosaposin (PS) knockout (PS−/−) mice, termed GD, containing the modified prosaposin transgene and named PS-NA (Y et al., 2008). PS-NA transgene has 4-45% of saposin protein expression in brain. The healthy littermates have genotype D409H/D409H; PS+/+ NA, termed CTRL. The mice have a mixed genetic background of approximately 50% FVB, 25% C57BL/6, and 25% 129SvEvBrd. Mutant mice develop ataxia and waddle gait. To obtain GD and CTRL mice, we set up D409H/D409H; PS+/−NA breeding pairs and genotyped for the PS gene.

PCR Primers Used were:

Cx3cr1::CreER (Common F 5′-AAG ACT CAC GTG GAC CTG CT-3′, Mutant R1 5′-CGG TTA TTC AAC TTG CAC CA-3′, WT R2 5′-AGG ATG TTG ACT TCC GAG TTG-3′) Rosa26::lox::Stop::lox::tdTomato (WT F 5′-AAG GGA GCT GCA GTG GAG TA-3′, WT R 5′-CCG AAA ATC TGT GGG AAG TC-3′, Mutant F 5′-CTG TTC CTG TAC GGC ATG G-3′ Mutant R 5′-GGC ATT AAA GCA GCG TAT CC-3′). PS-NA/Gba1 (PS F 5′-TTC AGC AAG TTC CCA GCT TCG G-3′, PS R 5′ GAG CCC AAT TTT AGC AAG AGA 3′).

PCR Program

1. 94.0° C. (2 m), 2. 94° C. (20s), 3. 65° C. (−0.5° C. per cycle, 15s), 4. 68° C. (10s), 5. Repeat steps 2-4 for 10 cycles (Touchdown), 6. 94° C. (15s), 7. 60° C. (15s), 8. 72° C. (10s), 9. Repeat steps 6-8 for 28 cycles, 10. 72° C. (2 m). For the Cx3cr1::CreER transgene, expected results are Mutant(+/+)=−300 bp, Heterozygote (+/−)=−300 bp and 695 bp, WT(−/−)=695 bp For the Rosa26::lox::Stop::lox::tdTomato transgene, expected results are Mutant (+)=−200 bp WT (−)=297 bp. PS-NA/Gba1. 1. 94.0° C. (3 m), 2. 94° C. (1 m), 3. 60° C. (2 m), 4. 72° C. (2 m), 5. Repeat steps 2-4 for 35 cycles. For the PS gene, expected results are WT(+/+)=−350 bp, Heterozygote (+/−)=−350 bp and 1.5K bp, knockout (−/−)=1.5K bp

### Method Details

PLX5622 treatment.

PLX5622 was provided by Plexxikon Inc. and incorporated to AIN-76A standard chow by Research Diets Inc. at 1200 mg/kg. Mice were treated with PLX5622 or control diet ad libitum for the duration indicated in the figure legends.

Bone Marrow Transplantation.

Unless otherwise noted, we used female mice at age of 8-12 weeks for both recipients and donors. WT mice were pre-treated with PLX5622 diet between day-21 and day −1 (21 days) and/or busulfan between day-6 and −2 day (5 days). For busulfan pre-treatment, mice received five intraperitoneal (IP) injections of busulfan (sigma, B1 170000) at 25 mg/kg body weight per day. On day 0, bone marrow cells (BMCs) were isolated from tibias and femurs of UBC-GFP mice. Isolated BMCs were resuspended in 1×red blood cell lysis buffer (e-bioscience, 00-4300-54) and incubated for 20 min at room temperature. BMCs were passed through 70 μm cell strainer, washed by applying 10 ml of ice cold PBS and then resuspended in fresh PBS. BMCs (5×10\hat{ }6) in 200 ul PBS were injected into the retro-orbital sinus of the pre-conditioned recipients. On day 28, the recipient mice were treated with PLX5622 or control diet for 10 days. For tamoxifen injection, tamoxifen (sigma T5648) in corn oil (sigma, C8267) was provided by IP injection to the recipient mice at 200 mg/kg at the indicated time points after bone marrow transplantation.

Microglia Isolation for Flow Cytometry.

Microglia cells were isolated from adult mouse brains as described (Shibuya et al., 2014). Anesthetized mice (100 mg/kg of ketamin and 10 mg/kg of xylazine, i.p.) were perfused through the left ventricle with ice-cold PBS without Ca²⁺ and Mg²⁺. Brains were removed and cut into two pieces along the midline. One hemisphere was immediately transferred to 4% paraformaldehyde for fixation. The other hemi-sphere was minced with a surgical knife, and then transferred to ice-cold HBSS without Ca²⁺ and Mg²⁺. Minced hemi-brains were centrifuged at 300×g for 2 min at room temperature and then enzymatically digested at 37° C. for 35 min by using Neural Tissue Dissociation Kit (Miltenyi Biotec, 130-092-628) according to the manufacturer's protocol. The cell suspension was passed through a 70 m cell strainer and washed by applying 10 ml of HBSS with Ca²⁺ and Mg²⁺ to the strainer. The cell suspension was centrifuged at 300×g for 10 min at room temperature, re-suspended in 30% Percoll (GE Healthcare Lifesciences, 17089102), and then centrifuged at 700×g for 10 min at 4° C. The supernatant containing the myelin was removed and the pellet was washed with 10 ml of HBSS with Ca²⁺ and Mg²⁺. The resulting pellet was resuspended in FACS buffer and subjected to antibody staining for flow cytometry. Microglia/CDMCs were identified using anti-CD45 and anti-CD11b antibodies.

Flow Cytometry.

The following FACS antibodies were used in this study. CD11b (clone M1/70, BioLegend), CD45 (Clone 30-F11, BD biosciences), F4/80 (clone BM8, biolegend), CCR2 (clone SA203G11, biolegend), gp49A/B (clone H1.1), CD115 (CSF1R) (clone AFS98, biolegend), CD117 (c-kit) (clone 2B8, BD biosciences), sca-1 (clone D7, biolegend), cd127 (IL7Ra)(clone SB/199, biolegend), CD16/CD32 (FcR Ill/II) (clone 93, biolegend), CX3CR1 (clone K0124E1, biolegend), cd135 (Flk-2,)(clone A2F10) CD34 (clone561, biolegend), and Ly6C (clone RB6-8C5). Cells were suspended in FACS buffer (5% FBS in PBS) and stained with FACS antibodies for 15-90 min at 4° C. Cells were then washed with FACS buffer and sorted using a FACS AriaII. DAPI was used as a dead stain. Post-acquisition analysis was performed using FlowJo software.

Immunofluorescence microscopy. The following primary antibodies were used in this study. Iba-1 (FUJIFILM Wako Pure Chemical, 019-19741), GFP (abcam, ab13970), CD68 (biolegend, 137001), TMEM119 (abcam, ab209064) and Ki-67 (thermo fisher, 14-5698-82). Anesthetized mice (100 mg/kg of ketamin and 10 mg/kg of xylazine, IP) were perfused through the left ventricle with ice-cold PBS without Ca²⁺ and Mg²⁺. The brain was isolated, transferred to 4% paraformaldehyde immediately and then fixed overnight at 4° C. followed by cryo-protection with 30% sucrose (in PBS) solution. Brains were then cryo-embedded with Tissue-Tek OCT compound (Sakura Finetek, 4583), and sagittal sections (40 μm) were obtained using a cryostat (CM 3050S, Leica). The sections were washed 4 times with PBS and processed for antibody staining. Brain sections were permeabilized and blocked with blocking buffer (0.3% Triton X-100 (Sigma, X-100) and 5% cosmic calf serum in PBS) for 1 h. Sections were incubated with primary antibodies in blocking buffer either for 1 h at room temperature or overnight at 4° C. Brain sections were washed 3 times with PBS and then incubated with AlexaFluor dye-conjugated secondary antibodies in blocking buffer for 1 h at room temperature. Sections were washed 3 times with PBS and then incubated with DAPI in PBS for 2 min at room temperature. Sections were washed once with PBS and mounted on glass slides with a drop of ProLong Gold antifade reagent (Thermo Fisher, P36930). Images were obtained using a Zeiss LSM710 confocal microscope or Zeiss AxioImager motorized widefield fluorescence microscope.

Image analysis. All image analyses were performed using ImageJ (NIH, USA) software. For morphological analyses, Iba1+ cells and GFP+ cells in the indicated brain regions were analyzed. The number of Iba1+ cells, and number of branch points were manually counted using ImageJ. Cells with more than two processes were considered “ramified” in the analysis. The length of processes in Iba-1+ cells was analyzed semi-automatically using NeuronJ plugin. The ratio of cell body to total cell area was analyzed using FracLac plugin as described. Briefly, individual Iba-1+ cells were made binary following removal of digital noise. Binary images were converted to outlines using ImageJ and then processed by FracLac plugin. The resulting convex hull images were used to calculate the ratio of cell body to total cell area.

Stereotaxic Injection of Bone Marrow Cells.

WT mice were pre-treated with or without PLX5622 diet for 20 days. On day 0, donor bone marrow cells were freshly isolated from Ubc::GFP mice as described above and then stereotaxically injected to the brain of pre-conditioned WT mice. Mice were deeply anesthetized with 100 mg/kg of ketamin and 10 mg/kg of xylazine (IP) and positioned on a stereotactic frame. A stainless steel needle (30 G, Hamilton) was inserted into the hippocampus using the following coordinates from bregma: −2.2 mm anterior, 1.4 mm lateral, and 2.1 mm depth. A total of 100,000 cells/hemibrain in 1 μl PBS was injected at a rate of 0.5 μl/min, and the needle was left in place for an additional 2 min at the end of injection. Ketoprofen (5 mg/kg) was used for postoperative analgesia. Mice were sacrificed for analysis 14 days after injection.

In Vitro Phagocytosis Assay.

Cell suspension was prepared from the mouse brain as described in microglia isolation for flow cytometry. A small portion of the suspension was stained with anti-CD11b antibody for 15 min at 4° C. and subjected to flow cytometry to estimate % phagocytes (CD11b+ cells) in the cell suspension. The suspension was centrifuged at 300×g for 5 min at 4° C., resuspended in ice-cold serum-free DMEM/F-12 (Thermo Fisher, 11320033) at an equal phagocyte concentration and then placed on ice. pHrodo™ Red Zymosan Bioparticles (Thermo Fisher, P35364) were added to the cell suspension at 5 ug/10,000 live phagocytes. The cell-particle mixture was then incubated in a water bath at 37° C. with periodic agitation. At the indicated time point, phagocytosis was halted by transferring the mixture to ice. Anti-CD45 and CD11b antibodies were added to the mixture and incubated on ice for 30 min. The cell-particle mixture was washed at 4° C. with ice-cold FACS buffer and internalized bioparticles in microglia/CDMCs were analyzed by flow cytometry.

RNA Sequencing.

Single cell suspension was prepared from the mouse brain as described above. Approximately 30,000 microglia and CDMCs per hemi-brain were identified by detecting expression of CD45, CD11b, and GFP using a FACS Aria II and then sorted into 700 ul of Trizol LS (thermo fisher, 10296010). Total RNA was extracted and then purified using a Qiagen RNeasy MinElute Cleanup Kit (Qiagen, 74204) according to the manufacturer's protocols. Quality and quantity of RNA was assessed on a Bioanalyzer (Agilent), and samples with RNA integrity number (RIN) >8 were used for subsequent library preparation. The NEBNext Single Cell/Low Input RNA Library Prep Kit for Illumina (New England Biolabs, E6420S) was used to generate cDNA and libraries according to the manufacturer's protocol. The libraries were indexed using the NEBNext Multiplex Oligos for Illumina (New England Biolabs, E6420S) and cleaned up using SPRI select reagent (Beckman Beckman Coulter, B23317). The indexed libraries were pooled and sequenced on the Illumina platform. 150 bp paired-end reads were generated producing a mean of 46.9 million reads per sample. Raw sequencing reads were demultiplexed with bcl2fastq and aligned to mouse transcriptome (mm10; GRCm38.p6) using STAR (version 2.5.2b) with the default parameters. Uniquely mapped reads were 70.25%; reads mapped to multiple loci 2.46%; reads mapped to too many loci 0.43%; reads unmapped (too short) 26.21%; reads unmapped (other) 0.65%. The expression level of Gencode-annotated was calculated using HTSeq-count. Raw counts of each gene were normalized, and genes with low counts were removed using the mean of normalized counts as a filter statistic using the DESeq2 with standard parameters. The Molecular Signatures Database (MSigDB) was used to access enrichment of significantly up- or downregulated genes from RNA-seq analysis in Gene Ontology sets and KEGG pathways. The raw RNA-seq datasets of Bennett et al., 2018 were downloaded from Sequence Read Archive (accession: PRJNA453419), and analyzed using identical analysis pipeline as described above. To remove variability of datasets each generated from different platform, the batch effect correction was performed using limma (version 3.10)

Hematopoietic Cell Isolation and Transplantation.

HSC, CMP, GMP, MEP, and MDP/CDP were isolated as described (Yamamoto et al., 2018) with some modifications. Bone marrow cells were isolated from tibias and femur of UBC-GFP mice at 8-12 weeks of age. After red blood cell lysis with, bone marrow cells were stained for 30 min with APC-c-kit antibody at 4° C., wash once with FACS buffer, and then incubated for 15 min with anti-APC magnetic beads (Miltenyi Biotec, 130-090-855) at 4° C. After a wash step with FACS buffer, c-kit positive cells were enriched using the autoMACS Pro Separator. The c-kit enriched cells were incubated for 30 min with Lineage Cell Detection Cocktail-Biotin (myltenyi Biotech, 130-092-613) at 4° C., wash once with FACS buffer and then stained for 90 min with the indicated FACS antibodies as well as APC/Cy7 Streptavidin (BD biosciences, 554063). After a wash step, the cells were subjected to flow cytometry as described above. Bone marrow monocytes and circulating monocytes were isolated using anti-Ly6C antibody from bone marrow cells and peripheral blood, respectively. For isolation of spleen CD11b+ cells, the spleen was isolated from the same donor mice following bone marrow cell isolation as described above. The spleen was placed on a 70 μm cell strainer put on a 50 ml tube and mashed through the cell strainer using a plunger (BD309604). After rinsing the cell strainer with FACS buffer, cell suspension was centrifuged at 800×g for 5 min at 4° C. Spleen CD11b+ cells were isolated from the cell suspension by FACS.

Pb Analysis.

Approximately 50 ul of peripheral blood per mouse was collected from the facial vein into a 1.5 ml tubes containing 20ul of 0.5M EDTA. Erythrocytes were removed with 1×red blood lysis buffer as described above and cells were washed once with FACS buffer. The resulting cells were analyzed by flow cytometry.

Acute Cortical Window Surgery and Two Photon (2P) Imaging of Cell Dynamics.

2P-imaging experiments were conducted to observe microglia and CDMCs in real time in vivo. Microglia were imaged in adult heterozygous CX3CR1-GFP mice (>8 weeks). CDMCs were visualized in adult WT that underwent busulfan-preconditioned bone marrow transplantation followed by PLX5622 treatment using freshly isolated whole bone marrow from heterozygous CX3CR1-GFP mice. Acute cortical window implantation was performed under ketamine and xylazine anesthesia (100 mg/kg body weight and 10 mg/kg body weight in 0.9% saline via IP injection) and otherwise as described previously (Mohr et al., 2020). The skin atop the skull was retracted, the sutures of the frontal and parietal bones were exposed and sealed with cyanoacrylate glue. A −4.5 mm craniotomy (centered 3.5 mm lateral and 0.5 mm rostral of lambda) was performed over visual cortex, leaving the dura and superficial blood vessels intact. The craniotomy was covered with a 5 mm diameter #1 cover glass fixed in place with cyanoacrylate glue and a custom milled head-bar was attached using dental cement. The head bar was held by a custom milled holder and the window was leveled by reflecting a visible laser spot off the glass surface. In vivo 2P-imaging was performed with a two-photon microscope (Prairie Technologies) and a Nikon 16× water-immersion objective (IR, N.A.=0.8). Fluorescence excitation was achieved through a Ti:sapphire laser (Chameleon, Coherent) tuned to 940 nm, attenuated through a Pockels Cell (ConOptics) to 75 mW post-objective power and emission was filtered with a 580 DCXR dichroic and hq525/70 m-2p bandpass filter. Z-stacks consisting of 26 planes from approximately 150-200 μm below the dura were acquired using identical imaging parameters every 63 sec with 3.0× optical zoom yielding ˜280×280×50 μm image volumes. Individual Z-stacks were sum intensity projected in Z and process length was determined manually for every time point using ImageJ.

Quantitative RT-PCR (qPCR).

A total of 100 ng of total RNA was reverse-transcribed using SuperScript III reverse transcriptase (ThermoFisher, 18080051) to prepare cDNA. qPCR was performed by using SYBR Master Mix (ThermoFisher, 4312704) and a 7900HT fast real-time PCR system (Applied Biosystems). The following cycles were performed: an initial denaturation cycle of 94° C. for 5 min, followed by 40 amplification cycles of 94° C. for 15 s and 60° C. for 1 min). The transcript levels were determined after normalization against Gapdh as described in (Pfaffl et al., 2002). Primers were chosen from PrimerBank.

Droplet Digital PCR.

Genomic DNA (gDNA) was purified from cells using the Quick DNA miniprep kit from Zymo Research (D3025). gDNA was eluted in nuclease-free water (Ambion AM9937). The concentration of gDNA was determined with a Nanodrop Spectrophotometer ND-1000 and adjusted to indicated concentrations with nuclease-free water. gDNA was used as a template for droplet digital (dd) PCR according to manufacturer's instructions for the BioRad QX200 system. Briefly, ddPCR reactions were prepared using 2×ddPCR supermix (BioRad 1863025), bi-allelic reference-HEX primer/probe mix (IDT PrimeTime Std qPCR Assay: primer/probe ratio: 3.6, Primer 1: GGATGGGGAACGCAGCTCTT, Primer 2: AGTGCGGCAGAATACAGCA, probe:/5HEX/TGATGGGTT/ZEN/GTGAAGGCAGCTGCACCT/3IABkFQ/), and one of the following FAM-conjugated primer/probe mixes (all sourced from IDT as PrimeTime Std qPCR Assay; primer/probe ratio: 3.6): KDM5D-FAM (Primer 1: TTCTTCCAATTTCTCTACAGC, Primer 2: CATATACTGTAAAGACTAGGAG, probe: /56-FAM/TGGACACCC/ZEN/CACCACAGCCAA/3IABkFQ/), or UTY-FAM (Primer 1: CTGTTAACATAAATATTGGTCCAG, Primer 2: CACAGAAGTCATTCAGAACAC, probe: /56-FAM/CTTCAGGTA/ZEN/CAACAAACCATTCACAATC/3IABkFQ/), or SRY-FAM (Primer 1: CCTACACAGAGAGAAATACCC, Primer 2: CAACTGCAGGCTGTAAAATGC, probe: /56-FAM/TCAGCCTCA/ZEN/TCGGAGGGCTAAAGTGTC/3IABkFQ/). After generation of droplets in the BioRad QX200 generator, which employed DG8 cartridges (BioRad 1864008), gaskets (BioRad 1863009), and droplet generation oil for probes (BioRad 1863005), ddPCR mixes were loaded into 96 well plates (Fisher Scientific E951020346) and sealed with pierceable foil heat seal (BioRad 1814040). PCR reactions were run in a thermocycler using the following parameters: 1×10 minutes at 95 C, 41×30 seconds at 94 C followed by 1 minute 5 seconds at 58 C (SRY & UTY assays) or 56 C (KDM5D assays), and 1×10 minutes at 98 C. Subsequently, ddPCR reactions were analyzed in a BioRad QX200 droplet reader and data analysis was performed with BioRad's QuantaSoft™ Analysis Pro 1.0 software. The percentage of male chimerism in female background was calculated via the following formula: ((concentration in copies per ul of KDM5D, SRY, or UTY-FAM signal*100)/concentration in copies per ul of bi-allelic reference-HEX signal)*2=% male. More than 10000 droplets were analyzed per sample.

Behavioral Tests.

The genetic background of the mice and transplant treatment were coded and blinded to the experimenters.

Y-maze.

The Y-maze was performed as described in (Faizi et al., 2012). The Y-maze was made of solid white plastic and consisted of two symmetrical arms and one longer arm at 120° angles (longer arm, 20.7 cm length×12.7 cm height×7.62 cm width; equal arms, 15.24 cm length×12.7 cm height×7.62 cm width). At the beginning of trials, mice were placed in the center of the maze and allowed to freely explore the three arms for 5 min. Arm entry was defined as having all four limbs inside an arm. The maze was cleaned with 10% ethanol between animals and before the first animal to eliminate traces of odor. The number of arm entries and the number of triads were recorded to calculate the alternation percentage, which was calculated by dividing the number of triads by the number of possible alternations multiplied by 100.

Open Field Test.

Mouse locomotor behavior was assessed using the force-plate actometer as previously described (Fowler et al., 2001) Mice were individually placed on the 28×28 cm plate surrounded by plastic walls in a well-lit room and allowed to freely explore for 20 min. Spatial statistic, total distance travel, area measure, and time spent in the center of the square were quantified as previously described (Fowler et al., 2001)

Elevated Beam Test.

Mice were assessed on circular beams of decreasing diameter (17 mm and 11 mm). Beams were horizontal and 50 cm above the table. Beams were 100 cm in length with a dark goal box at the end terminus. A bright light illuminates the start terminus. Five days before testing, mice were individually placed at the start terminus of the 17 mm beam and allowed to find and reach the dark box. Mice that did not find the dark goal box in 60 seconds were gently guided to the box. All mice were allowed to rest 10 seconds in the dark goal box before returned to home cage. On testing day, mice were individually placed at the start terminus of 17 mm and 11 mm beams sequentially. The time to cross the 80 cm in the center of the beam was recorded. Falls off the beam were recorded, and mice were given three total attempts to cross successfully. The maximum time per trial was 35s, which was assigned to mice that failed to cross during that period.

Gait Analysis.

Gait analysis was performed using the footprint test. Non-toxic water-based paint was used to mark each mouse forepaw (red) and hindpaw (black). Mice were placed in a brightly lit corridor runway, 60 cm in length to a dark goal box at the end of the corridor. Mice underwent two habituation trials, prior to assessment runs. Front base, hind base, forelimb stride, hindlimb stride, were assessed by measurement on corridor paper.

### Quantification and Statistical Analysis

Statistical Analysis.

In this study, the “n” represents number of animals used in experiments. For quantification in brain sections, we used at least three sections per animal. The number of brain sections used for quantification is presented in the figure legends. All bar graphs are mean values±SEM. Statistical comparisons between two groups were made using unpaired, two-tailed, Student's t test. In FIG. 60, a one sample t-test was used to compare mean values of the samples to the hypothetical value of 0. For group assessments, one-way ANOVA with post-hoc analysis was performed. All statistical analyses were performed in either R programming language or using GraphPad Prism.

DATA AND CODE AVAILABILITY. The bulk RNA-seq dataset (FIG. 4E, 11C) is available at the NCBI BioProject, accession no: PRJNA453419. The bulk RNA-seq datasets generated in (FIG. 11C) are available through GEO: GSE83376 and GEO: GSE75246, respectively. The microarray dataset (FIG. 11C) is available thorough GEO: GSE121409. The single-cell RNA-seq dataset generated by in (FIG. 7) is available through GEO: GSE98969

### REFERENCES

Ajami, B., Bennett, J. L., Krieger, C., Tetzlaff, W., and Rossi, F. M. V. (2007). Local self-renewal can sustain CNS microglia maintenance and function throughout adult life. Nat. Neurosci. 10, 1538-1543.
Ajami, B., Bennett, J. L., Krieger, C., McNagny, K. M., and Rossi, F. M. V. (2011). Infiltrating monocytes trigger EAE progression, but do not contribute to the resident microglia pool. Nat. Neurosci. 14, 1142-1149.
Bak, R. O., Dever, D. P., and Porteus, M. H. (2018). CRISPR/Cas9 genome editing in human hematopoietic stem cells. Nat. Protoc. 13, 358-376.
Bechmann, I., Galea, I., and Perry, V. H. (2007). What is the blood-brain barrier (not)? Trends Immunol. 28, 5-11.
Beck, M. (2007). New therapeutic options for lysosomal storage disorders: enzyme replacement, small molecules and gene therapy. Hum. Genet. 121, 1-22.
Beers, D. R., Henkel, J. S., Xiao, Q., Zhao, W., Wang, J., Yen, A. A., Siklos, L., McKercher, S. R.,
and Appel, S. H. (2006). Wild-type microglia extend survival in PU.1 knockout mice with familial amyotrophic lateral sclerosis. Proc. Natl. Acad. Sci. U.S.A 103, 16021-16026.
Bennett, F. C., Bennett, M. L., Yaqoob, F., Mulinyawe, S. B., Grant, G. A., Hayden Gephart, M., Plowey, E. D., and Barres, B. A. (2018). A Combination of Ontogeny and CNS Environment Establishes Microglial Identity. Neuron 98, 1170-1183.e8.
Bennett, M. L., Bennett, F. C., Liddelow, S. A., Ajami, B., Zamanian, J. L., Fernhoff, N. B., Mulinyawe, S. B., Bohlen, C. J., Adil, A., Tucker, A., et al. (2016). New tools for studying microglia in the mouse and human CNS. Proc. Natl. Acad. Sci. U.S.A 113, E1738-1746.
Biffi, A. (2017). Hematopoietic Stem Cell Gene Therapy for Storage Disease: Current and New Indications. Mol. Ther. J. Am. Soc. Gene Ther. 25, 1155-1162.
Bruttger, J., Karram, K., W6rtge, S., Regen, T., Marini, F., Hoppmann, N., Klein, M., Blank, T., Yona, S., Wolf, Y., et al. (2015). Genetic Cell Ablation Reveals Clusters of Local Self-Renewing Microglia in the Mammalian Central Nervous System. Immunity 43, 92-106.
Butovsky, O., Jedrychowski, M. P., Moore, C. S., Cialic, R., Lanser, A. J., Gabriely, G., Koeglsperger, T., Dake, B., Wu, P. M., Doykan, C. E., et al. (2014). Identification of a unique TGF-β-dependent molecular and functional signature in microglia. Nat. Neurosci. 17, 131-143.
Capotondo, A., Milazzo, R., Politi, L. S., Quattrini, A., Palini, A., Plati, T., Merella, S., Nonis, A., di Serio, C., Montini, E., et al. (2012). Brain conditioning is instrumental for successful microglia reconstitution following hematopoietic stem cell transplantation. Proc. Natl. Acad. Sci. U.S.A 109, 15018-15023.
Capotondo, A., Milazzo, R., Garcia-Manteiga, J. M., Cavalca, E., Montepeloso, A., Garrison, B. S., Peviani, M., Rossi, D. J., and Biffi, A. (2017). Intracerebroventricular delivery of hematopoietic progenitors results in rapid and robust engraftment of microglia-like cells. Sci. Adv. 3, el701211.
Cartier, N., Hacein-Bey-Abina, S., Bartholomae, C. C., Veres, G., Schmidt, M., Kutschera, I., Vidaud, M., Abel, U., Dal-Cortivo, L., Caccavelli, L., et al. (2009). Hematopoietic stem cell gene therapy with a lentiviral vector in X-linked adrenoleukodystrophy. Science 326, 818-823.
Chhabra, A., Ring, A. M., Weiskopf, K., Schnorr, P. J., Gordon, S., Le, A. C., Kwon, H.-S., Ring, N. G., Volkmer, J., Ho, P. Y., et al. (2016). Hematopoietic stem cell transplantation in immunocompetent hosts without radiation or chemotherapy. Sci. Transl. Med. 8, 351 ra105.
Cronk, J. C., Filiano, A. J., Louveau, A., Marin, I., Marsh, R., Ji, E., Goldman, D. H., Smirnov, I., Geraci, N., Acton, S., et al. (2018). Peripherally derived macrophages can engraft the brain independent of irradiation and maintain an identity distinct from microglia. J. Exp. Med. 215, 1627-1647.
Dagher, N. N., Najafi, A. R., Kayala, K. M. N., Elmore, M. R. P., White, T. E., Medeiros, R., West, B. L., and Green, K. N. (2015). Colony-stimulating factor 1 receptor inhibition prevents microglial plaque association and improves cognition in 3×Tg-A D mice. J. Neuroinflammation 12, 139.
Davalos, D., Grutzendler, J., Yang, G., Kim, J. V., Zuo, Y., Jung, S., Littman, D. R., Dustin, M. L., and Gan, W.-B. (2005). ATP mediates rapid microglial response to local brain injury in vivo. Nat. Neurosci. 8, 752-758.
Deczkowska, A., Keren-Shaul, H., Weiner, A., Colonna, M., Schwartz, M., and Amit, I. (2018). Disease-Associated Microglia: A Universal Immune Sensor of Neurodegeneration. Cell 173, 1073-1081.
Elmore, M. R. P., Najafi, A. R., Koike, M. A., Dagher, N. N., Spangenberg, E. E., Rice, R. A., Kitazawa, M., Matusow, B., Nguyen, H., West, B. L., et al. (2014). Colony-stimulating factor 1 receptor signaling is necessary for microglia viability, unmasking a microglia progenitor cell in the adult brain. Neuron 82, 380-397.
Erblich, B., Zhu, L., Etgen, A. M., Dobrenis, K., and Pollard, J. W. (2011). Absence of Colony Stimulation Factor-1 Receptor Results in Loss of Microglia, Disrupted Brain Development and Olfactory Deficits. PLoS ONE 6.
Füger, P., Hefendehl, J. K., Veeraraghavalu, K., Wendeln, A.-C., Schlosser, C., Obermüller, U., Wegenast-Braun, B. M., Neher, J. J., Martus, P., Kohsaka, S., et al. (2017). Microglia turnover with aging and in an Alzheimer's model via long-term in vivo single-cell imaging. Nat. Neurosci. 20, 1371-1376.
George, B. M., Kao, K. S., Kwon, H.-S., Velasco, B. J., Poyser, J., Chen, A., Le, A. C., Chhabra, A., Burnett, C. E., Cajuste, D., et al. (2019). Antibody Conditioning Enables MHC-Mismatched Hematopoietic Stem Cell Transplants and Organ Graft Tolerance. Cell Stem Cell 25, 185-192.e3. Ginhoux, F., Greter, M., Leboeuf, M., Nandi, S., See, P., Gokhan, S., Mehler, M. F., Conway, S. J., Ng, L. G., Stanley, E. R., et al. (2010). Fate mapping analysis reveals that adult microglia derive from primitive macrophages. Science 330, 841-845.
Goldmann, T., Wieghofer, P., Müller, P. F., Wolf, Y., Varol, D., Yona, S., Brendecke, S. M., Kierdorf, K., Staszewski, O., Datta, M., et al. (2013). A new type of microglia gene targeting shows TAK1 to be pivotal in CNS autoimmune inflammation. Nat. Neurosci. 16, 1618-1626.
Gomez Perdiguero, E., Klapproth, K., Schulz, C., Busch, K., Azzoni, E., Crozet, L., Garner, H., Trouillet, C., de Bruijn, M. F., Geissmann, F., et al. (2015). Tissue-resident macrophages originate from yolk-sac-derived erythro-myeloid progenitors. Nature 518, 547-551.
Gosselin, D., Link, V. M., Romanoski, C. E., Fonseca, G. J., Eichenfield, D. Z., Spann, N. J., Stender, J. D., Chun, H. B., Garner, H., Geissmann, F., et al. (2014). Environment drives selection and function of enhancers controlling tissue-specific macrophage identities. Cell 159, 1327-1340.
Guerreiro, R., Wojtas, A., Bras, J., Carrasquillo, M., Rogaeva, E., Majounie, E., Cruchaga, C., Sassi, C., Kauwe, J. S. K., Younkin, S., et al. (2013). TREM2 variants in Alzheimer's disease. N. Engl. J. Med. 368, 117-127.
Heneka, M. T., Carson, M. J., El Khoury, J., Landreth, G. E., Brosseron, F., Feinstein, D. L., Jacobs, A. H., Wyss-Coray, T., Vitorica, J., Ransohoff, R. M., et al. (2015). Neuroinflammation in Alzheimer's disease. Lancet Neurol. 14, 388-405.
Huang, Y., Xu, Z., Xiong, S., Sun, F., Qin, G., Hu, G., Wang, J., Zhao, L., Liang, Y.-X., Wu, T., et al. (2018). Repopulated microglia are solely derived from the proliferation of residual microglia after acute depletion. Nat. Neurosci. 21, 530-540.
Keren-Shaul, H., Spinrad, A., Weiner, A., Matcovitch-Natan, O., Dvir-Szternfeld, R., Ulland, T. K., David, E., Baruch, K., Lara-Astaiso, D., Toth, B., et al. (2017). A Unique Microglia Type Associated with Restricting Development of Alzheimer's Disease. Cell 169, 1276-1290.e17.
Kierdorf, K., Erny, D., Goldmann, T., Sander, V., Schulz, C., Perdiguero, E. G., Wieghofer, P., Heinrich, A., Riemke, P., H6lscher, C., et al. (2013a). Microglia emerge from erythromyeloid precursors via Pu.1- and Irf8-dependent pathways. Nat. Neurosci. 16, 273-280.
Kierdorf, K., Katzmarski, N., Haas, C. A., and Prinz, M. (2013b). Bone marrow cell recruitment to the brain in the absence of irradiation or parabiosis bias. PloS One 8, e58544.
Krasemann, S., Madore, C., Cialic, R., Baufeld, C., Calcagno, N., El Fatimy, R., Beckers, L., O'Loughlin, E., Xu, Y., Fanek, Z., et al. (2017). The TREM2-APOE Pathway Drives the Transcriptional Phenotype of Dysfunctional Microglia in Neurodegenerative Diseases. Immunity 47, 566-581.e9.
Lampron, A., Lessard, M., and Rivest, S. (2012). Effects of myeloablation, peripheral chimerism, and whole-body irradiation on the entry of bone marrow-derived cells into the brain. Cell Transplant. 21, 1149-1159.
Lavin, Y., Winter, D., Blecher-Gonen, R., David, E., Keren-Shaul, H., Merad, M., Jung, S., and Amit, I. (2014). Tissue-resident macrophage enhancer landscapes are shaped by the local microenvironment. Cell 159, 1312-1326.
Leyns, C. E. G., Ulrich, J. D., Finn, M. B., Stewart, F. R., Koscal, L. J., Serrano, J. R., Robinson, G. O., Anderson, E., Colonna, M., and Holtzman, D. M. (2017). TREM2 deficiency attenuates neuroinflammation and protects against neurodegeneration in a mouse model of tauopathy. Proc. Natl. Acad. Sci. 114, 11524-11529.
Li, Q., and Barres, B. A. (2018). Microglia and macrophages in brain homeostasis and disease. Nat. Rev. Immunol. 18, 225-242.
Lund, H., Pieber, M., Parsa, R., Han, J., Grommisch, D., Ewing, E., Kular, L., Needhamsen, M., Espinosa, A., Nilsson, E., et al. (2018). Competitive repopulation of an empty microglial niche yields functionally distinct subsets of microglia-like cells. Nat. Commun. 9, 1-13.
Mass, E., Jacome-Galarza, C. E., Blank, T., Lazarov, T., Durham, B. H., Ozkaya, N., Pastore, A., Schwabenland, M., Chung, Y. R., Rosenblum, M. K., et al. (2017). A somatic mutation in erythro-myeloid progenitors causes neurodegenerative disease. Nature 549, 389-393.
Mildner, A., Schmidt, H., Nitsche, M., Merkler, D., Hanisch, U.-K., Mack, M., Heikenwalder, M., Brück, W., Priller, J., and Prinz, M. (2007). Microglia in the adult brain arise from Ly-6ChiCCR2+ monocytes only under defined host conditions. Nat. Neurosci. 10, 1544-1553.
Mosher, K. I., and Wyss-Coray, T. (2014). Microglial dysfunction in brain aging and Alzheimer's disease. Biochem. Pharmacol. 88, 594-604.
Nimmerjahn, A., Kirchhoff, F., and Helmchen, F. (2005). Resting microglial cells are highly dynamic surveillants of brain parenchyma in vivo. Science 308, 1314-1318.
Olah, M., Patrick, E., Villani, A.-C., Xu, J., White, C. C., Ryan, K. J., Piehowski, P., Kapasi, A., Nejad, P., Cimpean, M., et al. (2018). A transcriptomic atlas of aged human microglia. Nat. Commun. 9, 539.
Prinz, M., and Priller, J. (2014). Microglia and brain macrophages in the molecular age: from origin to neuropsychiatric disease. Nat. Rev. Neurosci. 15, 300-312.
Prokop, S., Miller, K. R., Drost, N., Handrick, S., Mathur, V., Luo, J., Wegner, A., Wyss-Coray, T., and Heppner, F. L. (2015). Impact of peripheral myeloid cells on amyloid-β pathology in Alzheimer's disease—like mice. J. Exp. Med. 212, 1811-1818.
Rademakers, R., Baker, M., Nicholson, A. M., Rutherford, N. J., Finch, N., Soto-Ortolaza, A., Lash, J., Wider, C., Wojtas, A., DeJesus-Hernandez, M., et al. (2012). Mutations in the colony stimulating factor 1 receptor (CSF1R) gene cause hereditary diffuse leukoencephalopathy with spheroids. Nat. Genet. 44, 200-205.
Ransohoff, R. M., and Perry, V. H. (2009). Microglial physiology: unique stimuli, specialized responses. Annu. Rev. Immunol. 27, 119-145.
Salter, M. W., and Stevens, B. (2017). Microglia emerge as central players in brain disease. Nat. Med. 23, 1018-1027.
Seita, J., and Weissman, I. L. (2010). Hematopoietic stem cell: self-renewal versus differentiation. Wiley Interdiscip. Rev. Syst. Biol. Med. 2, 640-653.
Shechter, R., London, A., Varol, C., Raposo, C., Cusimano, M., Yovel, G., Rolls, A., Mack, M., Pluchino, S., Martino, G., et al. (2009). Infiltrating Blood-Derived Macrophages Are Vital Cells Playing an Anti-inflammatory Role in Recovery from Spinal Cord Injury in Mice. PLOS Med. 6, el 000113.
Shemer, A., Grozovski, J., Tay, T. L., Tao, J., Volaski, A., S013, P., Ardura-Fabregat, A., Gross-Vered, M., Kim, J.-S., David, E., et al. (2018). Engrafted parenchymal brain macrophages differ from microglia in transcriptome, chromatin landscape and response to challenge. Nat. Commun. 9, 1-16.
Shibuya, Y., Chang, C. C. Y., Huang, L.-H., Bryleva, E. Y., and Chang, T.-Y. (2014). Inhibiting ACAT1/SOAT1 in Microglia Stimulates Autophagy-Mediated Lysosomal Proteolysis and Increases Aβ1-42 Clearance. J. Neurosci. 34, 14484-14501.
Varvel, N. H., Grathwohl, S. A., Baumann, F., Liebig, C., Bosch, A., Brawek, B., Thal, D. R., Charo, I. F., Heppner, F. L., Aguzzi, A., et al. (2012). Microglial repopulation model reveals a robust homeostatic process for replacing CNS myeloid cells. Proc. Natl. Acad. Sci. U.S.A 109, 18150-18155.
Wang, Q., Tang, X. N., and Yenari, M. A. (2007). The inflammatory response in stroke. J. Neuroimmunol. 184, 53-68.
Wilkinson, A. C., Ishida, R., Kikuchi, M., Sudo, K., Morita, M., Crisostomo, R. V., Yamamoto, R., Loh, K. M., Nakamura, Y., Watanabe, M., et al. (2019). Long-term ex vivo haematopoietic-stem-cell expansion allows nonconditioned transplantation. Nature 571, 117-121.
Wilkinson, F. L., Sergijenko, A., Langford-Smith, K. J., Malinowska, M., Wynn, R. F., and Bigger, B. W. (2013). Busulfan conditioning enhances engraftment of hematopoietic donor-derived cells in the brain compared with irradiation. Mol. Ther. J. Am. Soc. Gene Ther. 21, 868-876.
Wyss-Coray, T., and Rogers, J. (2012). Inflammation in Alzheimer Disease-A Brief Review of the Basic Science and Clinical Literature. Cold Spring Harb. Perspect. Med. 2, a006346.
Yamamoto, R., Wilkinson, A. C., Ooehara, J., Lan, X., Lai, C.-Y., Nakauchi, Y., Pritchard, J. K., and Nakauchi, H. (2018). Large-Scale Clonal Analysis Resolves Aging of the Mouse Hematopoietic Stem Cell Compartment. Cell Stem Cell 22, 600-607.e4.
Youshani, A. S., Rowlston, S., O'Leary, C., Forte, G., Parker, H., Liao, A., Telfer, B., Williams, K., Kamaly-Asl, I. D., and Bigger, B. W. (2019). Non-myeloablative busulfan chimeric mouse models are less pro-inflammatory than head-shielded irradiation for studying immune cell interactions in brain tumours. J. Neuroinflammation 16, 25.
Zhan, L., Krabbe, G., Du, F., Jones, I., Reichert, M. C., Telpoukhovskaia, M., Kodama, L., Wang, C., Cho, S.-H., Sayed, F., et al. (2019). Proximal recolonization by self-renewing microglia re-establishes microglial homeostasis in the adult mouse brain. PLoS Biol. 17, e3000134.

Each publication cited in this specification is hereby incorporated by reference in its entirety for all purposes.

It is to be understood that this invention is not limited to the particular methodology, protocols, cell lines, animal species or genera, and reagents described, as such may vary. It is also to be understood that the terminology used herein is for the purpose of describing particular embodiments only, and is not intended to limit the scope of the present invention, which will be limited only by the appended claims

As used herein the singular forms “a”, “and”, and “the” include plural referents unless the context clearly dictates otherwise. Thus, for example, reference to “a cell” includes a plurality of such cells and reference to “the culture” includes reference to one or more cultures and equivalents thereof known to those skilled in the art, and so forth. All technical and scientific terms used herein have the same meaning as commonly understood to one of ordinary skill in the art to which this invention belongs unless clearly indicated otherwise.

1. A method for the efficient replacement of endogenous microglial cells in an individual with donor circulation-derived myeloid cells (CDMC), the method comprising:
performing hematopoietic stem cell transplantation (HSCT) by (i) ablation of endogenous hematopoietic stem cells; and (ii) infusion of donor hematopoietic stem cells to the individual; and
(iii) administering a microglial cell conditioning factor;
wherein a stable, high level of chimerism in the brain-resident microglial cell population is achieved.

2. The method of claim 1, wherein the microglial cell conditioning agent is an inhibitor of colony stimulating factor 1 (CSF-1) signaling pathway.

3. The method of claim 1, wherein the microglial cell conditioning agent is a brain-penetrant inhibitor of CSF1R.

4. The method of claim 1, wherein the microglial cell conditioning agent is initiated from about 14 to about 28 days following infusion of the HSC.

5. The method of claim 1, wherein the microglial cell conditioning agent is administered for a period of from 4 to 10 days.

6. The method of claim 1, wherein following administration of the microglial cell conditioning agent, the individual is at least 50% chimeric for host-derived microglial cells.

7. The method of claim 1, wherein the individual maintains high levels of chimerism for at least about 12 weeks.

8. The method of claim 1, wherein the HSCT comprises administration of purified hematopoietic stem cells.

9. The method of claim 1, wherein donor HSC are genetically modified to express a gene of interest.

10. The method of claim 1, wherein (i) ablation of endogenous HSC is performed with myeloablative conditioning.

11. The method of claim 10, wherein myeloablative conditioning comprises administration of busulfan.

12. The method of claim 1, wherein (i) ablation of endogenous HSC is accomplished by administration of a chemotherapeutic agent or combination of agents.

13. The method of claim 12, wherein the chemotherapeutic agent is a brain-penetrant agent.

14. The method of claim 1, wherein (i) ablation of endogenous HSC is performed with non-myeloablative conditioning in the absence of busulfan and combined with guided ultrasound.

15. The method of claim 9, wherein the gene of interest is therapeutic for treatment of a neurologic condition.

16. The method of claim 15, wherein the neurologic condition is selected from Alzheimer's disease; brain cancer; and a lysosomal storage disease including Gaucher disease.

17-19. (canceled)

20. A method for the efficient replacement of endogenous microglial cells in an individual with a lysosomal storage disease with circulation-derived myeloid cells (CDMC) that correct the enzymatic defect of the lysosomal storage disease, the method comprising:
performing hematopoietic stem cell transplantation (HSCT) by (i) ablation of endogenous hematopoietic stem cells; and (ii) infusion of donor hematopoietic stem cells to the individual that correct the enzymatic defect of the lysosomal storage disease; and
(iii) administering a microglial cell conditioning factor;
wherein a stable, high level of chimerism in the brain-resident microglial cell population that correct the enzymatic defect of the lysosomal storage disease is achieved.

21. The method of claim 21, wherein the lysosomal storage disease is Gaucher disease.

22. The method of claim 21, wherein the donor hematopoietic stem cells express functional galacto-cerebrosidase (GCase), encoded by the GBA1 gene, wherein the donor HSC are genetically corrected at the GBA1 locus or the donor HSC comprise a normal endogenous gene at the GBA1 locus.

23-24. (canceled)


SEQUENCE LISTING (20 sequences)

SEQ ID NO: 1 - DNA, 20 bp, Artificial sequence
  synthetic sequence
aagactcacgtggacctgct

SEQ ID NO: 2 - DNA, 20 bp, Artificial sequence
  synthetic sequence
cggttattcaacttgcacca

SEQ ID NO: 3 - DNA, 21 bp, Artificial sequence
  synthetic sequence
aggatgttgacttccgagttg

SEQ ID NO: 4 - DNA, 20 bp, Artificial sequence
  synthetic sequence
aagggagctgcagtggagta

SEQ ID NO: 5 - DNA, 20 bp, Artificial sequence
  synthetic sequence
ccgaaaatctgtgggaagtc

SEQ ID NO: 6 - DNA, 19 bp, Artificial sequence
  synthetic sequence
ctgttcctgtacggcatgg

SEQ ID NO: 7 - DNA, 20 bp, Artificial sequence
  synthetic sequence
ggcattaaagcagcgtatcc

SEQ ID NO: 8 - DNA, 22 bp, Artificial sequence
  synthetic sequence
ttcagcaagttcccagcttcgg

SEQ ID NO: 9 - DNA, 21 bp, Artificial sequence
  synthetic sequence
gagcccaattttagcaagaga

SEQ ID NO: 10 - DNA, 20 bp, Artificial sequence
  synthetic sequence
ggatggggaacgcagctctt

SEQ ID NO: 11 - DNA, 19 bp, Artificial sequence
  synthetic sequence
agtgcggcagaatacagca

SEQ ID NO: 12 - DNA, 27 bp, Artificial sequence
  synthetic sequence
  misc_feature (1)..(1): The nucleotide at position 1 comprises hexachlorofluorescein
  misc_feature (9)..(10): The nucleotides at positions 9 and 10 comprise a ZEN quencher
  misc_feature (27)..(27): The nucleotide at position 27 comprises a 3IABkFQ quencher
tgatgggttgtgaaggcagctgcacct

SEQ ID NO: 13 - DNA, 22 bp, Artificial sequence
  synthetic sequence
catatactgtaaagactaggag

SEQ ID NO: 14 - DNA, 21 bp, Artificial sequence
  synthetic sequence
  misc_feature (1)..(1): The nucleotide at position 1 comprises a FAM fluorescin derivitive
  misc_feature (9)..(10): The nucleotides at positions 9 and 10 comprise a ZEN quencher
  misc_feature (21)..(21): The nucleotide at position 21 comprises a 3IABkFQ quencher
tggacaccccaccacagccaa

SEQ ID NO: 15 - DNA, 24 bp, Artificial sequence
  synthetic sequence
ctgttaacataaatattggtccag

SEQ ID NO: 16 - DNA, 21 bp, Artificial sequence
  synthetic sequence
cacagaagtcattcagaacac

SEQ ID NO: 17 - DNA, 28 bp, Artificial sequence
  synthetic sequence
  misc_feature (1)..(1): The nucleotide at position 1 comprises a FAM fluorescin derivitive
  misc_feature (9)..(10): The nucleotides at positions 9 and 10 comprise a ZEN quencher
  misc_feature (28)..(28): The nucleotide at position 28 comprises a 3IABkFQ quencher
cttcaggtacaacaaaccattcacaatc

SEQ ID NO: 18 - DNA, 21 bp, Artificial sequence
  synthetic sequence
cctacacagagagaaataccc

SEQ ID NO: 19 - DNA, 21 bp, Artificial sequence
  synthetic sequence
caactgcaggctgtaaaatgc

SEQ ID NO: 20 - DNA, 27 bp, Artificial sequence
  synthetic sequence
  misc_feature (1)..(1): The nucleotide at position 1 comprises a FAM fluorescin derivative
  misc_feature (9)..(10): The nucleotides at positions 9 and 10 comprise a ZEN quencher
  misc_feature (27)..(27): The nucleotide at position 27 comprises a 3IABkFQ quencher
tcagcctcatcggagggctaaagtgtc